(19) United States
(12) Patent Application Publication
(10) Pub. No.: US 2021/0254182 A1
(43) Pub. Date: Aug. 19, 2021

(54) METHODS AND TOOLS FOR PLANT PATHOGEN ASSESSMENT

(71) Applicant: UNIVERSITÉ LAVAL, Québec (CA)

(72) Inventors: Geneviève ARSENAULT-LABRECQUE, Québec (CA); Chloé DUSSAULT-BENOIT, Lévis (CA); Richard R. BELANGER, Québec (CA); Humira SONAH, Srinagar (IN); François BELZILE, Québec (CA)

(21) Appl. No.: 16/973,317

(22) PCT Filed: Jun. 18, 2019
(86) PCT No.: PCT/CA2019/050856

§ 371 Date: Dec. 8, 2020

Related U.S. Application Data
(60) Provisional application No. 62/686,242, filed on Jun. 18, 2018.

(51) Int. Cl. C12Q 1/6895; A01H 1/04

(52) U.S. Cl. CPC C12Q 1/6895; C12Q 2600/156; A01H 1/04

(57) ABSTRACT

Described herein are methods, products and tools for plant pathogen assessment and management. Also described are collections, kits and packages comprising reagents (e.g. oligonucleotides) and uses thereof, for example for plant pathogen assessment. In an embodiment, the pathogen is a Phytophthora pathogen, in a further embodiment Phytophthora sojae. In an embodiment the plant is soybean.

### CROSS-REFERENCE TO RELATED APPLICATIONS

The present application claims the benefit of U.S. Provisional Application Ser. No. 62/686,242 filed on Jun. 18, 2018, which is incorporated herein by reference in its entirety.

### SEQUENCE LISTING

This application contains a Sequence Listing in computer readable form entitled “G11229_399_SeqList.txt”, created on Jun. 18, 2019 and having a size of about 33,873 KB. The computer readable form is incorporated herein by reference in its entirety.

### TECHNICAL FIELD

The present invention generally relates to plant pathogen assessment, and more particularly to the assessment of Phytophthora pathogens.

### BACKGROUND ART

Phytophthora sojae (Kauf. & Gerd.), a hemibiotrophic oomycete causing stem and root rot in soybean, is among the top ten plant-pathogenic oomycetes/fungi of both scientific and economic importance (Kamoun et al. 2015). Management of P. sojae relies mostly on the development of cultivars with major resistance (Rps) genes. The development of stem and root rot caused by P. sojae is determined by the gene-for-gene relationship between resistance (Rps) genes in soybean and their matching avirulence (Avr) genes in the pathogen. Typically, Rps genes code for proteins having nucleotide-binding site (NBS) and leucine-rich repeat (LRR), while P. sojae Avr genes code for small effector proteins mostly with RXLR and DEER amino acid motifs. The NBS-LRR proteins from soybean recognize the RXLR effectors encoded by Avr genes from P. sojae, inducing an appropriate defense response (Sahoo et al. 2017; Song et al. 2013). The pathogen can avoid recognition conferred by Rps genes through various mutations such as a substitutions, frameshift mutations, partial or complete deletions, large insertions, recombinations, or changes in expression of Avr genes (Tyler and Gijzen 2014; Goss et al. 2013).

To date, over 27 major Rps genes have been identified in soybean (Sahoo et al. 2017) and about 12 Avr genes have been identified and characterized in P. sojae (Gijzen et al. 1996; May et al. 2002; MacGregor et al. 2002; Whisson 1995; Tyler et al. 1995). Most of the Avr genes are clustered together on P. sojae chromosomes, and many of them are candidate paralogs. For instance, Avr1a and Avr1c have very similar sequences (Na et al. 2014). In addition, some of the gene pairs earlier thought to be different genes, such as Avr3a/Avr5 and Avr6/Avr4, turned out to be different alleles of the same gene (Dong et al. 2011; Dou et al. 2010). In the case of Avr1a, deletion of two out of four nearly identical copies of the gene have been found to cause virulence. Similarly, some P. sojae strains have as many as four paralogs of Avr3a, and some have only one (Qutob et al. 2009). Such high levels of similarity, tandem duplications and variation in the number of copies make it very difficult to develop sequence-based diagnostic markers.

Avirulence (Avr) genes are mostly located in highly dynamic genome areas containing duplications and repetitive sequences that are prone to chromosomal rearrangements. High levels of sequence variation, duplications, interdependency of Avr genes and rapid evolution complicate the task of characterizing newly evolved strains. Efficient tools to rapidly and accurately identify virulence features in P. sojae have become essential to prevent disease outbreaks. The objective is to identify variation signatures (haplotypes) associated with virulence factors. Haplotypes representing the allelic variation of a given gene have also been found to be tightly linked with the copy number variation and expression of the same gene (Kadam et al. 2016; Verta, Landry, and MacKay 2016; Zeng, Zhou, and Huang 2017).

Precise phenotyping of the interactions between pathotypes and differentials remains an essential component to assess the functionality of either Avror Rps genes. For this purpose, several phenotyping methods have been developed and proposed (Haas and Buzzell 1976; Kilen, Hartwig, and Keeling 1974; Ward et al. 1979; Morrison and Thorne 1978; Wagner and Wilkinson 1992; Pazdernik et al. 2007). Over the years, the hypocotyl inoculation test has become the standard test, particularly because of its ease of use (Dorrance, Jia, and Abney 2004). However, as convenient as the hypocotyl inoculation method is, it has limitations leading to the identification of false positives or negatives (Schmitthenner, Hobe, and Bhat 1994), which can bring confusion about the presence and/or functionality of Avr genes in P. sojae isolates. Recently, Lebreton et al. (2018) proposed the use of a simplified hydroponic assay as a way to more robustly characterize the phenotypes by inoculating the root system of soybean plants directly with zoospores of P. sojae.

There is therefore a need for further development of methods and tools for plant pathogen assessment.

The present description refers to a number of documents, the content of which is herein incorporated by reference in their entirety.

### SUMMARY OF THE INVENTION

The present invention relates to plant pathogen assessment and management, such as the assessment and management of Phytophthora pathogens.

In various aspects and embodiments, the present disclosure provides the following items:
1. A method for assessing whether a Phytophthora pathogen is virulent or avirulent, comprising:

(a) determining, in a sample comprising Phytophthora nucleic acid, the presence or absence of one or more variations in one or more Avr genes or a flanking region thereof in the Phytophthora nucleic acid; and

(b) determining whether the Phytophthora pathogen is virulent or avirulent on the basis of the presence or absence of the one or more variations.
2. The method of item 1, wherein the one or more Avr genes are one or more of Avr1a, Avr1b, Avr1c, Avr1d, Avr1k, Avr3a and Avr6.
3. The method of item 1 or 2, wherein the one or more variations are each independently a substitution, deletion or insertion of one or more nucleotides.
4. The method of item 3, wherein the one or more variations are a single nucleotide polymorphism (SNP).
5. The method of any one of items 1 to 4, wherein the variations are one or more indels and/or SNPs corresponding to one or more indels and/or SNPs set forth in FIGS. 1 (Avr1a), 3 (Avr1b), 5 (Avr1c), 7 (Avr1d), 9 (Avr1k), 11 (Avr3a) and/or 12 (Avr6), 16-23 and/or Table 4 and/or 5.
6. The method of any one of items 1 to 5, wherein the presence or absence of the one or more variations is determined via assessment of a different region of the Phytophthora nucleic acid that is in linkage disequilibrium with the one or more variations.
7. The method of any one of items 1 to 6, wherein the presence or absence of the one or more variations is determined using an amplification method.
8. The method of item 7, wherein the amplification method is polymerase chain reaction (PCR).
9. The method of item 7 or 8, wherein the amplification reaction is carried out using primer sequences comprised within the sequences set forth in one or more of FIGS. 16-22.
10. The method of any one of items 7 to 9, wherein the amplification is carried out as one or more multiplex amplifications for determination of the presence or absence of two or more variations in each amplification reaction.
11. The method of item 10, wherein the one or more multiplex amplifications comprises or consists of two multiplex amplifications.
12. The method of item 11, wherein the two multiplex amplifications comprise (a) a first multiplex amplification to determine the presence or absence of one or more indels and/or SNPs corresponding to one or more indels and/or SNPs in Avr1a shown in FIGS. 1, 16 and/or 23, and/or Table 4 and/or 5 one or more indels and/or SNPs corresponding to one or more indels and/or SNPs in Avr1b, Avr1d, Avr1k, Avr3a and Avr6 shown in FIGS. 3, 7, 9, 11, 13 and/or 17 and 19-23, and/or Table 4 and/or 5; and (b) a second amplification to determine the presence or absence of one or more indels and/or SNPs corresponding to one or more indels and/or SNPs in Avr1c shown in FIGS. 5, 18, and/or 23, and/or Table 4 and/or 5.
13. The method of any one of items 7 to 12, wherein the amplification is performed using one or more primer pairs set forth in Table 2, or functional equivalents thereof that are targeted to a sequence up to 200 nucleotides upstream or downstream from the regions targeted by the one or more primers set forth in Table 2.
14. The method of item 13, wherein the amplification is performed using one or more primer pairs targeted to a sequence up to 150 nucleotides upstream or downstream from the regions targeted by the one or more primers set forth in Table 2.
15. The method of item 13 or 14, wherein the amplification is performed using one or more primer pairs targeted to a sequence up to 100 nucleotides upstream or downstream from the regions targeted by the one or more primers set forth in Table 2.
16. The method of any one of items 13 to 15, wherein the amplification is performed using one or more primer pairs targeted to a sequence up to 50 nucleotides upstream or downstream from the regions targeted by the one or more primers set forth in Table 2.
17. The method of any one of items 1 to 16, wherein the Phytophthora pathogen is Phytophthora sojae.
18. A method for assessing risk of Phytophthora pathogen infection of a soybean plant:

(a) assessing, in a sample obtained from the plant, the soil, the water, the seeds, the air, or any culture containing one or several isolates of Phytophthora pathogen, whether the sample comprises a virulent or avirulent Phytophthora pathogen using the method of any one of items 1 to 17; and

(b) assessing the risk of Phytophthora pathogen infection of the soybean plant on the basis of the assessment made in (a), wherein the presence of a virulent Phytophthora pathogen in the sample is indicative of an elevated risk of Phytophthora pathogen infection of the soybean plant.
19. The method of item 19, further comprising treating the plant with an antifungal agent if the risk of Phytophthora pathogen infection is elevated.
20. A method for selecting a soybean cultivar for planting in an agricultural area, comprising:

(a) assessing, in a sample obtained from the plant, the soil, the water, the seeds, the air, or any culture containing one or several isolates of Phytophthora pathogen, whether the sample comprises a virulent or avirulent Phytophthora pathogen using the method of any one of items 1 to 17;

(b) if the sample comprises a virulent Phytophthora pathogen, selecting a soybean cultivar comprising one or more resistances (Rps) genes that confer resistance to the one or more Avr genes identified in the sample that confer virulence, for planting in the agricultural area.
21. A collection, kit or package comprising one or more oligonucleotides for determining the presence or absence of one or more variations in one or more Avrgenes or a flanking region thereof in the nucleic acid of a Phytophthora pathogen.
22. The collection, kit or package of item 21, wherein the one or more Avr genes are one or more of Avr1a, Avr1b, Avr1c, Avr1d, Avr1k, Avr3a and Avr6.
23. The collection, kit or package of item 21 or 22, wherein the one or more variations are each independently a substitution, deletion or insertion of one or more nucleotides.
24. The collection, kit or package of item 23, wherein the one or more variations are a single nucleotide polymorphism (SNP).
25. The collection, kit or package of any one of items 21 to 24, wherein the variations are one or more indels and/or SNPs corresponding to one or more indels and/or SNPs set forth in FIGS. 1 (Avr1a), 3 (Avr1b), 5 (Avr1c), 7 (Avr1d), 9 (Avr1k), 11 (Avr3a) and/or 12 (Avr6), 16-23 and/or Table 4 and/or 5
26. The collection, kit or package of any one of items 21 to 25, wherein the presence or absence of the one or more variations is determined via assessment of a different region of the Phytophthora nucleic acid that is in linkage disequilibrium with the one or more variations.
27. The collection, kit or package of any one of items 21 to 26, wherein the presence or absence of the one or more variations is determined using an amplification method.
28. The collection, kit or package of item 27, wherein the amplification method is polymerase chain reaction (PCR).
29. The collection, kit or package of item 27 or 28, wherein the one ore more oligonucleotides comprise primer sequences comprised within the sequences set forth in one or more of FIGS. 16-22 for use in one or more amplification reactions.
30. The collection, kit or package of any one of items 27 to 29, wherein the one or more oligonucleotides are for use in one or more multiplex amplifications for determination of the presence or absence of two or more variations in each amplification reaction.
31. The collection, kit or package of item 30, wherein the one or more multiplex amplifications comprises or consists of two multiplex amplifications.
32. The collection, kit or package of item 31, wherein the two multiplex amplifications comprise (a) a first multiplex amplification to determine the presence or absence of one or more indels and/or SNPs corresponding to one or more indels and/or SNPs in Avr1a shown in FIGS. 1, 16 and/or 23, and/or Table 4 and/or one or more indels and/or SNPs corresponding to one or more indels and/or SNPs in Avr1b, Avr1d, Avr1k, Avr3a and Avr6 shown in FIGS. 3, 7, 9, 11, 13 and/or 17 and 19-23, and/or Table 4 and/or 5; and (b) a second amplification to determine the presence or absence of one or more indels and/or SNPs corresponding to one or more indels and/or SNPs in Avr1c shown in FIGS. 5, 18, and/or 23, and/or Table 4 and/or 5.
33. The collection, kit or package of any one of items 27 to 32, wherein the one or more oligonucleotides comprise one or more primer pairs set forth in Table 2, or functional equivalents thereof that are targeted to a sequence up to 200 nucleotides upstream or downstream from the regions targeted by the one or more primers set forth in Table 2.
34. The collection, kit or package of item 33, wherein the one or more oligonucleotides comprise one or more primer pairs targeted to a sequence up to 150 nucleotides upstream or downstream from the regions targeted by the one or more primers set forth in Table 2.
35. The collection, kit or package of item 33 or 34, wherein the one or more oligonucleotides comprise one or primer pairs targeted to a sequence up to 100 nucleotides upstream or downstream from the regions targeted by the one or more primers set forth in Table 2.
36. The collection, kit or package of any one of items 33 to 35, wherein the one or more oligonucleotides comprise one or more primer pairs targeted to a sequence up to 50 nucleotides upstream or downstream from the regions targeted by the one or more primers set forth in Table 2.
37. The collection, kit or package of any one of items 21 to 36, wherein the Phytophthora pathogen is Phytophthora sojae.
38. The collection, kit or package of any one of items 21 to 36, wherein the one or more oligonucleotides are attached or bound to a solid support.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1: Structural and nucleotide diversity at the Avr1a locus among 31 isolates of Phytophthora sojae reveal distinct haplotypes associated with virulence phenotypes. a Variants in the vicinity of the P. sojae Avr1a gene. The yellow box represents the coding region of the gene. The orange box shows the location of the deletion. Asterisks (*) indicate approximate positions of the SNPs. Those SNPs are representative of a cluster of SNPs defining a haplotype. b Schematic graph of the position of the SNPs for each isolate, grouped by haplotypes. SNPs in gray background are different from the reference genome (isolate P6497). c Phenotypic response of the outliers (when the phenotype did not match the genotype based on the hypocotyl test) from the hydroponic assay. Responses showed here are representative of all isolates tested. *CNV of Avr1a gene for the reference genome (P6497) is based on data from Qutob et al. (Qutob et al. 2009).

FIG. 2: Agarose gel electrophoresis of the PCR reaction for Avr1a gene. The phenotype of avirulence (A) or virulence (V) on Rps1a for each selected isolate is indicated at the top of the gel. Primers were designed to get an amplification only when isolates are avirulent on Rps1a.

FIG. 3: Nucleotide diversity at the Avr1b locus among 31 isolates of Phytophthora sojae reveal distinct haplotypes associated with virulence phenotypes. a Variants within the coding region of the P. sojae Avr1b gene. Yellow box represents the coding region of the gene and gray bars, 5′ and 3′ UTR. Asterisks (*) indicate approximate positions of the SNPs and small indels. Those variants are representative of a cluster of variants defining a haplotype. b Schematic graph of the position of the SNPs for each isolate, grouped by haplotypes. Variants in gray background are different from the reference genome (isolate P6497). c Phenotypic response of the outliers (when the phenotype did not match the genotype based on the hypocotyl test) from the hydroponic assay. Responses showed here are representative of all isolates tested.

FIG. 4: Agarose gel electrophoresis of the PCR reaction for Avr1b gene. The phenotype of avirulence (A) or virulence (V) on Rps1b for each selected isolate is indicated at the top of the gel. Primers were designed to get an amplification only when isolates are avirulent on Rps1b.

FIG. 5: Structural and nucleotide diversity at the Avr1c locus among 31 isolates of Phytophthora sojae reveal distinct haplotypes associated with virulence phenotypes. a Variants within the coding region of the P. sojae Avr1c gene. Yellow box represents the coding region of the gene and gray bars, 5′ and 3′ UTR. Asterisks (*) indicate approximate positions of the SNPs. Those SNPs are representative of a cluster of SNPs defining a haplotype. b Schematic graph of the position of the SNPs for each isolate, grouped by haplotypes. SNPs in gray background are different from the reference genome (isolate P6497). c Phenotypic response of the outliers (when the phenotype did not match the genotype based on the hypocotyl test) from the hydroponic assay. Responses showed here are representative of all isolates tested.

FIG. 6: Agarose gel electrophoresis of the PCR reaction for Avr1c gene. The phenotype of avirulence (A) or virulence (V) on Rps1c for each selected isolate is indicated at the top of the gel. Primers were designed to get an amplification only when isolates are avirulent on Rps1c.

FIG. 7: Structural and nucleotide diversity at the Avr1d locus among 31 isolates of Phytophthora sojae reveal distinct haplotypes associated with virulence phenotypes. a Deletion in the vicinity of the P. sojae Avr1d locus. Yellow box represents exon and gray bars, 5′ and 3′ UTR. Orange boxes show the position of deletions in virulent isolates. b Schematic graph of the genotypes based on the deletion. Genotypes in gray background are different from the reference genome (isolate P6497). c Phenotypic response of the outliers (when the phenotype did not match the genotype based on the hypocotyl test) from the hydroponic assay. Responses showed here are representative of all isolates tested.

FIG. 8: Agarose gel electrophoresis of the PCR reaction for Avr1d gene. The phenotype of avirulence (A) or virulence (V) on Rps1d for each selected isolate is indicated at the top of the gel. Primers were designed to get an amplification only when isolates are avirulent on Rps1d.

FIG. 9: Nucleotide diversity at the Avr1k locus among 31 isolates of Phytophthora sojae reveal distinct haplotypes associated with virulence phenotypes. a Variants within the coding region of the Phytophthora sojae Avr1k gene. Yellow box represents the coding region of the gene and gray bars, 5′ and 3′ UTR. Asterisks (*) indicate approximate positions of the SNPs and small indel. Those variants are representative of a cluster of variants defining a haplotype. b Schematic graph of the position of the variants for each isolate, regrouped by haplotypes. Variants in gray background are different from the reference genome (isolate P6497). c Phenotypic response of the outliers (when the phenotype did not match the genotype based on the hypocotyl test) from the hydroponic assay. Responses showed here are representative of all isolates tested.

FIG. 10: Agarose gel electrophoresis of the PCR reaction for Avr1k gene. The phenotype of avirulence (A) or virulence (V) on Rps1k for each selected isolate is indicated at the top of the gel. Primers were designed to get an amplification only when isolates are avirulent on Rps1k.

FIG. 11: Structural and nucleotide diversity at the Avr3a locus among 31 isolates of Phytophthora sojae reveal distinct haplotypes associated with virulence phenotypes. a Variants in the coding region of the P. sojae Avr3a region. Yellow box represents the coding region of the gene and gray bars, 5′ and 3′ UTR. Asterisk (*) indicate approximate positions of the SNPs and small indel. Those variants are representative of a cluster of variants defining a haplotype. b Schematic graph of the position of the variants for each isolate, regrouped by haplotypes. Variants in gray background are different from the reference genome (isolate P6497). Phenotype results were confirmed by re-testing a number of isolates with the hydroponic assay)*CNV of Avr3a gene for the reference genome (P6497) is based on data from Qutob et al. (2009).

FIG. 12: Agarose gel electrophoresis of the PCR reaction for Avr3a gene. The phenotype of avirulence (A) or virulence (V) on Rps3a for each selected isolate is indicated at the top of the gel. Primers were designed to get an amplification only when isolates are avirulent on Rps3a.

FIG. 13: Structural and nucleotide diversity at the Avr6 locus among 31 isolates of Phytophthora sojae reveal distinct haplotypes associated with virulence phenotypes. a Variants in the upstream region of the P. sojae Avr6 gene. Yellow box represents exon and gray bars, 5′ and 3′ UTR. Asterisks (*) indicate approximate positions of the SNPs and small indel. b Schematic graph of the position of the variants for each isolate, regrouped by haplotypes. Variants in gray background are different from the reference genome (isolate P6497). c Phenotypic response of the outliers (when the phenotype did not match the genotype based on the hypocotyl test) from the hydroponic assay. Responses showed here are representative of all isolates tested.

FIG. 14: Agarose gel electrophoresis of the PCR reaction for Avr6 gene. The phenotype of avirulence (A) or virulence (V) on Rps6 for each selected isolate is indicated at the top of the gel. Primers were designed to get an amplification only when isolates are avirulent on Rps6.

FIG. 15: Gel images of multiplex PCR amplifications of discriminant regions associated with avirulence alleles for seven Avr genes in Phytophthora sojae. (A-B) Results obtained with 31 isolates with a known pathotype, as indicated at the bottom of the gel, for Avr1a, 1b, 1d, 1k, 3a and 6. Expected size of the amplicon for each Avr gene is indicated on the right. (C) Complementary gel of PCR amplification of discriminant region associated with the avirulence allele for Avr1c (right) along with results obtained for the 31 isolates (A=avirulent and V=virulent) where A or V indicates presence or absence of the amplicon, respectively. For each isolate, the pathotype should correspond to the absence of an amplicon for each corresponding gene.

FIG. 16: Alignment of sequences covering P. sojae Avr1a gene and associated regions, including SNPs and/or indels associated with Avr1a described herein (16A-16E: refgenome, consensus: SEQ ID NO: 1, haplotypes A-E: SEQ ID NOs: 2-6; 16F-16J: refgenome: SEQ ID NO: 7, haplotypes A-E: SEQ ID NOs: 8-11, consensus SEQ ID NO: 12; 16K-16N: refgenome: SEQ ID NO: 13, haplotypes A-E: SEQ ID NOs: 14-18, consensus SEQ ID NO: 19; 16O-16S: refgenome, consensus: SEQ ID NO: 20, haplotypes A-E: SEQ ID NOs: 21-25; 16T-16X: refgenome, consensus: SEQ ID NO: 26, haplotypes A-E: SEQ ID NOs: 27-31; 16Y-16CC: refgenome: SEQ ID NO: 32, haplotypes A-E: SEQ ID NOs: 33-37, consensus SEQ ID NO: 38; 16DD-16HH: refgenome, consensus: SEQ ID NO: 39, haplotypes A-E: SEQ ID NOs: 40-44).

FIG. 17: Alignment of sequences covering P. sojae Avr1b gene and associated regions, including SNPs and/or indels associated with Avr1b described herein (refgenome: SEQ ID NO: 45, haplotypes A-C: SEQ ID NOs: 46-48, consensus SEQ ID NO: 49).

FIG. 18: Alignment of sequences covering P. sojae Avr1c gene and associated regions, including SNPs and/or indels associated with Avr1c described herein (refgenome: SEQ ID NO: 50, haplotypes A-E: SEQ ID NOs: 51-55, consensus SEQ ID NO: 56).

FIG. 19: Alignment of sequences covering P. sojae Avr1d gene and associated regions, including SNPs and/or indels associated with Avr1d described herein (refgenome, consensus: SEQ ID NO: 57, haplotypes A-C: SEQ ID NOs: 58-60).

FIG. 20: Alignment of sequences covering P. sojae Avr1k gene and associated regions, including SNPs and/or indels associated with Avr1k described herein (refgenome: SEQ ID NO: 61, haplotypes A-C: SEQ ID NOs: 62-64).

FIG. 21: Alignment of sequences covering P. sojae Avr3a gene and associated regions, including SNPs and/or indels associated with Avr3a described herein (refgenome, consensus: SEQ ID NO: 65, haplotypes A-B: SEQ ID NOs: 66-67).

FIG. 22: Alignment of sequences covering P. sojae Avr6 gene and associated regions, including SNPs and/or indels associated with Avr6 described herein (refgenome, consensus: SEQ ID NO: 68, haplotypes A-B: SEQ ID NOs: 69-70).

FIG. 23: Discriminant haplotypes associated with distinct phenotypes in seven avirulence genes of Phytophthora sojae used to build discriminant primers. (A) Avr1a, (B) Avr1b, (C) Avr1c, (D) Avr1d, (E) Avr1k, (F) Avr3a, and (G) Avr6. A=avirulent and V=virulent.

FIG. 24: Comparison of molecular and phenotyping assays to determine the pathotypes of Phytophthora sojae isolates. (A) Gel image of multiplex PCR amplifications of discriminant regions associated with avirulence alleles for seven Avr genes in P. sojae isolate 2012-82. Presence of amplicons for Avr1b, 1d and 1k predicts a pathotype 1a, 1c, 3a and 6. (B) Phenotyping results for isolate 2012-82 indicates a compatible interaction with Harosoy (no Rps), Rps1a, Rps1c, Rps3a and Rps6 and an incompatible interaction with Rps1b, Rps1d and Rps1k thereby assessing a pathotype 1a, 1c, 3a and 6, similar to the molecular assay. (C) Gel image of multiplex PCR amplifications of discriminant regions associated with avirulence alleles for seven Avr genes in P. sojae isolate 2012-156. Presence of amplicons for Avr1b, 1k, 3a, 6 and all three amplicons for Avr1a predicts a pathotype 1c and 1d. (D) Phenotyping results for isolate 2012-156 indicates a compatible interaction with Harosoy (no Rps), Rps1c, Rps1d and Rps3a and an incompatible interaction with Rps1a, Rps1b, Rps1k and Rps6 thereby assessing a pathotype 1c, 1d, and 3a, with 3a being the only interaction at odds with the molecular assay.

### DISCLOSURE OF INVENTION

In the studies described herein, a diverse set of 31 P. sojae isolates representing the range of pathotypes commonly observed in soybean fields were sequenced using whole genome sequencing (WGS). To understand the evolution and genetic constitution of P. sojae strains, haplotype analyses using the WGS data were performed for the seven most important Avr genes found in P. sojae populations: 1a, 1b, 1c, 1d, 1k, 3a, 6. The data described herein provides new insights into the complexity of Avr genes and their associated functionality and reveal that their genomic signatures can be used as accurate predictors of phenotypes for interaction with Rps genes in soybean. In embodiments, based on these genomic signatures, a multiplex PCR test has been developed that allows to characterize precisely the virulence profile of any isolate of P. sojae, overcoming the limitation of currently used phenotyping methods. This test will for example have useful applications for soybean growers and breeders by allowing the selection and deployment of Rps genes in soybean germplasm that allow a specific resistance against the virulence profiles of P. sojae present in a given field or given region. Considering that P. sojae can cause annual losses of $1-2 billion dollars worldwide (Tyler, 2007), the test will potentially result in significant loss reductions for soybean growers by preventing infection against virulent P. sojae isolates.

### Definitions

In order to provide clear and consistent understanding of the terms in the instant application, the following definitions are provided.

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented. All methods described herein can be performed in any suitable order unless otherwise indicated herein or otherwise clearly contradicted by context.

In the present description, a number of terms are extensively utilized. In order to provide a clear and consistent understanding of the specification and claims, including the scope to be given such terms, the following definitions are provided.

Nucleotide sequences are presented herein by single strand, in the 5′ to 3′ direction, from left to right, using the one-letter nucleotide symbols as commonly used in the art and in accordance with the recommendations of the IUPAC IUB Biochemical Nomenclature Commission. An “isolated nucleic acid molecule”, as is generally understood and used herein, refers to a polymer of nucleotides, and includes, but should not limited to DNA and RNA. The “isolated” nucleic acid molecule is not in its natural in vivo state, obtained by cloning or chemically synthesized. By “isolated” it is meant that a sample containing a target nucleic acid is taken from its natural milieu, but the term does not connote any degree of purification.

The use of the word “a” or “an” when used in conjunction with the term “comprising” in the claims and/or the specification may mean “one” but it is also consistent with the meaning of “one or more”, “at least one”, and “one or more than one”.

As used in the specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”.

The term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein. For example, for the recitation of numeric ranges herein, each intervening number there between with the same degree of precision is explicitly contemplated. For example, for the range of 18-20, the numbers 18, 19 and 20 are explicitly contemplated, and for the range 6.0-7.0, the number 6.0, 6.1, 6.2, 6.3, 6.4, 6.5, 6.6, 6.7, 6.8, 6.9, and 7.0 are explicitly contemplated.

The use of any and all examples, or exemplary language (e.g., “such as”) provided herein, is intended merely to better illustrate the invention and does not pose a limitation on the scope of the invention unless otherwise claimed.

Any and all combinations and sub-combinations of the embodiments and features disclosed herein are encompassed by the present invention.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

Practice of the methods, as well as preparation and use of the products and compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

As used herein, “polynucleotide” or “nucleic acid molecule” refers to a polymer of nucleotides and includes DNA (e.g., genomic DNA, cDNA), RNA molecules (e.g., mRNA), and chimeras thereof. The nucleic acid molecule can be obtained by cloning techniques or synthesized. DNA can be double-stranded or single-stranded (coding strand or non-coding strand [antisense]). Conventional deoxyribonucleic acid (DNA) and ribonucleic acid (RNA) are included in the terms “nucleic acid molecule” and “polynucleotide” as are analogs thereof (e.g., generated using nucleotide analogs, e.g., inosine or phosphorothioate nucleotides). Such nucleotide analogs can be used, for example, to prepare polynucleotides that have altered base-pairing abilities or increased resistance to nucleases. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see “The Biochemistry of the Nucleic Acids 5-36”, Adams et al., ed., 11th ed., 1992), or known derivatives of purine or pyrimidine bases (see, Cook, PCT Intl Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

As used herein, the terms “gene” and “recombinant gene” refer to nucleic acid molecules which may be isolated from chromosomal DNA, and very often include an open reading frame encoding a protein. A gene may include coding sequences, non-coding sequences, introns and regulatory sequences, as well known.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

As used herein, the term “non-conservative mutation” or “non-conservative substitution” in the context of polypeptides refers to a mutation in a polypeptide that changes an amino acid to a different amino acid with different biochemical properties (i.e., charge, hydrophobicity and/or size). Although there are many ways to classify amino acids, they are often sorted into six main groups on the basis of their structure and the general chemical characteristics of their R groups. (i) Aliphatic (Glycine, Alanine, Valine, Leucine, Isoleucine); (ii) Hydroxyl or Sulfur/Selenium-containing (also known as polar amino acids) (Serine, Cysteine, Selenocysteine, Threonine, Methionine); (iii) Cyclic (Proline); (iv) Aromatic (Phenylalanine, Tyrosine, Tryptophan); (v) Basic (Histidine, Lysine, Arginine) and (vi) Acidic and their Amide (Aspartate, Glutamate, Asparagine, Glutamine). Thus, a non-conservative substitution includes one that changes an amino acid of one group with another amino acid of another group (e.g., an aliphatic amino acid for a basic, a cyclic, an aromatic or a polar amino acid; a basic amino acid for an acidic amino acid, a negatively charged amino acid (aspartic acid or glutamic acid) for a positively charged amino acid (lysine, arginine or histidine) etc.

Conversely, a “conservative substitution” or “conservative mutations” in the context of polypeptides are mutations that change an amino acid to a different amino acid with similar biochemical properties (e.g. charge, hydrophobicity and size). For example, a leucine and isoleucine are both aliphatic, branched hydrophobes. Similarly, aspartic acid and glutamic acid are both small, negatively charged residues. Therefore, changing a leucine for an isoleucine (or vice versa) or changing an aspartic acid for a glutamic acid (or vice versa) are examples of conservative substitutions.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized, e.g. for use in eukaryotic, mammalian and/or human cells.

The term “variant” refers herein to a nucleic acid or polypeptide, which differs from a corresponding reference sequence by virtue of a mutation or modification, including an insertion, substitution, or deletion of one or more nucleotides or amino acids, as compared to its corresponding reference molecule. In an embodiment, the reference sequence is Phytophthora sojae reference genome P6497 (http://protists.ensembl.org/Phytophthora_sojae/Info/Index). Insertions and deletions are commonly collectively referred to as “indels”. In embodiments, the mutation or modification is a single nucleotide polymorphism (SNP) or an indel.

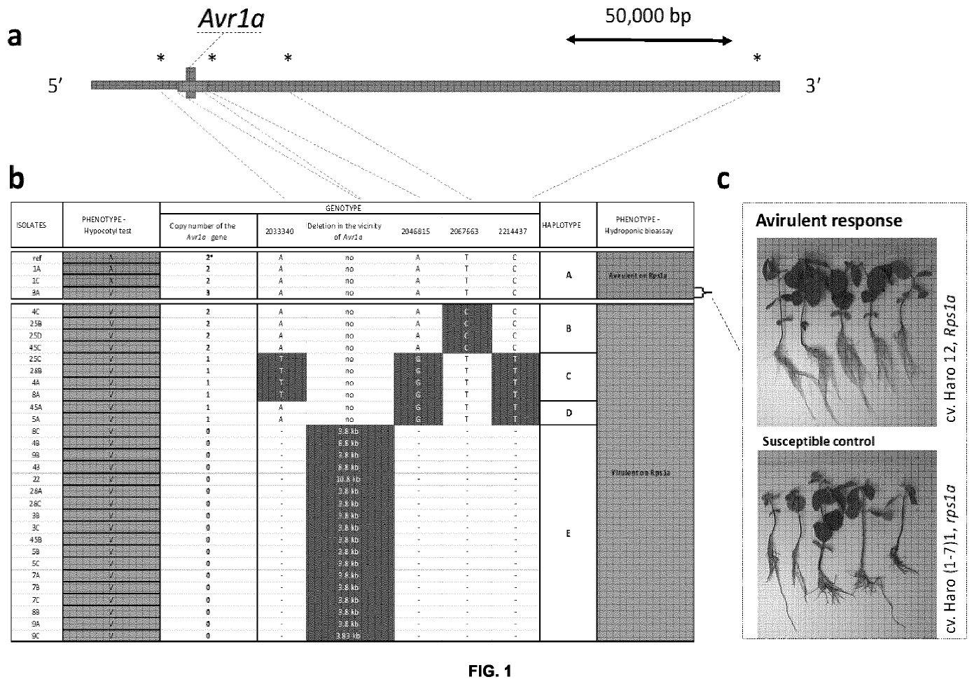

A “single nucleotide polymorphism” or “SNP” refers to a specific position in a sequence (e.g. in a genome) where there is a substitution of a nucleotide relative to a reference sequence. In embodiments the SNP is located in a coding region of a gene, in further embodiments the SNP is located in a noncoding region of a gene or in an intergenic region. In embodiments, the SNPs of the invention comprise one or more SNPs described herein, such as one or more SNPs corresponding to one or more SNPs set forth in FIGS. 1 (Avr1a), 3 (Avr1b), 5 (Avr1c), 8 (Avr1d), 10 (Avr1k), 12 (Avr3a), 14 (Avr6), 16-23, and/or Table 4 and/or 5. In embodiments, multiple SNPs may be determined simultaneously while in other embodiments SNPs may be determined separately.

In some embodiments, one or more SNPs or indels described herein may be detected or determined via the detection of a different region or SNP that is in linkage disequilibrium with the one or more SNPs or indels described herein found to be associated with virulence or avirulence of a Phytophthora pathogen. “Linkage disequilibrium” as used herein refers to the non-random association of alleles at different loci, e.g. two SNPs. Methods for measuring linkage disequilibrium are known in the art. Two such regions, e.g. SNPs, are in linkage disequilibrium if they are inherited together, and in such a case their presence is correlated with a relatively high degree of certainty.

Determining genotype, e.g. a SNP or indel, may comprise direct genotyping, e.g. by determining the identity of the nucleotide of each allele at the locus of SNP or indel, and/or indirect genotyping, e.g. by determining the identity of each allele at one or more loci that are in linkage disequilibrium with the SNP or indel in question and which allow inference of the identity of each allele at the locus of SNP in question with a substantial degree of confidence, in embodiments with a with a probability of at least 85%, 90%, 95% or 99% certainty.

In embodiments, a SNP or indel is detected through an amplification method, e.g. PCR amplification, or by nucleotide sequencing of the region comprising the SNP or indel. In embodiments a SNP or indel is detected using a probe specific for the SNP or indel, in embodiments via PCR amplification in the presence of the probe specific for the SNP or indel.

In embodiments SNPs or indels described herein may be detected using a DNA microarray. Such microarrays comprise oligonucleotides or probes bound or attached to a solid support or substrate, such as a bead, chip, glass slide or membrane. The oligonucleotides or probes may be arrayed at discrete regions on the substrate, and in turn their arrangement or organization on the substrate facilitates identification of a SNP or indel via specific probe-target interactions.

In embodiments, detection of genetic variation such as a SNP or indel is via hybridization to specific sequences which recognize the mutant and reference alleles in a nucleic acid fragment of a test sample. In embodiments, the fragment has been amplified, e.g. by PCR, and in a further embodiment labelled with a detectable label, such as a fluorescent molecule. In embodiments, the amplification reaction may be carried out on the microarray itself.

A missense mutation is a nucleotide substitution that results in a codon that codes for a different amino acid. A nonsense mutation results in the introduction of a stop codon. A readthrough mutation results in a stop codon being exchanged for an amino acid codon. Missense, nonsense and readthrough mutations are types of non-synonymous substitutions, i.e. that result in a modification of the amino acid sequence of the encoded polypeptide. A synonymous substitution in a coding sequence is one that does not modify the encoded amino acid sequence. Synonymous substitutions may also occur in non-coding regions. In embodiments, a modification, alteration or mutation described herein is synonymous or non-synonymous. In further embodiments, a modification, alteration or mutation described herein is missense, nonsense or readthrough.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

Sequence Similarity

The terms “identity” and “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length. Thus, in accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff, Proc. Natl. Acad. Sci. USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s). The skilled person will appreciate that all these different algorithms or programs will yield slightly different results but that the overall percentage identity of two sequences is not significantly altered when using different algorithms.

In a related manner, the terms “homology” or “percent homology”, refer to a similarity between two polypeptide sequences, but take into account changes between amino acids (whether conservative or not). As well known in the art, amino acids can be classified by charge, hydrophobicity, size, etc. It is also well known in the art that amino acid changes can be conservative (e.g., they do not significantly affect, or not at all, the function of the protein). A multitude of conservative changes are known in the art, Serine for threonine, isoleucine for leucine, arginine for lysine etc., Thus the term homology introduces evolutionistic notions (e.g., pressure from evolution to a retain function of essential or important regions of a sequence, while enabling a certain drift of less important regions).

The skilled person will be aware of the fact that several different computer programs are available to determine the homology between two sequences. For instance, a comparison of sequences and determination of percent identity between two sequences can be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a BLOSUM62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the Accelrys GCG software package (available at http://www.accelrys.com/products/gcg/), using a NWSgapdna.CMP matrix and a gap weight of 40, 50, 60, 70, or 80 and a length weight of 1, 2, 3, 4, 5, or 6. In another embodiment, the percent identity two amino acid or nucleotide sequence is determined using the algorithm of E. Meyers and W. Miller (CABIOS, 4:11-17 (1989) which has been incorporated into the ALIGN program (version 2.0) (available at the ALIGN Query using sequence data of the Genestream server IGH Montpellier France http://vega.igh.cnrs.fr/bin/align-guess.cgi) using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al., (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

As used herein, the expressions “corresponding to”, “corresponding to the positions”, and “at a position or positions corresponding to”, and grammatical variations thereof, refer to one or more nucleotide or amino acid positions that are determined to correspond to one another based on sequence and/or structural alignments with a specified reference gene sequence, coding sequence, or protein. For example, a position “corresponding to” an amino acid position of a given protein can be determined empirically by aligning the sequence of amino acids of that given protein with that of a polypeptide of interest that shares a level of sequence identity therewith. Corresponding positions can be determined by comparing and aligning sequences to maximize the number of matching nucleotides or residues, for example, such that identity between the sequences is greater than 95%, 96%>, 97%, 98% or 99% or more. Corresponding positions also can be based on structural alignments, for example by using computer simulated alignments of protein structure. Recitation that amino acids of a polypeptide correspond to amino acids in a disclosed sequence refers to amino acids identified upon alignment of the polypeptide with the disclosed sequence to maximize identity or homology (where conserved amino acids are aligned) using a standard alignment algorithm, such as the GAP algorithm. For example, Table 5 sets forth corresponding positions in SEQ ID NOs: 1-70 for certain SNPs and indels described herein.

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) or may contain one or more residues (including abasic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. Appropriate hybridization conditions are well known to those skilled in the art, can be predicted readily based on sequence composition and conditions, or can be determined empirically by using routine testing (see Sambrook et al., Molecular Cloning, A Laboratory Manual, 2^{nd} ed. (Cold Spring Harbor Laboratory Press, Cold Spring Harbor, N.Y., 1989) at §§ 1.90-1.91, 7.37-7.57, 9.47-9.51 and 11.47-11.57, particularly at §§ 9.50-9.51, 11.12-11.13, 11.45-11.47 and 11.55-11.57).

The present invention refers to a number of units or percentages that are often listed in sequences. For example, when referring to “at least 80%, at least 85%, at least 90% . . . ”, or “at least about 80%, at least about 85%, at least about 90% . . . ”, every single unit is not listed, for the sake of brevity. For example, some units (e.g., 81, 82, 83, 84, 85, . . . 91, 92% . . . ) may not have been specifically recited but are considered encompassed by the present invention. The non-listing of such specific units should thus be considered as within the scope of the present invention.

Nucleic acid sequences may be detected by using hybridization with a complementary sequence (e.g., oligonucleotide probes) (see U.S. Pat. No. 5,503,980 (Cantor), U.S. Pat. No. 5,202,231 (Drmanac et al.), U.S. Pat. No. 5,149,625 (Church et al.), U.S. Pat. No. 5,112,736 (Caldwell et al.), U.S. Pat. No. 5,068,176 (Vijg et al.), and U.S. Pat. No. 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ one nucleotide (see U.S. Pat. Nos. 5,837,832 and 5,861,242 (Chee et al.)).

A detection step may use any of a variety of known methods to detect the presence of nucleic acid by hybridization to an oligonucleotide probe. The types of detection methods in which probes can be used include Southern blots (DNA detection), dot or slot blots (DNA, RNA), and Northern blots (RNA detection). Labeled proteins could also be used to detect a particular nucleic acid sequence to which it binds (e.g., protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g., an array) of different probes.

A “label” refers to a molecular moiety or compound that can be detected or can lead to a detectable signal. A label is joined, directly or indirectly, for example to an oligonucleotide, a nucleic acid probe or the nucleic acid to be detected (e.g., an amplified sequence) or to a polypeptide to be detected. Direct labeling can occur through bonds or interactions that link the label to the polynucleotide or polypeptide (e.g., covalent bonds or non-covalent interactions), whereas indirect labeling can occur through the use of a “linker” or bridging moiety, such as additional nucleotides, amino acids or other chemical groups, which are either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound).

“Amplification” refers to any in vitro procedure for obtaining multiple copies (“amplicons” or “amplification products”) of a target nucleic acid sequence or its complement or fragments thereof. In vitro amplification refers to production of an amplified nucleic acid that may contain less than the complete target region sequence or its complement. In vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating nucleic acid molecules, and a replicase such as QR-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. Nos. 6,087,133 and 6,124,120 (MSDA)). Those skilled in the art will understand that the oligonucleotide primer sequences of the present invention may be readily used in any in vitro amplification method based on primer extension by a polymerase. (see generally Kwoh et al., 1990, Am. Biotechnol. Lab. 8:14 25 and (Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., 2000, Molecular Cloning—A Laboratory Manual, Third Edition, CSH Laboratories). As commonly known in the art, the oligos are designed to bind to a complementary sequence under selected conditions.

As used herein, a “primer” defines an oligonucleotide which is capable of annealing to a target sequence, thereby creating a double stranded region which can serve as an initiation point for nucleic acid synthesis under suitable conditions. The primer's 5′ region may be non-complementary to the target nucleic acid sequence and include additional bases, such as a promoter sequence (which is referred to as a “promoter primer”). Those skilled in the art will appreciate that any oligomer that can function as a primer can be modified to include a 5′ promoter sequence, and thus function as a promoter primer. Similarly, any promoter primer can serve as a primer, independent of its functional promoter sequence. Size ranges for primers include those that are about 10 to about 50 nt long and contain at least about 10 contiguous bases, or even at least 12 contiguous bases that are complementary to a region of the target nucleic acid sequence (or a complementary strand thereof). The contiguous bases are at least 80%, or at least 90%, or completely complementary to the target sequence to which the amplification oligomer binds. An amplification oligomer may optionally include modified nucleotides or analogs, or additional nucleotides that participate in an amplification reaction but are not complementary to or contained in the target nucleic acid, or template sequence. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24 and 25). The terminology “amplification pair” or “primer pair” refers herein to a pair of oligonucleotides (oligos) of the present invention, which are selected to be used together in amplifying a selected nucleic acid sequence by one of a number of types of amplification processes.

In an embodiment, the amplification reaction is a primer-dependent nucleic acid amplification reaction. The amplification reaction is allowed to proceed for a duration (e.g., number of cycles) and under conditions that generate a sufficient amount of amplification product. Most conveniently, polymerase chain reaction (PCR) will be used, although the skilled person would be aware of other techniques.

Many variations of PCR have been developed, for instance Real Time PCR (also known as quantitative PCR, qPCR), hot-start PCR, competitive PCR, and so on, and these may all be employed where appropriate to the needs of the skilled person.

In one basic embodiment using a PCR based amplification, the oligonucleotide primers are contacted with a reaction mixture containing the target sequence and free nucleotides in a suitable buffer. Thermal cycling of the resulting mixture in the presence of a DNA polymerase results in amplification of the sequence between the primers.

Optimal performance of the PCR process is influenced by choice of temperature, time at temperature, and length of time between temperatures for each step in the cycle. A typical cycling profile for PCR amplification is (a) about 5 minutes of DNA melting (denaturation) at about 95° C.; (b) about 30 seconds of DNA melting (denaturation) at about 95° C.; (c) about 30 seconds of primer annealing at about 50-65° C.; (d) about 30 seconds of primer extension at about 68° C.-72° C., preferably 72° C.; and steps (b)-(d) are repeated as many times as necessary to obtain the desired level of amplification. A final primer extension step may also be performed. The final primer extension step may be performed at about 68° C.-72° C., preferably 72° C. In certain embodiments the annealing step is performed at 50-60° C., e.g. 50-58° C., 52-58° C., 54-58° C., 53-57° C., or 53-55° C. In other embodiments the annealing step is performed at about 55° C. (e.g. 55° C.±4° C., 55° C.±3° C., 55° C.±2° C. 55° C.±1° C. or 55° C.±0.5° C.). In other embodiments the annealing step is performed at 40-60° C., e.g. 45-55° C., 46-54° C., 47-53° C., 48-52° C., or 49-51° C. In other embodiments the annealing step is performed at about 50° C. (e.g. 50° C.±4° C., 50° C.±3° C., 50° C.±2° C. 50° C.±1° C. or 50° C.±0.5° C.). The annealing step of other amplification reactions may also be performed at any of these temperatures.

In embodiments, the primers may be used, each independently, at a concentration of about 0.05 μM—about 0.50 μM, in a further embodiment 0.05 μM—about 0.40 μM, in a further embodiment 0.05 μM—about 0.30 μM, in a further embodiment 0.05 μM—about 0.20 μM, in a further embodiment 0.05 μM—about 0.15 μM, in further embodiments about 0.05 μM, 0.075 μM, 0.10 μM, 0.125 μM, 0.15 μM, 0.175 μM, or about 0.20 μM.

The detection method of the present invention may be performed with any of the standard master mixes and enzymes available. For example, commercially available PCR mix may be used, such as the QUANTITEC® PCR Master Mix (QIAGEN®) or the MAXIMA® qPCR master mix (Thermo-Scientific®). Furthermore, any conventional PCR (qPCR) instrument/system may be used, such as for example the LightCycler@ systems (Roche), SLAN® Real-Time PCR Detection Systems (Daan Diagnostics® Ltd.), Bio-Rad® real-time PCR systems, and the like.

Modifications of the basic PCR method such as qPCR (Real-Time PCR) have been developed that can provide quantitative information on the template being amplified. Numerous approaches have been taken although the two most common techniques use double-stranded DNA binding fluorescent dyes or selective fluorescent reporter probes.

Double-stranded DNA-binding fluorescent dyes, for instance SYBR Green, associate with the amplification product as it is produced and when associated the dye fluoresces. Accordingly, by measuring fluorescence after every PCR cycle, the relative amount of amplification product can be monitored in real time. Through the use of internal standards and controls, this information can be translated into quantitative data on the amount of template at the start of the reaction.

The fluorescent reporter probes used in qPCR are sequence-specific oligonucleotides, typically RNA or DNA, that have a fluorescent reporter molecule at one end and a quencher molecule at the other (e.g., the reporter molecule is at the 5′ end and a quencher molecule at the 3′ end or vice versa). The probe is designed so that the reporter is quenched by the quencher. The probe is also designed to hybridize selectively to particular regions of complementary sequence which might be in the template. If these regions are between the annealed PCR primers the polymerase, if it has exonuclease activity, will degrade (depolymerise) the bound probe as it extends the nascent nucleic acid chain it is polymerizing. This will relieve the quenching and fluorescence will rise. Accordingly, by measuring fluorescence after every PCR cycle, the relative amount of amplification product can be monitored in real time. Through the use of internal standard and controls, this information can be translated into quantitative data.

The amplification product may be detected, and amounts of amplification product can be determined by any convenient means. A vast number of techniques are routinely employed as standard laboratory techniques and the literature has descriptions of more specialized approaches. At its most simple the amplification product may be detected by visual inspection of the reaction mixture at the end of the reaction or at a desired time point. Typically, the amplification product will be resolved with the aid of a label that may be preferentially bound to the amplification product. Typically, a dye substance, e.g. a colorimetric, chromomeric fluorescent or luminescent dye (for instance ethidium bromide or SYBR green) is used. In other embodiments a labelled oligonucleotide probe that preferentially binds the amplification product is used.

In an embodiment, the amplification reaction is a multiplex amplification reaction (e.g., multiplexed PCR). “Multiplexed FOR” means a PCR wherein multiple target sequences (or a single target sequence and one or more reference sequences) are simultaneously carried out in the same reaction mixture. Usually, distinct sets of primers are employed for each sequence being amplified. Typically, the number of target sequences in a multiplex PCR is in the range of from 2 to 10, or from 2 to 8, or more typically, from 3 to 6.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. In an embodiment, the probe defined herein is a hydrolysis probe (e.g., TaqMan® probe) and comprises a fluorophore and a quencher attached thereto.

In an aspect, the present invention relates to assessing whether a plant pathogen is virulent or avirulent. In an embodiment, the pathogen is an oomycete. In a further embodiment, the pathogen is of Phytophthora spp. (mostly pathogens of dicotyledons; produces mildew), e.g., Phytophthora sojae (soya bean root and stem rot), Phytophthora infestans (potato late blight; destruction of solanaceous crops such as tomato and potato). In an embodiment the pathogen is Phytophthora sojae (soya bean root and stem rot). In embodiments, the plants of interest include vegetables, oil-seed plants and leguminous plants. In an embodiment, the plant of interest is soybean (Glycine max).

The determination of whether the plant pathogen, e.g. a Phytophthora pathogen, is virulent or avirulent is performed on the basis of the determination of the pathotype of the pathogen, e.g., the presence or absence of one or more variations, e.g. one or more indels and/or SNPs corresponding to one or more indels and/or SNPs described herein, in one or more Avr genes or a flanking region thereof, which in embodiments may be determined by directly identifying the presence or absence of one or more SNPs or indels or by indirectly identifying the presence or absence of one or more SNPs or indels by virtue of the assessment of another region or loci that is in linkage disequilibrium with the one or more SNPs or indels. In embodiments, the one or more Avr genes are one or more of Avr1a, Avr1b, Avr1c, Avr1d, Avr1k, Avr3a and Avr6. In a further embodiment, the one or more Avr genes is one or more of Avr1a, Avr1b, Avr1c, Avr1d, Avr1k and Avr6. In an embodiment, the one or more Avr genes is not Avr3a.

In embodiments, the one or more variations are one or more indels and/or SNPs corresponding to one or more indels and/or SNPs set forth in FIGS. 1 (Avr1a), 3 (Avr1b), 5 (Avr1c), 7 (Avr1d), 9 (Avr1k), 11 (Avr3a), 13 (Avr6) and/or 16-23, Table 4 and/or Table 5, and one or more discriminant positions set forth in Table 2. In embodiments, any single SNP or indel may be used to assess virulence or avirulence. In embodiments, any combination of 2 or more, SNP(s) and/or indel(s) (SNP(s), indel(s) or combination(s) thereof, e.g., 2 or more SNPs, 2 or more indels, 1 SNP+1 indel, 1 SNP+2 or more indels, 2 or more SNPs+1 indel, 2 or more SNPs+2 or more indels) may be used to assess virulence or avirulence. In further embodiments, any combination of 3 or more, 4 or more, 5 or more, 6 or more, 7 or more, 8 or more, 9 or more, or 10 or more SNP(s) and/or indel(s) may be used to assess virulence or avirulence.

The presence of a virulent pathogen, e.g. a Phytophthora pathogen, is indicative of an elevated risk of infection of the plant of interest. In such a case, in a further aspect, the present invention also relates to methods of controlling pathogen infection.

In an embodiment such methods include treatment of the plant or the agricultural area (e.g., to the soil, plants or air thereof) thereof with an antipathogen agent. In an embodiment, the antipathogen agent is a fungicide, which counters infection and disease caused by fungi or fungus-like organisms (e.g. oomycete), by specifically inhibiting or killing the fungus or fungus-like organism causing the disease. Fungicides are applied most often as liquid, but also as dust, granules and gas. In the field, they are for example applied to (1) soil, either in-furrow at planting, after planting as a soil drench (e.g., drip irrigation), or as a directed spray around the base of the plant; (2) foliage and other aboveground parts of plants via spraying; (3) in gaseous form in the air in enclosed areas such as greenhouses and covered soil. Post-harvest, they may be applied to harvested produce for example via dipping or spraying. In embodiments, the fungicide is a phosphonate fungicide, which is effective for example against oomycetes. Numerous fungicides for various pathogens and associated plant diseases are well known in the art.

In a further embodiment, methods of controlling pathogen infection include selection of a plant that is resistant to the particular plant pathogen. For example, the development of stem and root rot caused by P. sojae is determined by the gene-for-gene relationship between resistance (Rps) genes in soybean and their matching avirulence (Avr) genes in the pathogen. Thus, in the case of the identification of a virulent pathogen, e.g. a Phytophthora pathogen, or an elevated or high risk of infection of such a pathogen, a cultivar (e.g. a soybean cultivar) may be selected for planting that comprises one or more resistance (Rps) genes that confer resistance to the one or more Avr genes identified in the pathogen, thereby conferring resistance thereto.

In embodiments, such plant pathogen risk assessments may be utilized for the development and provision of resistant cultivars and seeds for planting.

The present invention further provides a collection, kit or package comprising one or more reagents for the assessment of pathogen virulence, for example one or more oligonucleotides (e.g. primers, probes) for such a use. In an embodiment, the kit or package further comprises additional reagents for assessment of pathogen virulence (e.g. buffers, solutions, PCR reagents such as polymerase). In a further embodiment, the kit or package further comprises with instructions for use, such as for assessment of pathogen virulence. In embodiments, various reagents, e.g. oligonucleotides, may be attached or bound to a solid support.

Also provided is an isolated nucleotide comprising one or more SNPs or indels described herein, in a form that is non-naturally occurring.

### MODE(S) FOR CARRYING OUT THE INVENTION

The present invention is illustrated in further details by the following non-limiting examples.

### Example 1: Methods

Plant Material and Phytophthora sojae Isolates

All P. sojae isolates used in this study (including 31 isolates of Phytophthora sojae, representing 12 different pathotype profiles (races), shown e.g. in Table 1) were sampled across Ontario (Canada). Each isolate was characterized for its pathotype/virulence profile using the hypocotyl wound-inoculation technique (Xue et al. 2015) where a set of eight differential soybean lines were used, each containing a single resistance Rps gene (Rps1a, Rps1b, Rps1c, Rps1d, Rps1k, Rps3a, Rps6 and Rps7), and ‘Williams’ (rps) as a universal susceptible check.

TABLE 1
Races and associated pathotypes of Phytophthora sojae
isolates characterized in this study, as determined
by hypocotyl wounding inoculation (Xue et al. 2015).

| | Pathotype | Number of | Isolates |
|---|---|---|---|
| Race | profile | isolates | IDs |
| 1 | 7 | 2 | 1A, 1C |
| 3 | 1a, 7 | 3 | 3A, 3B, 3C |
| 4 | 1a, 1c, 7 | 3 | 4A, 4B, 4C |
| 5 | 1a, 1c, 6, 7 | 3 | 5A, 5B, 5C |
| 7 | 1a, 3a, 6, 7 | 3 | 7A, 7B, 7C |
| 8 | 1a, 1d, 6, 7 | 3 | 8A, 8B, 8C |
| 9 | 1a, 6, 7 | 3 | 9A, 9B, 9C |
| 22 | 1a, 1c, 3a, 6, 7 | 1 | 22 |
| 25 | 1a, 1b, 1c, 1k, 7 | 3 | 25B, 25C |
| 25 + 1d | 1a, 1b, 1c, 1d, 1k, 7 | 1 | 25D |
| 28 | 1a, 1b, 1k, 7 | 3 | 28A, 28B, 28C |
| 43 | 1a, 1c, 1d, 7 | 1 | 43 |
| 45 | 1a, 1b, 1c, 1k, 6, 7 | 3 | 45A, 45B, 45C |

The isolates were first subcultured on V8 agar medium (20% clarified V8) covered with wax paper to facilitate harvest of hyphae and spores. After one week, cultures were scraped off the paper with a scalpel and placed in 1.5-ml tubes with screw caps (OMNI International inc., Kennesaw, Gerorgia, United States). The tubes were then kept in the freezer at −80° C. for 2-3 hours, and lyophilized overnight. The lyophilized samples were crushed with an Omni Bead Ruptor 24 (OMNI International). Then, the DNA was extracted from the crushed samples using the E.Z.N.A Plant DNA kit (Norcross, Ga., United-States) following the manufacturer's protocol for dried samples with slight modifications.

DNA Extraction and Sequencing

DNA was extracted for each of the 31 isolates using the E.Z.N.A. Plant DNA Kit (Omega Bio-Tek Inc., Norcross, Ga., USA). The DNA quantity and quality was assessed using a NanoDrop ND-1000 spectrophotometer (NanoDrop technologies). Each sample was normalized to 10 ng/μL for sequencing library construction using the NEBNext Ultra II DNA Library Prep Kit for Illumina (New England BioLabs Inc, Ipswich, Massachusets, USA). Library quality was determined using the Agilent 2100 Bioanalyzer (Agilent Technologies). An average fragment size of approximately 650 bp was observed among all 31 individual samples. Paired-end, 250-bp sequencing was performed on an Illumina HiSeq 2500 (CHU, Québec, Canada).

Reads alignment to the reference genome Quality of the reads obtained from sequencing were checked using FastQC (Babraham Institute, Cambridge, UK). Reads were processed using Trimmomatic (Bolger et al. 2014) to remove adapter sequences and bases with a Phred score below 20 (using the Phred+33 quality score). Trimmed reads were aligned against the Phytophthora sojae reference genome V3.0 (Tyler et al. 2006) using the Burrows-Wheeler Transform Alignment (BWA) software package v0.7.13 (Li and Durbin 2009).

Presence/Absence Polymorphisms and Copy Number Variation

To detect loss of avirulence genes in some isolates from the reference genome (presence/absence polymorphisms), we calculated the breadth of coverage for each gene, corresponding to the percentage of nucleotides with at least one mapped read (lx coverage), as per Raffaele et al. (2010). If the value of the breadth of coverage was below 80%, the gene was considered to be absent. For detection of copy number variation (CNV), we compared the average depth of coverage for each locus in every isolate and normalized the counts using the mean coverage of the genic region in every isolate.

Variant Detection

Variant calling was done using the Genome Analysis Toolkit (GATK) (DePristo et al. 2011), a variant calling pipeline based on GATK's best practices. The resulting raw vcf file was quality filtered using the vcfR package (Knaus and Grünwald 2017). For haplotype visualization, a simple visual inspection was sufficient in most cases, but a custom script developed at Université Laval was used in other cases, based on a gene-centric haplotyping process that aims to select only markers in the vicinity of a gene that are found to be in strong linkage disequilibrium (LD) (Tardivel et al. 2014).

Virulence Screening Using the Hydroponic Assay

Whenever an isolate had a phenotype predicted by the hypocotyl assay (Xue et al. 2015) discordant from the other isolates within a given haplotype, this isolate was re-phenotyped using the hydroponic assay developed by Lebreton et al. (2018), in which zoospores are inoculated directly into the hydroponic nutrient solution. For this purpose, the isolate was tested against the appropriate differential line with three to six plants for every replicate together with a susceptible control cultivar not carrying the appropriate Rps gene, a resistant control cultivar and a number of control isolates. Phenotypic responses for resistance or susceptibility were recorded at 14 days post-inoculation.

Expression Analysis

Total RNA was extracted from seven-day-old P. sojae-infected soybean roots using the Trizol reagent followed by purification using the Qiagen RNeasy Mini kit (Valencia, Calif., USA). The RNA samples were treated with DNase I enzyme to remove any contaminating DNA. A total of 3 μg RNA from each sample were used to synthesize single-stranded cDNA using oligo-dT primed reverse transcription and Superscript 11 reverse transcriptase (Invitrogen™ Carlsbad, Calif., USA) following the manufacturer's protocol. Primers for the quantitative reverse transcription PCR (qPCR) analysis were designed using PrimerQuest tool and the intercalating dyes design option (Coralville, Iowa, USA). Four biological replications were used for the expression analysis. Expression analysis was carried out for Avr genes in both avirulent and virulent isolates using the iQ™ SYBR® Green Supermix (Bio-Rad, Hercules, Calif., USA) and a MIC qPCR thermocycler machine (Bio Molecular Systems, Upper Coomera, Queensland, Australia). The PCR profile consisted of an initial activation of 95° C. for 3 min, followed by 40 cycles of 95° C. for 15 s and 60° C. for 45 sec. After cycling, dissociation curve analysis (with an initial hold of 95° C. for 10 sec followed by a subsequent temperature increase from 55 to 95° C. at 0.5° C./s) was performed to confirm the absence of nonspecific amplification. Actin was used as a constitutively expressed reference transcript. Relative quantification analysis was performed using the MIC-qPCR software which uses the LinRegPCR method developed by Ruijter et al. (2009) and the Relative Expression Software Tool (REST) for statistical significance (Pfaff) et al. 2002).

Confirmation of Haplotype Variation Using Sanger Sequencing

The isolates were freshly grown in V8 agar media for seven days under controlled conditions followed by DNA extraction. Regions spanning the Avr genes were amplified using specific sets of primers. The PCR profile was initial denaturation at 98° C. for 30 sec followed by 35 cycles of denaturation at 98° C. for 10 sec, annealing at 60° C. for 30 sec and extension at 72° C. for 2 min, and the final extension at 72° C. for 10 min. The PCR products were purified using the QIAquick PCR purification kit (Qiagen, Valencia, Calif., USA) followed by sequencing on an Applied Biosystems sequencer (ABI 3730x1 DNA Analyze) located at the CHU, Quebec, Canada. The sequencing results were analyzed using the SeqMan program implemented in the DNASTAR Lasergene software (Madison, Wis., USA).

Sequence Variations and Allele-Specific Primer Design

For designing allele-specific primers, the discriminant variations in the sequences of the different Avr genes of 31 isolates were studied and identified based on the genomic sequences available in the NCBI SRA repository, under the bioproject PRJNA434589 as reported by Arsenault-Labrecque et al. (2018). In all cases, we sought to obtain amplicons only from the avirulent allele(s) and of different sizes such that primers could be used in a multiplex assay and the amplicons easily resolved via gel electrophoresis. Discriminant variations most convenient for marker development were selected to design the primer pairs for the seven Avr genes under study (Avr1a, 1b, 1c, 1d, 1k, 3a and 6). In cases where deletions were present, at least one primer was positioned in the deletion such that the avirulent allele (i.e. without the deletion) could be amplified. If only SNPs differentiated the virulent and avirulent alleles, primers were designed in such a way that these variant positions were located at the 3′ extremity to maximize the specificity of amplification. Regions with two or more SNPs were preferentially selected to increase the allelic specificity. The primers were then synthesized by Thermo-Fisher Scientific (Waltham, Mass., USA). The details of the nine pairs of primers are presented in Table 1.

Primer Design for Multiplex PCR

Primers were designed based on the different haplotypes from the seven avirulence genes (Avr1a, Avr1b, Avr1c, Avr1d, Avr1k, Avr3a and Avr6) of P. sojae, interacting with the seven resistance genes, respectively Rps1a, Rps1b, Rps1c, Rps1d, Rps1k, Rps3a and Rps6, of soybean. If an indel was present in the sequence of the avirulence gene, primers were designed in this indel to discriminate the avirulent isolates from the virulent ones. If no indel was present, at least two neighboring SNPs were used to design the primers to increase the specificity of the primers. DNA from the 31 Phytophthora sojae isolates was extracted and used to test the designed primers.

Each primer was first tested in an individual PCR reaction to validate its specificity. The PCR reaction was carried out with a reaction volume of 20 μl and each primer was diluted at a concentration of 0.25 μM. The One Taq NEB (New England Biolabs, Ipswich, Mass., USA) was used as an enzyme at 0.025 U/μl with 2 μl of DNA extracted from P. sojae at a concentration of 10 ng/μl, 5× One Taq Standard reaction Buffer (New England Biolabs, Ipswich, Mass., USA), 0.2 mM of dNTPs and 2.5% of DMSO. For each avirulence gene, P. sojae isolates avirulent and virulent to the corresponding Rps genes (see Table 1) were chosen to characterize the specificity of each primer. The PCR reaction conditions were as follows: an initial denaturation at 94° C. for 5 minutes, followed by 30 cycles of denaturation at 94° C. for 30 seconds, annealing at 60° C. for 30 seconds, elongation at 68° C. for 1 minute, and a final elongation at 68° C. for 5 minutes. The migration of the PCR samples was performed on 1.5% agarose gel with 1×TAE buffer, containing 2,5 μl/mL of SYBR safe DNA gel stain (Invitrogen, Carlsbad, Calif., USA). A ladder of 1-1000 kb was used. DNA fragment analysis was also performed using a QIAxcel Advanced System on a DNA high resolution cartridge, based on method OH500 with alignment markers of 15 and 3000 bp according to the manufacturer's instructions (Qiagen, Hilden, Germany). A PCR was performed on each of the 31 isolates of P. sojae with a known pathotype to validate that the presence of the expected amplicon was associated with an avirulent response.

Multiplex PCR Optimization

Once the specificity of each primer was validated, all the primers were tested together in a multiplex PCR, to check for compatibility of all the primers in a unique PCR reaction. Different parameters were tested to optimize the reaction. First of all, the concentration of each primer was adjusted according to the intensity of the bands to obtain clear bands for all the avirulence genes detected. The number of cycles for the reaction was increased from 30 to 40 cycles to obtain more distinct bands. Furthermore, a temperature gradient was tested to determine that the optimal temperature was 55° C. The dNTPs concentration was increased to 2.5 mM. The concentrations of the other PCR products remained the same. The final PCR products were analyzed by QIAxcel Advanced System (Qiagen, Hilden, Germany).

Following optimization of primer concentration, annealing temperature and dNTP concentration, primers were mixed together in a single PCR reaction to check their compatibility in a multiplex PCR. It was found that the primers amplifying the Avr1c gene were not compatible with the other primers since, when put together, primer dimers were formed. Attempts to design alternative sets of primers were unsuccessful, so it was decided that the primers for Avr1c would be used in a separate assay in parallel with the multiplex assay. The multiplex PCR therefore contains the following eight primer sets: Avr1a-indel, Avr1a-snp1, Avr1a-snp2, Avr1b, Avr1d, Avr1k, Avr3a and Avr6.

The optimal number of cycles for the reaction was 40 cycles. Furthermore, a temperature gradient revealed that the temperature allowing obtaining the darkest and most distinct bands was 55° C. for the multiplex PCR reaction and 60° C. for the uniplex PCR. The dNTP concentration chosen was 0.25 mM. The final PCR products were analyzed with the QIAxcel Advanced System (Qiagen, Hilden, Germany).

The PCR reactions were carried out in a reaction volume of 20 μl. Each primer was diluted at the optimal concentration detailed in Table 1. The One Taq NEB (New England Biolabs, Ipswich, Mass., Etats-Unis) was used at 0.025 U/μl with 2 μl of DNA at a concentration of 10 ng/μl, 5× One Taq Standard reaction buffer (New England Biolabs, Ipswich, Mass., USA), 0.25 mM of dNTPs and 2.5% of DMSO (Sigma, Saint-Louis, Mo., United States). The multiplex PCR conditions consisted in an initial denaturation at 94° C. for 5 min, followed by 40 cycles of denaturation at 94° C. for 30 sec, annealing at 55° C. for 30 sec, elongation at 68° C. for 1 min, and a final elongation at 68° C. for 5 min. For the uniplex PCR reaction (Avr1c), the conditions consisted in an initial denaturation at 94° C. for 5 min, followed by 30 cycles of denaturation at 94° C. for 30 sec, annealing at 60° C. for 30 sec, elongation at 68° C. for 1 min, and a final elongation at 68° C. for 5 min.

Detection Limits of the Multiplex PCR

To determine the lowest concentration of DNA at which the multiplex and the uniplex PCR worked, dilutions from 0.01 μg to 20 ng were tested with the two PCR conditions described above. It was determined that the PCR multiplex could detect a DNA concentration of up to 0.2 ng, while the primers tested individually could detect a DNA concentration of 0.2 ρg.

Specificity of the Molecular Tool and Phenotyping

Once the multiplex PCR conditions were optimized, the 31 isolates with known haplotypes, previously sequenced by Arsenault-Labrecque et al. (2018), were analysed to test the efficiency of the molecular tool. Subsequently, 15 uncharacterized isolates were both with the multiplex PCR and phenotyped using the hydroponic assay developed by Lebreton et al. (2018). For the assay, zoospores were inoculated into a hydroponic system containing a nutrient solution diluted in water. Seven differential soybean lines were grown in the hydroponic system with a susceptible control (cultivar Harosoy), and the virulence profile of the isolate tested was determined on the basis of which Rps genes resulted in immunity. Phenotypic responses (resistance or susceptibility) were recorded 14 days post inoculation. Phenotyping results were then compared to results obtained with the multiplex PCR assay.

Multiplex PCR Validation

Once the PCR conditions were optimized, the 31 isolates with known haplotypes (Table 1) were analysed to test the efficacy of the molecular tool. Subsequently, isolates with unknown haplotypes were analysed and the results were compared with the phenotyping results performed with the hydroponic essay (Lebreton et al., 2018).

### Example 2: Results

Sequencing and Mapping

A total of 852,950,094 reads were obtained from paired-end sequencing of the 31 P. sojae isolates on the Illumina HiSeq 2500 sequencer. The number of sorted raw sequence reads per isolate ranged from 15 to 52 M reads with an average of 27 M reads per isolate, with a mean Phred-score of 32.4. Reads were processed using Trimmomatic and the processed reads were mapped to the reference genome. For every isolate, more than 96% of the reads were accurately mapped to the reference genome and the mean depth of coverage was 68×.

Coverage, Distribution and Predicted Functional Impact of SNPs

The HaplotypeCaller pipeline from GATK retained 260,871 variants among the 31 isolates. Stringent filtering of the variants based on sequence depth and mapping quality using vcfR retained a total of 204,944 high-quality variants. Variant analysis with SnpEff tool (Cingolani et al. 2012) identified 172,143 single nucleotide polymorphisms (SNPs), 14,627 insertions and 18,174 small indels in the total number of variants. Variants in coding regions were categorized as synonymous and non-synonymous substitutions; 61.1% of the SNPs resulted in a codon that codes for a different amino acid (missense mutation; 59.5%) or the introduction of a stop codon (nonsense mutation: 1.6%), whereas the remaining 38.9% of the SNPs were considered to be synonymous mutations. Links among the seven Avr genes were then further investigated on the basis of haplotype analysis.

Haplotypes for Avr1a

For all 31 isolates, CNV was analyzed based on depth of coverage and, for Avr1a, it ranged between zero and three copies (FIG. 1b). Among isolates with zero copy, all were virulent on Rps1a. For the remaining isolates, no SNPs or indels were observed within the coding region of Avr1a (FIG. 1a). However, we observed SNPs flanking Avr1a that were in high LD (R² 0.7) and defined four distinct haplotypes (FIG. 1b). Additional variants were also found but did not offer a higher level of discrimination. All isolates sharing three of these (B, C and D) were virulent on Rps1a while among isolates with haplotype A, all but isolate 3A were incompatible based on the hypocotyl assay. After re-phenotyping this isolate with the hydroponic bioassay, it was characterized as being unable to infect the differential carrying Rps1a confirming that haplotype A was the only one associated with an incompatible interaction with Rps1a (FIG. 1c).

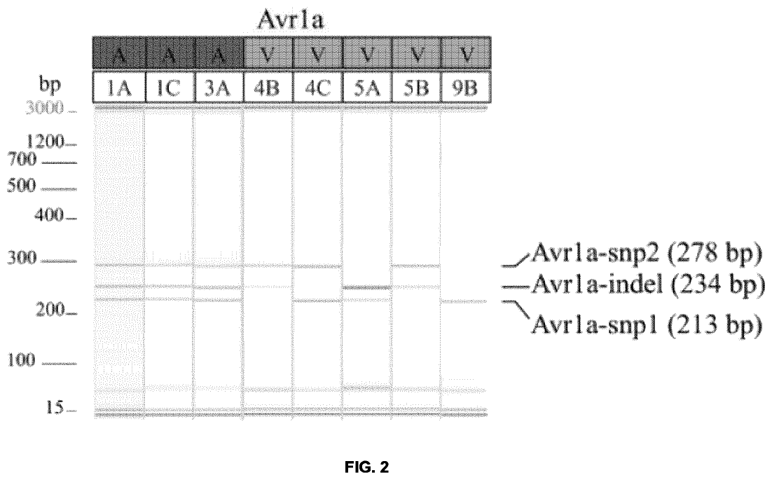

Based on these haplotypes, three discriminant primers were designed to get an amplification when isolates are avirulent to Rps1a. The first pair of primers is located within the Avr1a gene (PHYSOscaffold_7:2042431-2042664), allowing to first identify virulent isolates showing a complete deletion of the gene (haplotype E). The primer sequences are presented in Table 2. The product size obtained is 234 bp as shown in FIG. 2.

TABLE 2
Primer sequences for each avirulence gene and its product size following amplification

| | Amplicon | Product | | |
|---|---|---|---|---|
| Primers | position | size | Forward^{†} | Reverse^{†} |
| Avr1a-indel | Scaffold_7: | 234 | GAAAGTGGACGGATATTTTCAAC | CAAGGACGGACTGGTACAGA |
| | 2,042,431- | | (SEQ ID NO: 71) | (SEQ ID NO: 72) |
| | 2,042,664 | | | |
| Avr1a-snp1 | Scaffold_7: | 213 | CTTAGTGTGCACCAACAGCCA | ACCACACTTCACGGAGCATT |
| | 2,263,667- | | (SEQ ID NO: 73) | (SEQ ID NO: 74) |
| | 2,263,879 | | | |
| Avr1a-snp2 | Scaffold_7: | 278 | GCTTTTCATCCAACGCTCAT | AATGATTGGCGGCAGATC |
| | 1,799,519- | | (SEQ ID NO: 75) | (SEQ ID NO: 76) |
| | 1,799,796 | | | |
| Avr1b | Scaffold_6: | 403 | AAGGGGTACAGCCTGGATAAG | CTTGCGCTGTGAAGTGTCAT |
| | 3,146,464- | | (SEQ ID NO: 77) | (SEQ ID NO: 78) |
| | 3,146,866 | | | |
| Avr1c | Scaffold_7: | 802 | CGGCAGAAGTTCTGGAAGA | GCCTTCCTTTGTCAGATTCG |
| | 2,046,020- | | (SEQ ID NO: 79) | (SEQ ID NO: 80) |
| | 2,046,821 | | | |
| Avr1d | Scaffold_5: | 497 | CACGAGCAATGTCCTGTACG | CGAGCGTCCGATTTATAACTGG |
| | 5,919,385- | | (SEQ ID NO: 81) | (SEQ ID NO: 82) |
| | 5,919,881 | | | |
| Avr1k | Scaffold_6: | 303 | CTGTTCAGAAACTTCCGGTGC | CATGAAAAAGTCGGGGTTTG |
| | 3,142,499- | | (SEQ ID NO: 83) | (SEQ ID NO: 84) |
| | 3,142,801 | | | |
| Avr3a | Scaffold_9: | 607 | CTAGGCAAAGATGTCACCTG | ATCATGGCAAGCACCAATCT |
| | 615,324- | | (SEQ ID NO: 85) | (SEQ ID NO: 86) |
| | 615,930 | | | |
| Avr6 | Scaffold_4: | 726 | GTCGTGCTGCATACTCTTGG | CAAGCTTGAGGCTCTGTGCT |
| | 7,223,071- | | (SEQ ID NO: 87) | (SEQ ID NO: 88) |
| | 7,223,796 | | | |

^{†}Underlined nucleotides indicate discriminant nucleotide positions used to design the primers

To discriminate virulent from avirulent isolates with the remaining haplotypes (A vs B, C and D), two other pairs of primers were designed. These primers were based on SNPs 2046815 and 2067663, allowing to get an amplification when haplotype A is present, identifying the avirulent isolates only. These two SNPs were chosen because of LD with SNPs 2046815 and 2067663 and the presence of neighboring SNPs, allowing more specificity. These primers are located in the vicinity of the Avr1a gene (PHYSOscaffold_7: 2263667-2263879 and PHYSOscaffold_7: 1799519-1799796). The product size obtained is 213 bp for the first pair of primers and 278 bp for the second one, as shown in FIG. 2. The sequence of each primer is shown in Table 2.

Haplotypes for Avr1b

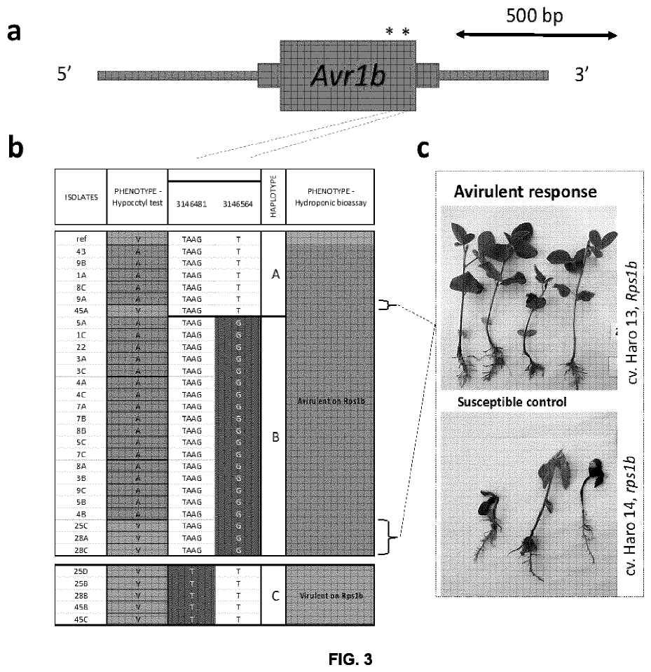

No CNVs or deletions were observed for Avr1b (FIG. 3a). Within the coding region of the gene, 17 variants were observed: 14 missense variants (SNPs), two small indels of three nucleotides each and one synonymous SNP. None of these variants were predicted to have a high functional impact. Based on the LD between these variants, two tag variants were retained and defined three haplotypes (FIG. 3b). Most isolates of haplotypes A and B were avirulent while all isolates with haplotype C were virulent. Among haplotypes A and B, four isolates with a discordant phenotype were re-tested with the hydroponic assay and were found to be avirulent to Rps1b (FIG. 3c), confirming haplotypes A and B as being consistently associated with an incompatible interaction with Rps1b (FIG. 3b). To verify that the genotype of these four isolates had not changed over time, we re-sequenced the Avr1b region of these isolates together with representative isolates from each haplotype group and confirmed the same mutations. Curiously, the isolate used for the reference genome (P6497) that is reported as virulent to Rps1b has a genotype associated with incompatibility in our study (haplotype A; FIG. 3b).

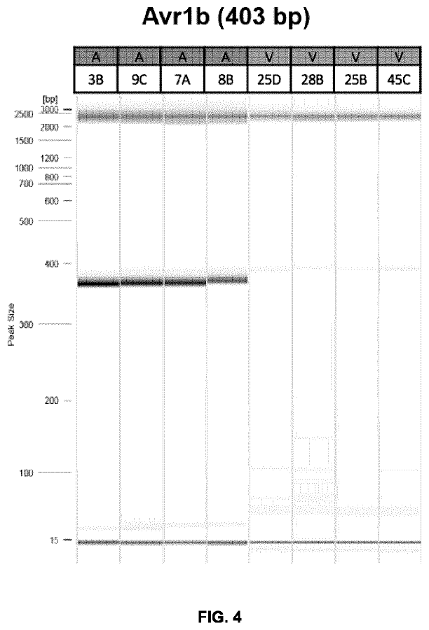

Based on the discriminant haplotypes between the avirulent and virulent isolates (A and B vs C), a pair of primers was designed within the two indels of four nucleotides to get an amplification when the isolates are avirulent (PHYSOscaffold_6:3146464-3146866). Their sequences are shown in Table 2 and the product size is 403 pb. The result of the amplification is shown in FIG. 4.

Haplotypes for Avr1c

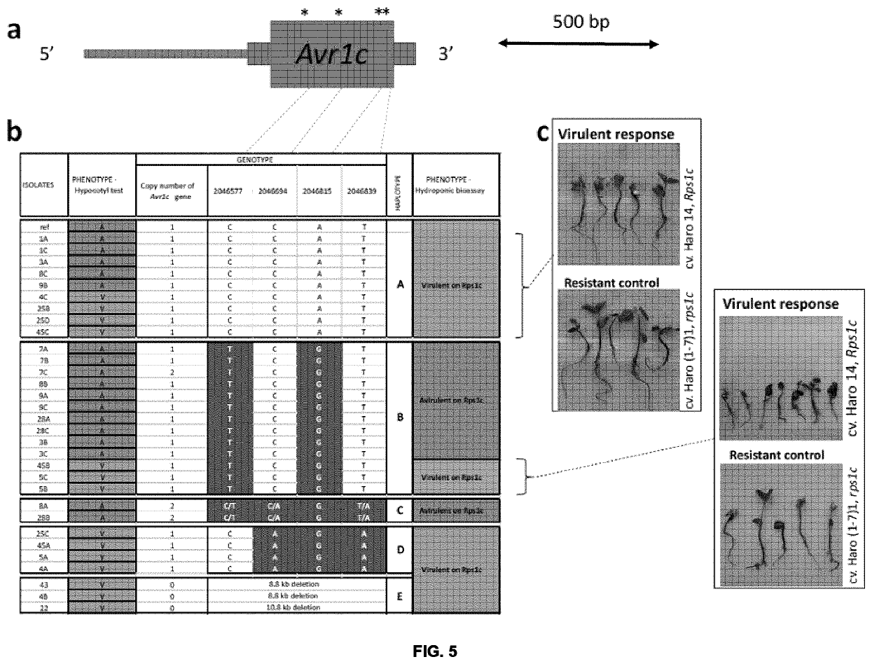

Copy number variation was observed for Avr1c; complete deletion of the Avr1c gene was observed in three isolates while others presented one or two copies of the gene (FIG. 5b). Interestingly, this deletion is the same reported earlier for the Avr1a gene that immediately flanks Avr1c (FIG. 5b and FIG. 1b). The remaining isolates presented a total of 24 variants within the coding region of the gene; two were synonymous while the rest were missense mutations, none of which being predicted to have a high functional impact. After removal of redundant markers (based on LD), a total of four tag variants defined four haplotypes (A to D; FIG. 5b). Haplotypes C and D were shared by isolates that had a consistent phenotype, avirulent and virulent, respectively (FIG. 5b). Haplotype C was also the only haplotype to present a majority of heterozygous SNPs. In contrast, haplotype A was shared by five isolates previously phenotyped as avirulent to Rps1c and four phenotyped as being virulent. All nine isolates were re-phenotyped in the hydroponic assay and the results showed a clear association with virulence to Rps1c (FIG. 5c). For haplotype B, most isolates were phenotyped as avirulent to Rps1c, with the exception of three isolates (5B, 5C and 45B) originally labelled as virulent. Variants within a 1-kb upstream or downstream region of the gene could not define new haplotypes for these three outliers. These three isolates were re-phenotyped using the hydroponic bioassay and were still characterized as virulent (FIG. 5c). To further investigate the cause of this discrepancy, the Avr1c region for representative isolates from each haplotype group, including initial outliers from haplotype A, were re-sequenced using Sanger sequencing and confirmed the same mutations.

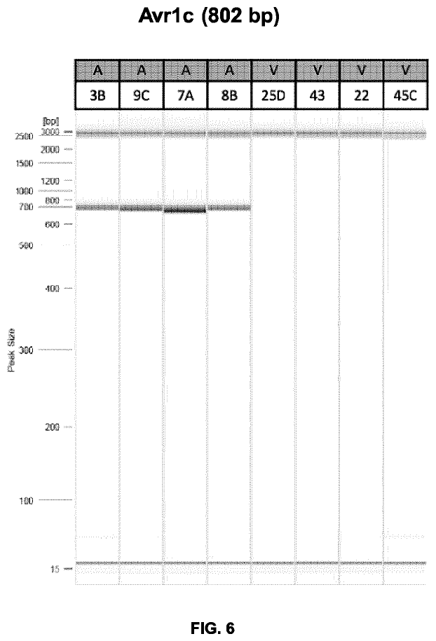

To discriminate the avirulent haplotypes B and C from the virulent haplotypes A and D (FIG. 5) for the Avr1c gene, two SNPs (U.S. Pat. Nos. 2,046,037, 2,046,038) were used at the 3′ extremity of the forward primers and four other SNPs (U.S. Pat. Nos. 2,046,815, 2,046,817, 2,046,819, 2,046,821) were used in the 5′ extremity of the reverse primer. The primer sequences are shown in Table 2 and their product size is 802 bp (PHYSOscaffold_7:2046020-2046821). FIG. 6 shows the specificity of these primers to discriminate avirulent from virulent isolates.

Haplotypes for Avr1d

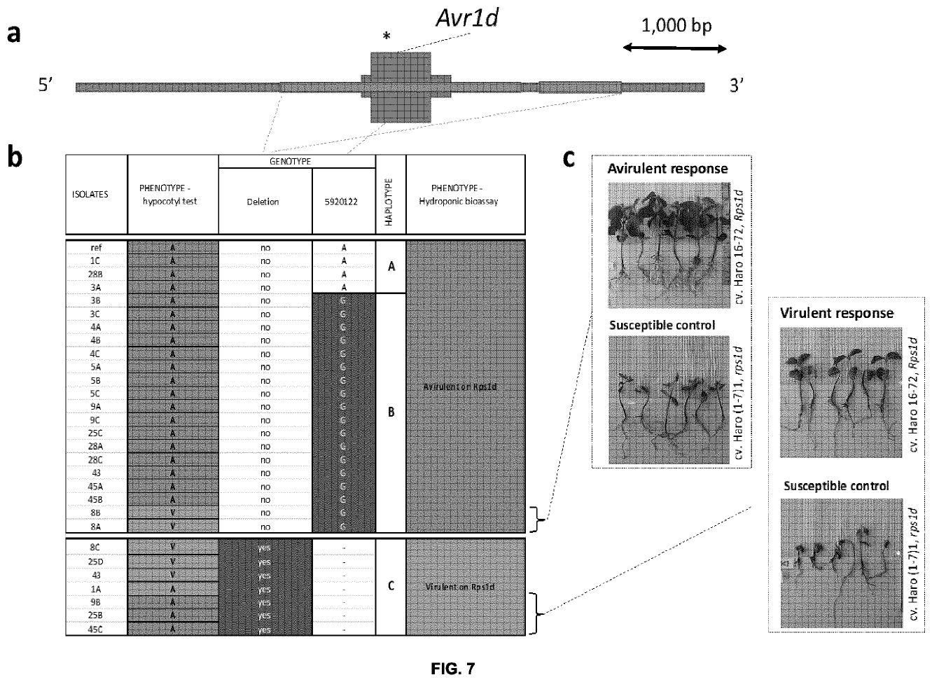

A complete deletion of the Avr1d gene was observed for seven isolates (FIG. 7b). The deletion encompassed both the upstream and downstream regions of the gene for a total deletion size of 2.3 kb, with another upstream deletion of 0.8 kb, separated by a segment of 177 bp (FIG. 7a). The remaining isolates presented one copy of the gene and 21 variants were observed within the coding region: one was synonymous while the others were missense variants, none of which were predicted to have a high functional impact. Based on LD, one tag variant was retained and two haplotypes (A and B) could be defined. Genomic data coincided with the original phenotypes based on the hypocotyl assay in 25 out of 31 interactions. However, from the original phenotyping by Xue et al. (2015), two isolates predicted to be avirulent based on the genotype were phenotyped as virulent and four isolates predicted as virulent were phenotyped as avirulent. When these isolates were phenotyped with the hydroponic assay, all the isolates with a predicted genotype of virulence were consistently associated with virulence while the isolate expected to be avirulent based on the haplotype was phenotypically avirulent, confirming that deletion of Avr1d is consistently linked to virulence (FIG. 7).

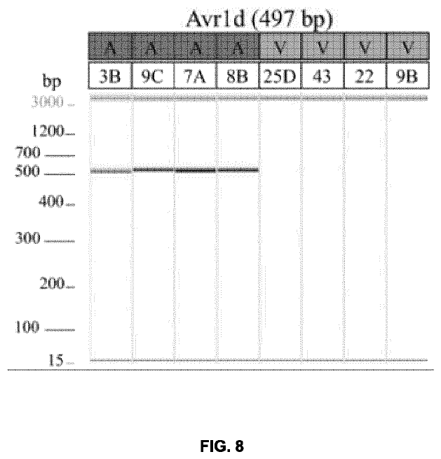

To discriminate avirulent isolates from virulent ones (haplotypes A and B vs C), a pair of primers was designed in the Avr1d gene to get an amplification in presence of an avirulent isolates and no amplification when isolates are virulent, due to the complete deletion of the gene for those isolates (PHYSOscaffold_5:5919385-5919881). The primer sequences are shown in Table 2 and the amplification is presented in FIG. 8. The product size of the amplification is 497 bp.

Haplotypes for Avr1k

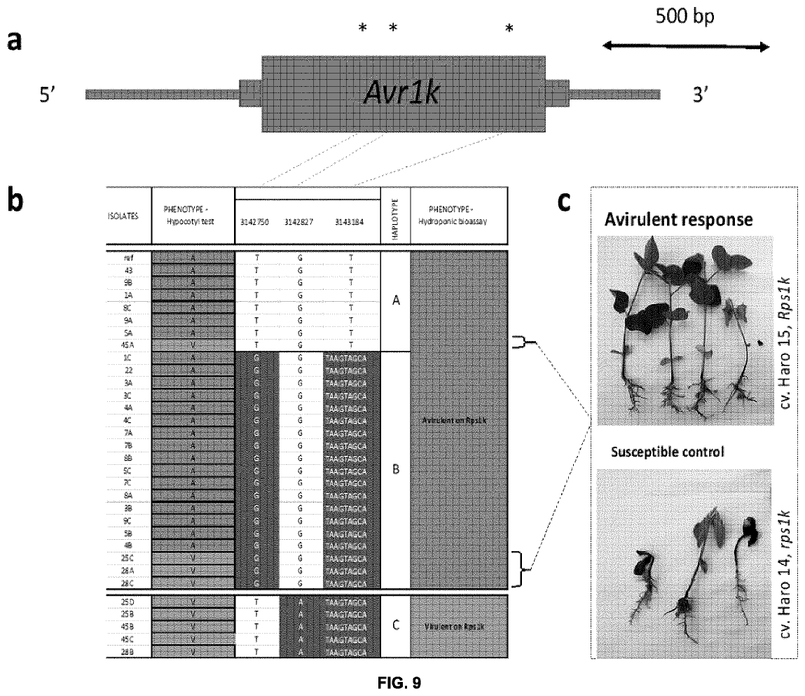

No CNVs or deletions were observed for Avr1k (FIG. 9). Inside the genic region, 16 variants were found: one synonymous variant, 14 missense variants and one deletion of eight nucleotides causing a frameshift in the ORF and leading to a premature stop codon towards the 3′ end of the gene. This latter variant is the only one considered to have a high impact on the functionality of the gene. The three tag variants within the gene (based on LD) formed three distinct haplotypes (FIG. 9b). As observed previously for Avr1b, the first two haplotypes (A and B) contained all the isolates avirulent to Rps1k plus four isolates previously phenotyped as virulent to Rps1k with the hypocotyl test. Interestingly, the exact same outliers gave an initial phenotype of virulence with Avr1b. To verify that the genotype of these outliers had not changed over time, the Avr1k gene region was re-sequenced for these isolates and showed the same mutations as observed by WGS. Haplotype C only contained isolates virulent to Rps1k. Re-phenotyping of the four outliers confirmed their incompatibility with Rps1k as shown in FIG. 9c. The eight-nucleotide frameshift mutation leading to an early stop codon was found in both haplotypes B and C, although the former was associated with an avirulent phenotype and the latter with a virulent one.

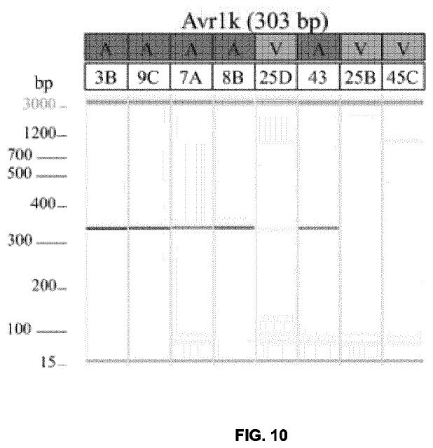

Based on avirulent haplotype A and B, a pair of primers was designed using two SNPs, based on the haplotype of SNP 3142827. The two SNPs were separated by six nucleotides and were discriminant between the avirulent and virulent isolates. The primer sequence is shown on Table 2. The position of the primers is on PHYSOscaffold_6: 3142499-3142801 and they allow to discriminate the avirulent isolates (haplotype A and B) from the virulent ones (haplotype C). The results of the amplification are shown in FIG. 10 and the product size is 303 bp. The sequences for each primer are shown in Table 2.

Haplotypes for Avr3a

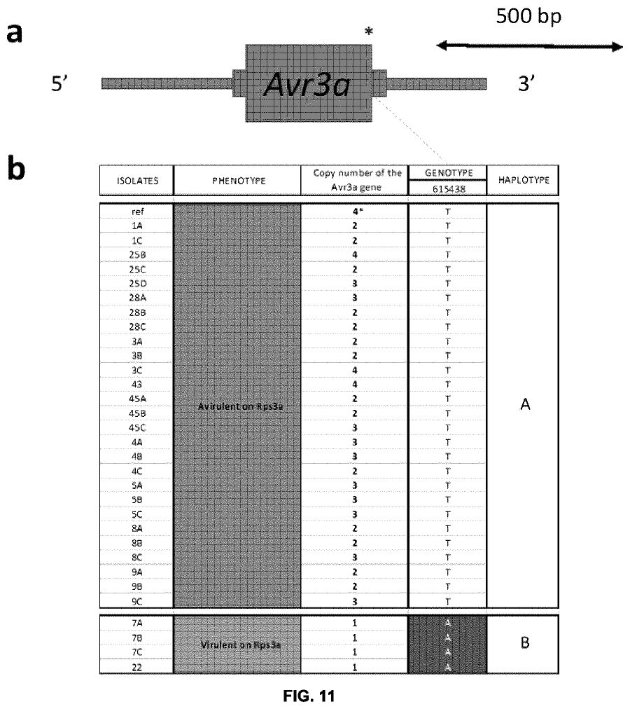

Copy number variation was observed between isolates, ranging from one to four copies; all isolates virulent to Rps3a contained one copy of the gene, while all avirulent isolates had two to four copies (FIG. 11b). Furthermore, we observed 15 variants in the coding region of the Avr3a gene, including one inframe deletion of six nucleotides and 14 SNPs, of which two were synonymous variants, 11 were missense variants, and one caused the loss of the stop codon. Only the latter variant is considered to have a high impact on the functionality of the gene. All those variants were homozygous suggesting that for isolates with multiple copies of the Avr3a gene, every copy shares the same allele. Based on the retained tag variant, two distinct haplotypes were observed. Haplotype A was consistently associated with an incompatible interaction with Rps3a while haplotype B was associated with a compatible one (FIG. 11b).

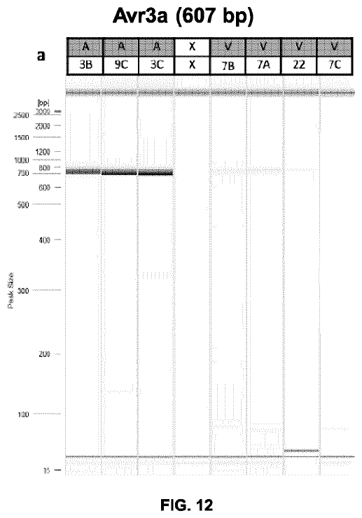

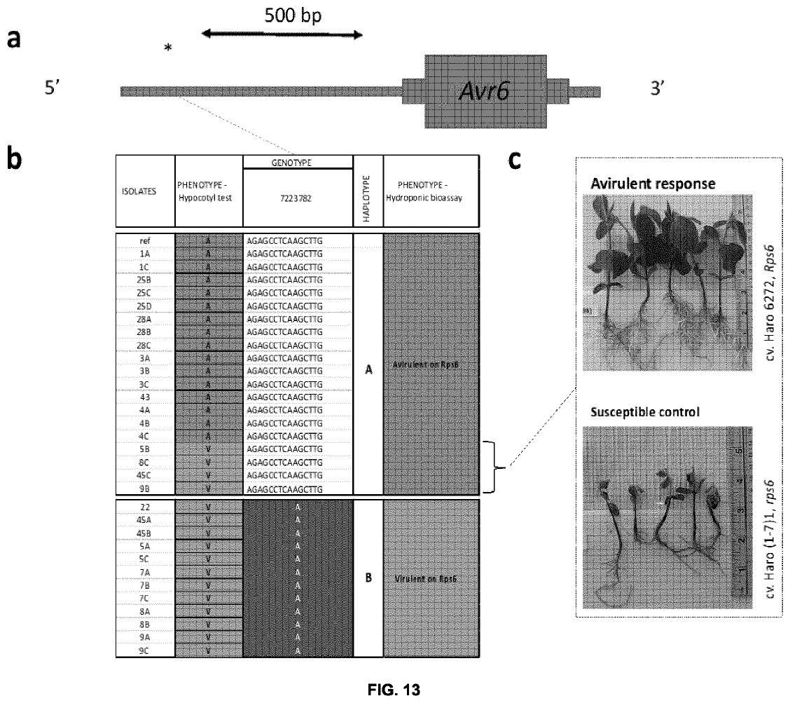

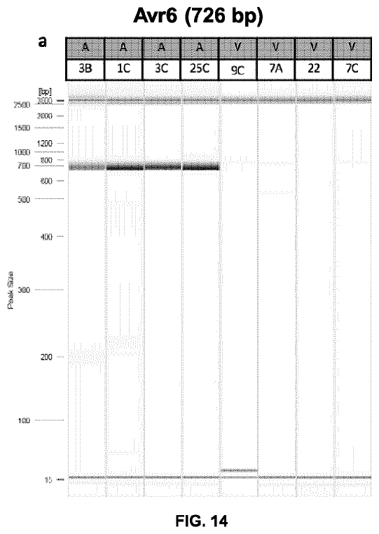

Based on these results, a pair of primers was designed based on the indel of six nucleotides to discriminate haplotype A (avirulent) from haplotype B (virulent). This way, all the avirulent isolates will amplify because of the presence of the six nucleotides in the sequence of the forward primer. The primers position is on PHYSOscaffold_9: 615324-615930 and the product size is 607 bp long as shown in FIG. 12. The sequences of each primer is shown in Table 2.

Haplotypes for Avr6

No CNVs or deletions were observed for the Avr6 gene (FIG. 13a). Furthermore, no variants were found within the coding region of Avr6, but five were found in the upstream region of the gene. From these, four were SNPs, and one was a deletion of 15 nucleotides, but none of them were predicted to have a high functional impact. A visual inspection of these variants revealed two distinct haplotypes, represented by one tag variant in FIG. 13b. All isolates incompatible with Rps6 based on the hypocotyl test were associated with haplotype A, as well as four isolates initially phenotyped as virulent. These four isolates were found to be avirulent to Rps6 via the hydroponic assay (FIG. 13c). Isolates corresponding to haplotype B were consistently associated with a compatible interaction.

With the presence of an indel of 15 bp in the Avr6 gene which is discriminant between the avirulent (haplotype A) and virulent isolates (haplotype B), a pair of primers containing these 15 nucleotides was designed. In this way, amplification was obtained when isolates are avirulent. The amplification has a product size of 726 bp (FIG. 14) and is on PHYSOscaffold_4: 7223071-7223796. Their sequences are shown in Table 2.

Gene-Specific PCR-Based Markers for Seven Avr Genes in Phytophthora sojae

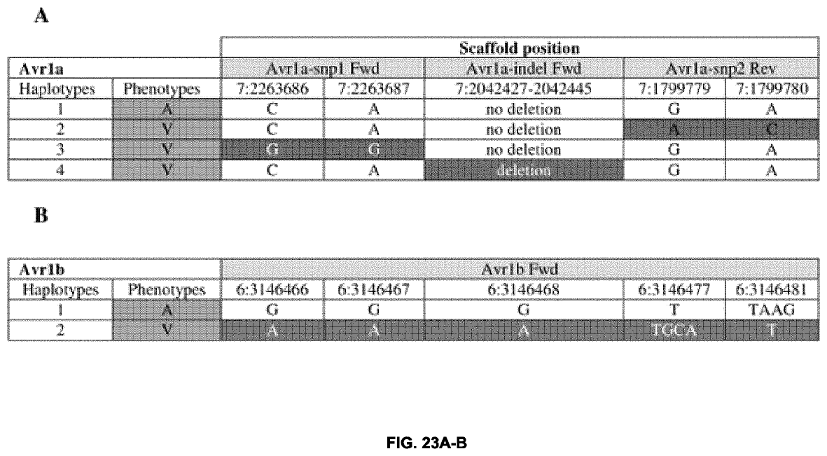

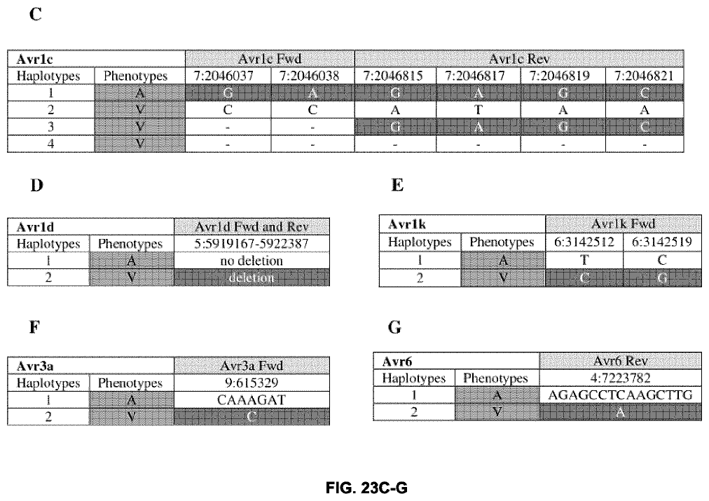

For all seven Avr genes under study, all primers were designed to amplify sequences associated with the avirulent allele of the genes (FIG. 23). In cases where the discriminant variants were located outside of the coding region, the primers were developed based on the specific haplotype linked to the avirulent allele. The positions of all the amplified regions are shown in Table 2.

For Avr1a gene, multiple variants distinguished alleles conferring virulence or avirulence on soybean lines carrying the Rps1a gene (FIG. 23A). One such variant was an 18-bp deletion conferring virulence to all isolates carrying it. For those isolates lacking the deletion, they were distinguished on the basis of two adjacent SNPs, found in two separate regions (Avr1a-snp1 and Avr1a-snp2), associated with a difference in virulence.

In the case of Avr1b, a combination of SNPs and indels, located within 15 bp of each other, was found to discriminate the avirulence allele (FIG. 23B). A primer was thus designed in that region to encompass all five variants. The Avr1c avirulent allele could be discriminated from the virulent form on the basis of two SNPs situated at the 3′ end of the forward primer, and four SNPs positioned at the 5′ end of the reverse primer (FIG. 23C). This design allowed to target specifically the avirulent haplotypes against several other haplotypes linked to virulence.

In the case of P. sojae isolates carrying Avr1d, they were easily distinguished from those with pathotype 1d on the basis of a complete deletion of the gene (FIG. 23D). Primers were thus simply designed to amplify a region within the gene.

For Avr1k, two SNPs were selected within 7 bp of each other that discriminated the avirulent allele from the virulent one to design the primers (FIG. 23E).

Based on two distinct haplotypes, the avirulent allele of Avr3a presented an extra sequence of six nucleotides (FIG. 23F). This area was therefore selected to design discriminate primers.

Finally, a 15-bp deletion upstream of Avr6 was consistently observed in all virulent isolates (FIG. 23G). As it was consistently associated with a phenotype of virulence, it was used for primer design.

Uniplex PCR Amplification and Specificity

The results showed that successful amplification of the functional version of the Avr genes matched perfectly the expected phenotype for each of the seven Avr genes, thus confirming the specificity of the primers for the targeted region (FIGS. 2, 4, 6, 8, 10, 12, 14). For six of the seven genes, a single set of primers was sufficient to discriminate the haplotypes leading to a virulent or avirulent reaction. In the case of Avr1a, four different haplotypes were exploited so three different pairs of primers were designed and included simultaneously in the molecular assay to cover the spectrum of possible haplotypes: Avr1a-indel, Avr1a-snp1 and Avr1a-snp2 (FIG. 2). As such, the combined presence of all three amplicons indicates avirulence.

Multiplex PCR Diagnostic Tool

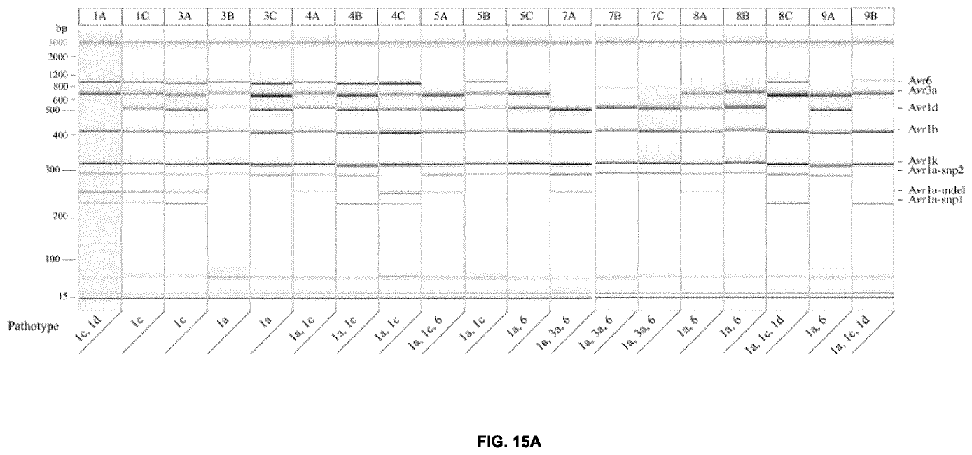

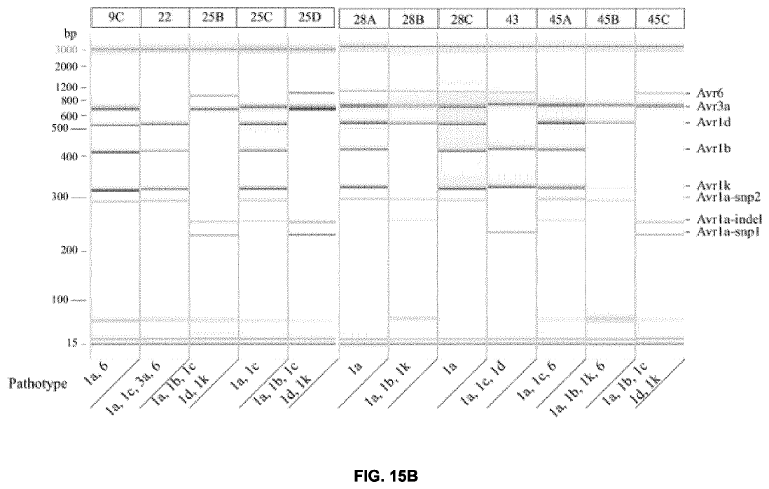

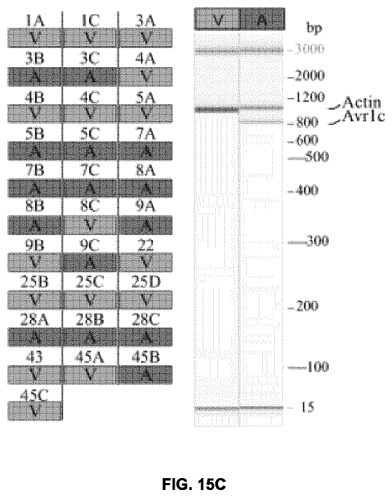

Once all primers were individually tested for their ability to discriminate between virulent and avirulent isolates for each avirulence gene, they were mixed together in a single PCR reaction to check their compatibility in a multiplex PCR diagnostic tool. Following optimization of the PCR conditions, the molecular assay was carried out in two parallel runs: one multiplex assay for the detection of Avr1a, Avr1b, Avr1d, Avr1k, Avr3a and Avr6, and one assay for Avr1c. The presence of a band of a specific size as described in Table 1 indicates that the tested isolate carries the avirulent allele associated with the amplicon of the Avr gene of that size. Conversely, the absence of an amplicon for a given gene indicates that the isolate carries the corresponding pathotype. For example, FIG. 15 presents results from the multiplex PCR assay on the 31 known isolates with their corresponding pathotype based on a phenotypic assay. Results show that the pathotype, as expressed by the absence of an amplicon for a given gene, is accurately predicted by the molecular assay. As an illustration, isolate 1A shows amplicons for Avr1a, 1b, 1k, 3a and 6 (FIG. 15A) and none for 1d and 1c (FIG. 15C), which translates into pathotype 1c and 1d.

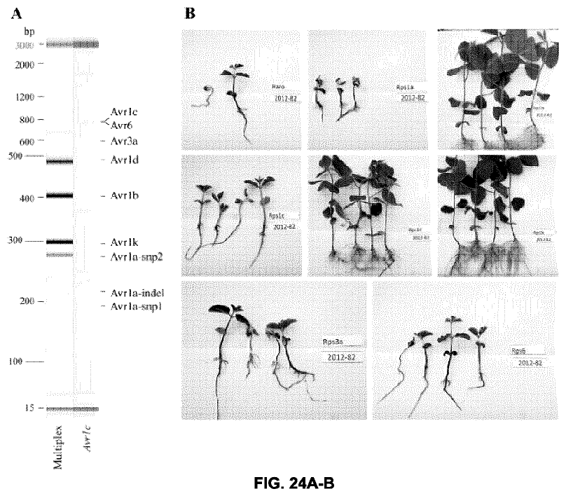

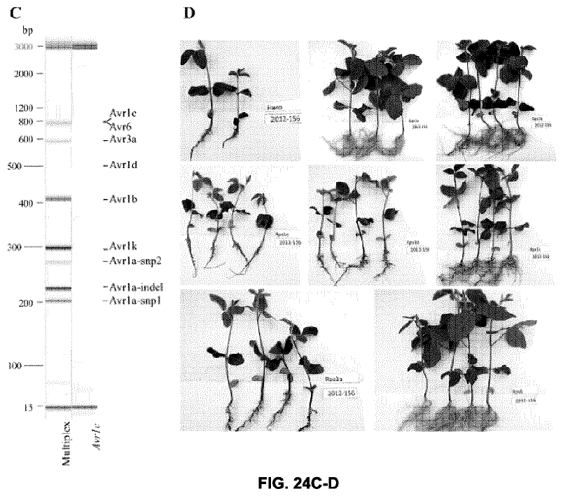

Genotyping and phenotyping of isolates with unknown pathotype After validation of the multiplex PCR assay with the 31 known isolates, 15 uncharacterized isolates were randomly selected to confirm the effectiveness of the assay. Representative results obtained following the molecular and hydroponic assays are presented for two isolates (FIG. 24). As seen in FIG. 24A, the presence of amplicons for Avr1b, Avr1d and Avr1k on the gel is indicative that isolate 2012-82 should have pathotype 1a, 1c, 3a, 6. When compared with the bioassay (FIG. 24B), the phenotypes obtained clearly corroborated the molecular assay where a compatible interaction was observed between the isolate and differentials Rps1a, 1c, 3a and 6. In the other example with isolate 2012-156 (FIG. 24C), the molecular assay showed amplification for Avr1a (Avr1a-snp1, Avr1a-indel, Avr1a-snp2), 1b, 1k, 3a and 6, which leads to a diagnostic of pathotype 1c, 1d for that isolate. Interestingly, the phenotypic assay shown in FIG. 24D confirmed the compatible interaction with differentials Rps1c and 1d but also suggested one with Rps3a, in spite of the molecular assay clearly showing an amplicon for Avr3a. As a matter of fact, when results were combined for all 15 isolates and seven Avr genes (105 interactions), there was only a single and similar discrepancy when the molecular assay and the phenotypes did not match perfectly (Table 3). Indeed, in four cases, a compatible interaction was observed with Rps3a in the hydroponic assay, in spite of the presence of an amplicon for the avirulent allele of Avr3a. All other interactions generated a perfect match between the molecular and the phenotyping assay for a prediction accuracy of 96% (101/105).

TABLE 3
Comparative results of predicted pathotypes between the molecular assay
and the hydroponic assay for 15 isolates of Phytophthora sojae

| | Predicted pathotype | Observed pathotype^{‡} |
|---|---|---|
| Isolate | (Molecular assay) | (Hydroponic assay) |
| 2010-29 | 1a, 1c | 1a, 1c, 3a |
| 2010-32 | 1a, 1c | 1a, 1c |
| 2010-42 | 1a, 1c | 1a, 1c |
| 2010-44 | 1a, 1c | 1a, 1c, 3a |
| 2011-35 | 1a, 1c, 3a | 1a, 1c, 3a |
| 2011-40 | 1a, 1b, 1c, 1k | 1a, 1b, 1c, 1k |
| 2012-01 | 1a, 1c, 3a, 6 | 1a, 1c, 3a, 6 |
| 2012-120 | 1a, 1c, 6 | 1a, 1c, 6 |
| 2012-127 | 1a, 1c | 1a, 1c |
| 2012-136 | 1a, 1c, 6 | 1a, 1c, 6 |
| 2012-156 | 1c, 1d | 1c, 1d, 3a |
| 2012-40 | 1a, 1c, 6 | 1a, 1c, 6 |
| 2012-57 | 1a, 1c, 1d | 1a, 1c, 1d |
| 2012-76 | 1a, 1c | 1a, 1c, 3a |
| 2012-82 | 1a, 1c, 3a, 6 | 1a, 1c, 3a, 6 |

^{‡}Underlined pathotype indicates discrepancy with the molecular assay

The studies described herein present the first molecular assay aimed at identifying Avr genes for the purpose of diagnosing the virulence profile of a plant pathogen. Up to this point, the only way to determine the pathotype of a given isolate was through cumbersome and long phenotyping procedures, each with its own shortcomings (Dorrance et al., 2008, Lebreton et al., 2018). Through this unique molecular assay, based on discriminant haplotypes at seven Avr genes of P. sojae, it should now be possible to obtain a rapid and accurate identification of the virulence profile of isolates in order to precisely select soybean material carrying the appropriate Rps genes.

Since the deployment of the first Rps gene in soybean, Rps1a, P. sojae has demonstrated a very strong resilience and ability to adapt to selection pressure through rapid mutations of Avr genes (Drenth et al., 1996, Kaitany et al., 2001, Keeling, 1984, Layton et al., 1986). As a result, the pathogen has evolved a staggering pathotype diversity (Dorrance, 2018, Dorrance et al., 2016, Dorrance et al., 2003, Sugimoto et al., 2012) that threatens current efforts to control its spread through genetic approaches. For instance, in a survey of P. sojae isolate diversity in Canada, Xue et al. (2015) reported an important shift in virulence over time, whereas most isolates now overcome Rps1k, the most recently introduced Rps gene, while this pathotype was completely absent in Canadian fields 20 years ago. This ability to alter Avr genes appears to be based on mutations that range from complete deletion of the gene or copy number variation, to presence of indels or single point mutations within or in close proximity to the gene (Qutob et al., 2009, Tyler and Gijzen, 2014).

Provided herein is an exhaustive description and comparison of haplotype diversity at seven Avr genes (1a, 1b, 1c, 1d, 1k, 3a, 6) of P. sojae through whole-genome sequencing of 31 isolates with different pathotypes. Their results offer a precise blueprint of sequence variation and conservation in each gene, results that we exploited to identify discriminant regions associated with different phenotypes.

In the process of trying to develop a molecular assay based on the discriminant haplotypes, several challenges were encountered at different stages of the study. First, acquisition of virulence for a pathogen is often due to a partial or complete deletion of the Avr gene. In the studies described herein, this phenomenon was only systematically observed in the case of Avr1d, which meant that we had to conduct an exhaustive analysis of the upstream and downstream regions of the other Avr genes to find SNPs/indels that would segregate haplotypes associated with avirulence from those associated with virulence. In certain instances, as for Avr1a, 1c and 6, the discriminating regions were located outside of the coding region of the gene.

When the molecular assay was applied to the 31 isolates that we had also phenotyped, we were able to show a perfect adequation between the two approaches. This confirms that the molecular assay constitutes a valid substitute to the long phenotyping assays and offers a much more practical approach to determine pathotypes of P. sojae isolates. It can thus find applications in delineating with precision the deployment of soybean lines carrying the proper Rps genes to overcome the pathotypes present in a given environment. Furthermore, as new resistance genes are discovered and introgressed into soybean, the test can be adapted to include new Avr genes and follow the evolution of new pathotypes over time. Finally, it may be used to assess other gene-for-gene dependent plant-pathogen interactions.

Upon testing the accuracy of the molecular assay with 15 isolates of unknown virulence, we notably observed a significant level of validation of the molecular assay, i.e. a 96% level of concordance between the molecular and the phenotyping assays (the only four cases of discrepancy out of 105 interactions tested were related to Avr3a). In summary, described herein is a comprehensive molecular assay capable of defining the pathotypes of Phytophthora pathogens, based on Avr genes, with unprecedented ease and precision. Its other advantages can be found in eliminating the shortcomings of the different phenotyping procedures while reducing time and resources involved in the process. It can also have practical applications for breeders and growers in management of the disease with a tailored deployment of use of Rps genes based on a precise and rapid determination of pathotypes present in a given area.

TABLE 4
SNPs and indels described herein

| Gene | SNP(s) | Indel(s) | FIGS. |
|---|---|---|---|
| Avr1a | 2033340 (A to T) 2046815 (A to G) | Deletion within positions 2042427 and 2042445 | 1, 16, 23 |
| | 2067663 (T to C) | | |
| | 2214437 (C to T) | | |
| | 2263686 (C to G) | | |
| | 2263687 (A to G) | | |
| | 2263686-2263687 (CA to GG) | | |
| | 1799779 (G to A) | | |
| | 1799780 (A to C) | | |
| | 1799779-1799780 (GA to AC) | | |
| Avr1b | 3146564 (T to G) | 3146481: Indel (TAAG vs. T), deletion of AAG at | 3, 17, 23 |
| | 3146466 (G to A) | positions 3146482 to 3146484; | |
| | 3146467 (G to A) | 3146477: Indel (T vs. TGCA), insertion of GCA | |
| | 3146468 (G to A) | between positions 3146477 and 3146478 | |
| | 3146466-3146467 (GG to AA) | | |
| | 3146467-3146468 (GG to AA) | | |
| | 3146466-3146468 (GGG to AAA) | | |
| Avr1c | 2046577 (C to T) | Deletion | 5, 18, 23 |
| | 2046694 (C to A) | | |
| | 2046815 (A to G) | | |
| | 2046839 (T to A) | | |
| | 2046037 (G to C) | | |
| | 2046038 (A to C) | | |
| | 2046037-2046038 (GA to CC) | | |
| | 2046815 (G to A) | | |
| | 2046817 (A to T) | | |
| | 2046819 (G to A) | | |
| | 2046821 (C to A) | | |
| Avr1d | 5920122 (A to G) | Deletion within positions 5919167 and 5922387 | 7, 19, 23 |
| Avr1k | 3142750 (T to G) | 3143184: Indel (T vs. TAAGTAGCA); insertion of | 9, 20, 23 |
| | 3142827 (G to A) | AAGTAGCA between positions 3143284 and | |
| | 3142512 (T to C) | 3143285 | |
| | 3142519 (C to G) | | |
| Avr3a | 615438 (T to A) | 615329: Indel (CAAAGAT vs. C), deletion of | 11, 21, 23 |
| | | AAAGAT at positions 615330-615335 | |
| Avr6 | | 7223782: Indel (AGAGCCTCAAGCTTG [SEQ ID | 13, 22, 23 |
| | | NO: 89] vs. A); deletion of GAGCCTCAAGCTTG | |
| | | (SEQ ID NO: 90) at positions 7223783-7223796) | |

TABLE 5
Correspondence of SNP/Indel position(s) with positions in SEQ NOs: 1-70

| Gene | SNP(s)/Indel(s) | Corresponding position(s) in SEQ ID NOs: 1-70 | FIG. |
|---|---|---|---|
| Avr1a | 2033340 (A to T) | Position 1001 in SEQ ID NOs: 1-6 | 16C |
| | 2046815 (A to G) | Position 693 in SEQ ID NOs: 13-19 | 16L |
| | 2067663 (T to C) | Position 1001 in SEQ ID NOs: 20-25 | 16Q |
| | 2214437 (C to T) | Position 1001 in SEQ ID NOs: 26-28 and 31; Position 1010 | 16V |
| | | in SEQ ID NOs: 29-30 | |
| | 2263686 (C to G) | Position 1016 in SEQ ID NOs: 32-38 | 16AA |
| | 2263687 (A to G) | Position 1017 in SEQ ID NOs: 32-38 | 16AA |
| | 2263686-2263687 (CA to | Positions 1016-1017 in SEQ ID NOs: 32-38 | 16AA |
| | GG) | | |
| | 1799779 (G to A) | Position 1261 in SEQ ID NOs: 39-44 | 16GG |
| | 1799780 (A to C) | Position 1262 in SEQ ID NOs: 39-44 | 16GG |
| | 1799779-1799780 (GA to | Positions 1261-62 in SEQ ID NOs: 39-44 | 16GG |
| | AC) | | |
| | Deletion within positions | 2042427 corresponds to position 1229 in SEQ ID NOs: | 16I |
| | 2042427 and 2042445 | 13-18; 2042445 corresponds to position 1247 in SEQ ID NOs: | |
| | | 13-18 | |
| Avr1b | 3146564 (T to G) | Position 1401 in SEQ ID NOs: 45-46; Position 1404 in SEQ | 17D |
| | | ID NO: 47; Position 1412 in SEQ ID NOs: 48-49 | |
| | 3146466 (G to A) | Position 1306 in SEQ ID NOs: 45-46; Position 1309 in SEQ | 17D |
| | | ID NO: 47; Position 1317 in SEQ ID NOs: 48-49 | |
| | 3146467 (G to A) | Position 1307 in SEQ ID NOs: 45-46; Position 1310 in SEQ | 17D |
| | | ID NO: 47; Position 1318 in SEQ ID NOs: 48-49 | |
| | 3146468 (G to A) | Position 1308 in SEQ ID NOs: 45-46; Position 1311 in SEQ | 17D |
| | | ID NO: 47; Position 1319 in SEQ ID NOs: 48-49 | |
| | 3146466-3146467 (GG to | Positions 1306-1307 in SEQ ID NOs: 45-46; Positions | 17D |
| | AA) | 1309-1310 in SEQ ID NO: 47; Positions 1317-1318 in SEQ ID | |
| | | NOs: 48-49 | |
| | 3146467-3146468 (GG to | Positions 1307-1308 in SEQ ID NOs: 45-46; Positions | 17D |
| | AA) | 1310-1311 in SEQ ID NO: 47; Positions 1318-1319 in SEQ ID | |
| | | NOs: 48-49 | |
| | 3146466-3146468 (GGG to | Positions 1306-1308 in SEQ ID NOs: 45-46; Positions | 17D |
| | AAA) | 1309-1311 in SEQ ID NO: 47; Positions 1317-1319 in SEQ ID | |
| | | NOs: 48-49 | |
| | 3146481: Indel (TAAG vs. | 3146481 corresponds to position 1318 in SEQ ID NOs: | 17D |
| | T), deletion of AAG at | 45-46, position 1321 in SEQ ID NO: 47, position 1332 in SEQ | |
| | | ID NO: 48, position 1329 in SEQ ID NO: 49; 3146482 to | |
| | positions 3146482 to | 3146484 correspond to positions 1319-1321 in SEQ ID | |
| | 3146484; | NOs: 45-46, position 1322-1324 in SEQ ID NO: 47, position | |
| | | 1330-1332 in SEQ ID NO: 49 | |
| | 3146477: Indel (T vs. | 3146477 corresponds to position 1317 in SEQ ID NOs: | 17D |
| | TGCA), insertion of GCA | 45-46, position 1320 in SEQ ID NO: 47, position 1328 in SEQ | |
| | between positions 3146477 | ID NOs: 48-49 | |
| | and 3146478 | | |
| Avr1c | 2046577 (C to T) | Position 1557 in SEQ ID NOs: 50-53 and 56; Position 1560 | 18D |
| | | in SEQ ID NO: 54 | |
| | 2046694 (C to A) | Position 1673 in SEQ ID NOs: 50-53 and 56; Position 1676 | 18E |
| | | in SEQ ID NO: 54 | |
| | 2046815 (A to G) | Position 1795 in SEQ ID NOs: 50-53 and 56; Position 1798 | 18E |
| | | in SEQ ID NO: 54 | |
| | 2046839 (T to A) | Position 1819 in SEQ ID NOs: 50-53 and 56; Position 1822 | 18E |
| | | in SEQ ID NO: 54; position 3 in SEQ ID NO: 55 | |
| | 2046037 (G to C) | Position 1017 in SEQ ID NOs: 50-53 and 56; Position 1020 | 18C |
| | | in SEQ ID NO: 54 | |
| | 2046038 (A to C) | Position 1018 in SEQ ID NOs: 50-53 and 56; position 1021 | 18C |
| | | in SEQ ID NO: 54 | |
| | 2046037-2046038 (GA to | Positions 1017-1018 in SEQ ID NOs: 50-53 and 56; | 18C |
| | CC) | Positions 1020-1021 in SEQ ID NO: 54 | |
| | 2046817 (A to T) | Position 1797 in SEQ ID NOs: 50-53 and 56; position 1800 | 18E |
| | | in SEQ ID NO: 54 | |
| | 2046819 (G to A) | Position 1799 in SEQ ID NOs: 50-53 and 56; position 1802 | 18E |
| | | in SEQ ID NO: 54 | |
| | 2046821 (C to A) | Position 1801 in SEQ ID NOs: 50-53 and 56; position 1804 | 18E |
| | | in SEQ ID NO: 54 | |
| | Deletion | | |
| Avr1d | 5920122 (A to G) | Position 1738 in SEQ ID NOs: 57-58; position 1762 in SEQ | |
| | | ID NO: 59 | |
| | Deletion within positions | 5919167 corresponds to position 784 in SEQ ID NOs: | 19C |
| | 5919167 and 5922387 | 57-58, position 807 in SEQ ID NO: 59 | |
| | (following position | | |
| | 5919167) | | |
| Avr1k | 3142750 (T to G) | Position 1510 in SEQ ID NOs: 61-62 and 64; position 1511 | 20D |
| | | in SEQ ID NO: 63 | |
| | 3142827 (G to A) | Position 1587 in SEQ ID NOs: 61-62 and 64; position 1588 | 20D |
| | | in SEQ ID NO: 63 | |
| | 3142512 (T to C) | Position 1272 in SEQ ID NOs: 61-62 and 64; position 1273 | 20C |
| | | in SEQ ID NO: 63 | |
| | 3142519 (C to G) | Position 1279 in SEQ ID NOs: 61-62 and 64; position 1280 | 20C |
| | | in SEQ ID NO: 63 | |
| | 3143184: Indel (T vs. | Position 3143184 corresponds to position 944 in SEQ ID | 20D |
| | TAAGTAGCA); insertion of | NOs: 61-62 and 64 and position 1945 in SEQ ID NO: 63; | |
| | AAGTAGCA between | position 3143185 corresponds to position 945 in SEQ ID | |
| | positions 3143184 and | NOs: 61-62, position 1954 in SEQ ID NO: 63 and position | |
| | 3143185 | 1953 in SEQ ID NO: 64 | |
| Avr3a | 615438 (T to A) | Position 1334 in SEQ ID NO: 65-66, position 1375 in SEQ | 21C |
| | | ID NO: 67 | |
| | 615329: Indel (CAAAGAT | 615329 corresponds to position 1225 in SEQ ID NOs. 65-66 | 21C |
| | vs. C), deletion of AAAGAT | position 1272 in SEQ ID NO: 67; 615330 corresponds to | |
| | at positions 615330-615335 | position 1226 in SEQ ID NOs. 65-66; 615335 corresponds | |
| | | to position 1231 in SEQ ID NOs. 65-66 | |
| Avr 6 | 7223782: Indel | 7223782 corresponds to position 1712 in SEQ ID NOs: | 22D |
| | (AGAGCCTCAAGCTTG | 68-69, position 1711 in SEQ ID NO: 70; 7223783 corresponds | |
| | [SEQ ID NO: 89] vs. A); | to position 1713 in SEQ ID NOs: 68-69; position 7223796 | |
| | deletion of | corresponds to position 1726 in SEQ ID NOs: 68-69 | |
| | GAGCCTCAAGCTTG | | |
| | (SEQ ID NO: 90) at | | |
| | positions 7223783-7223796) | | |

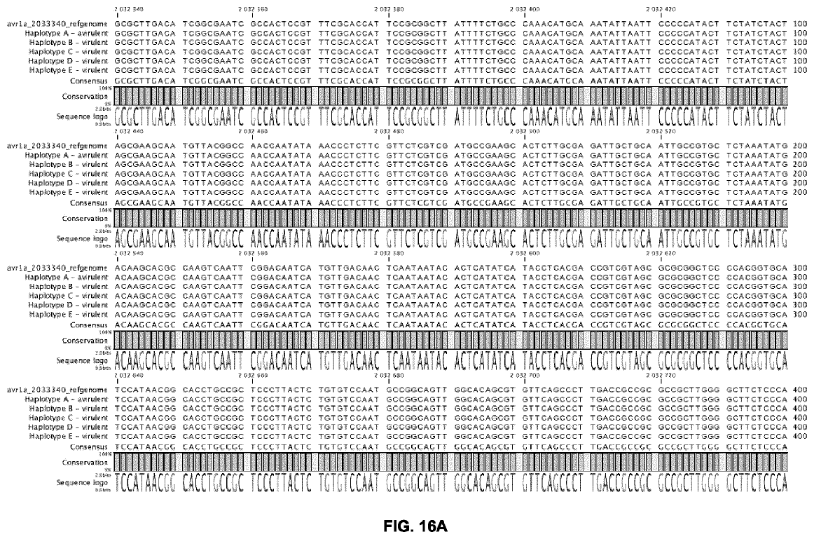

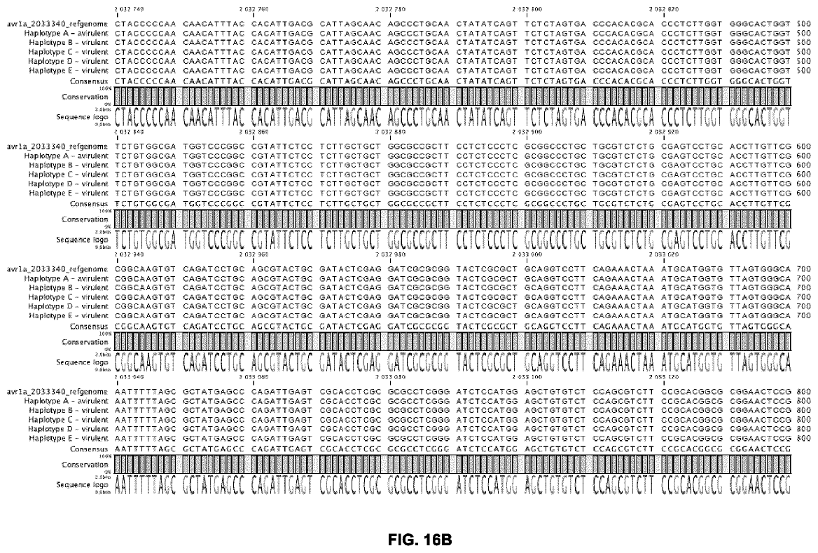

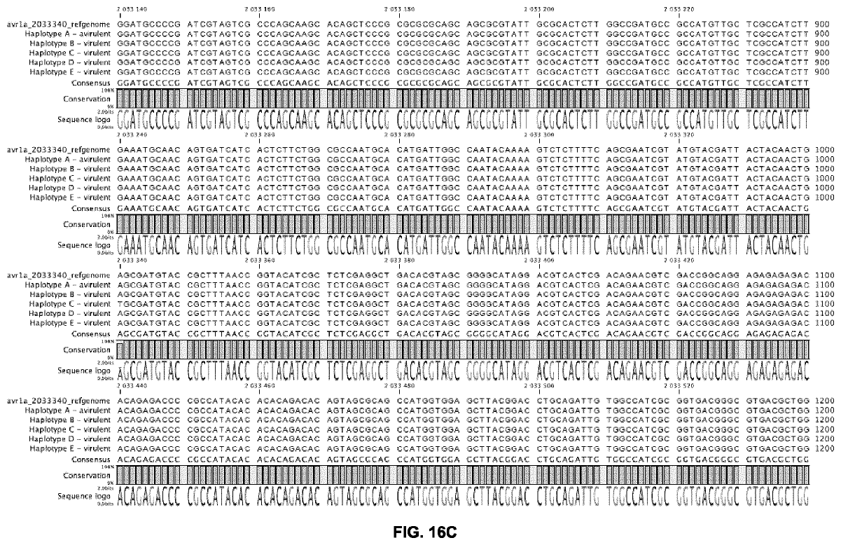

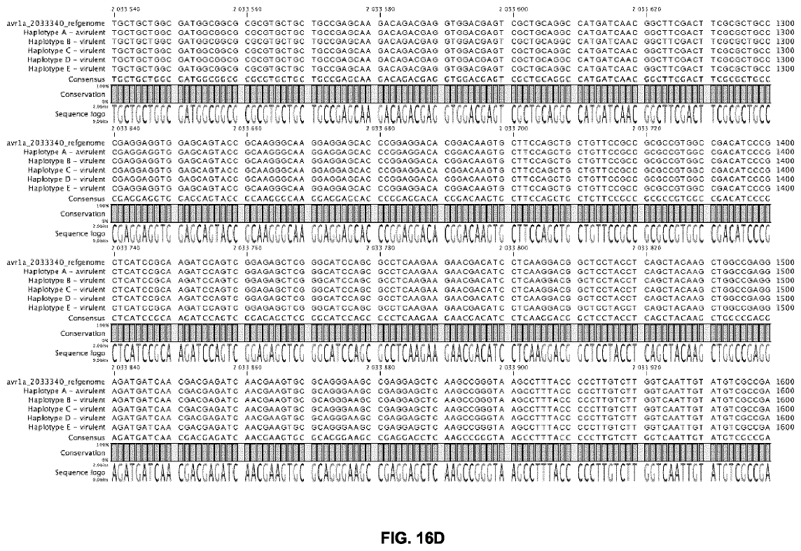

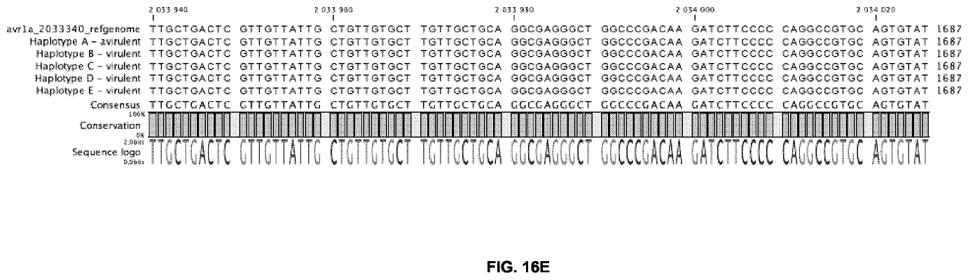

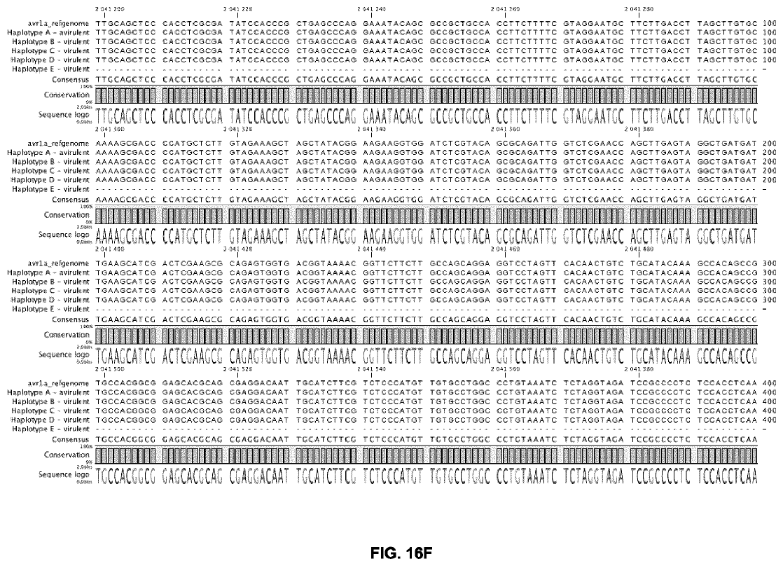

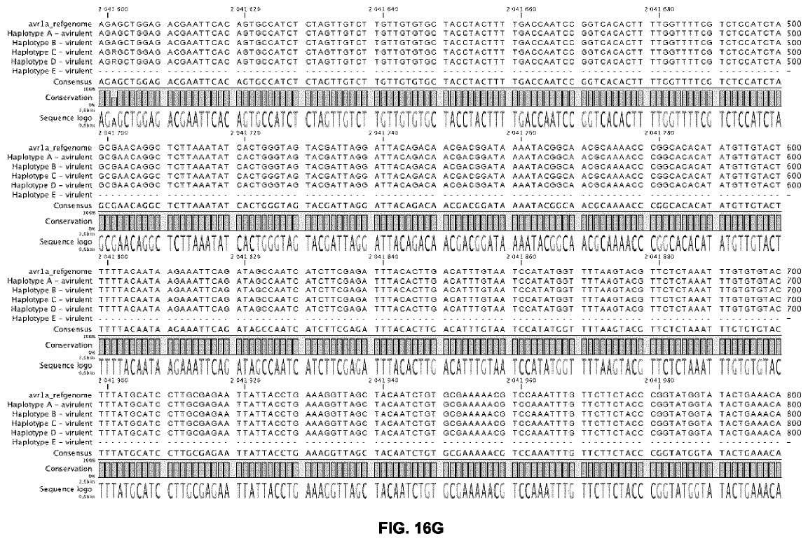

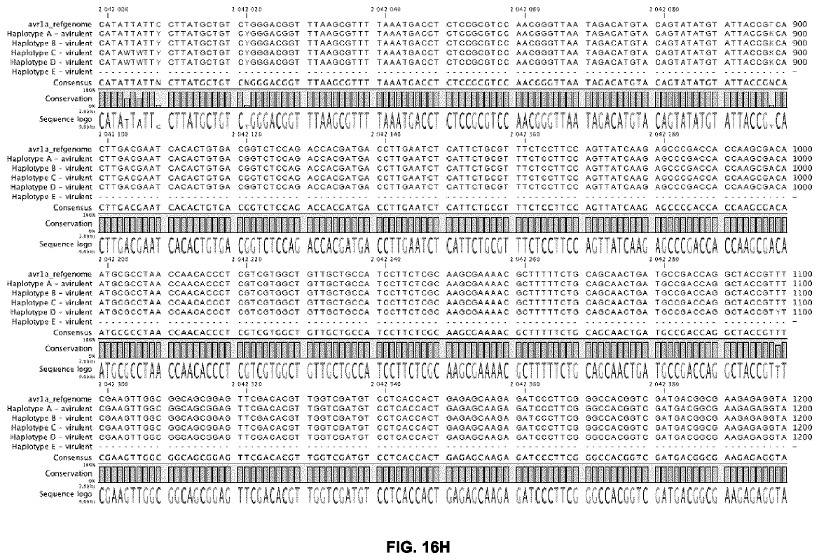

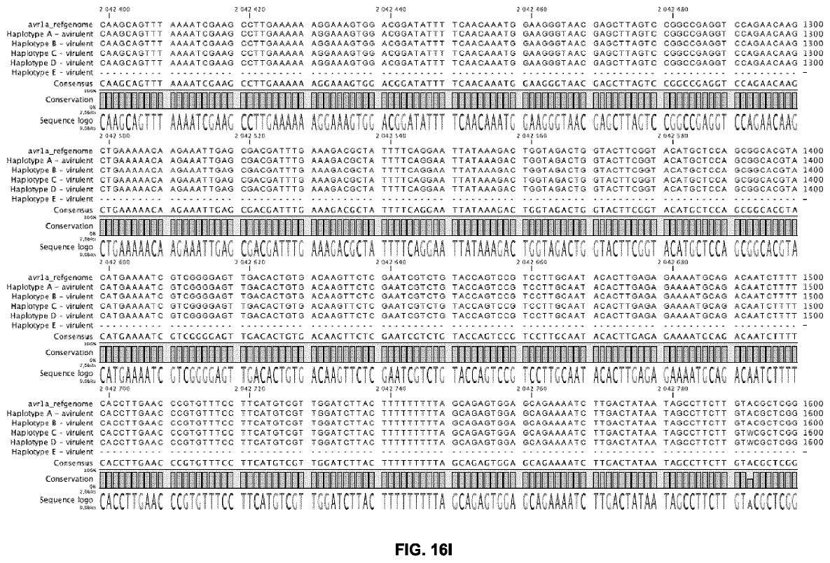

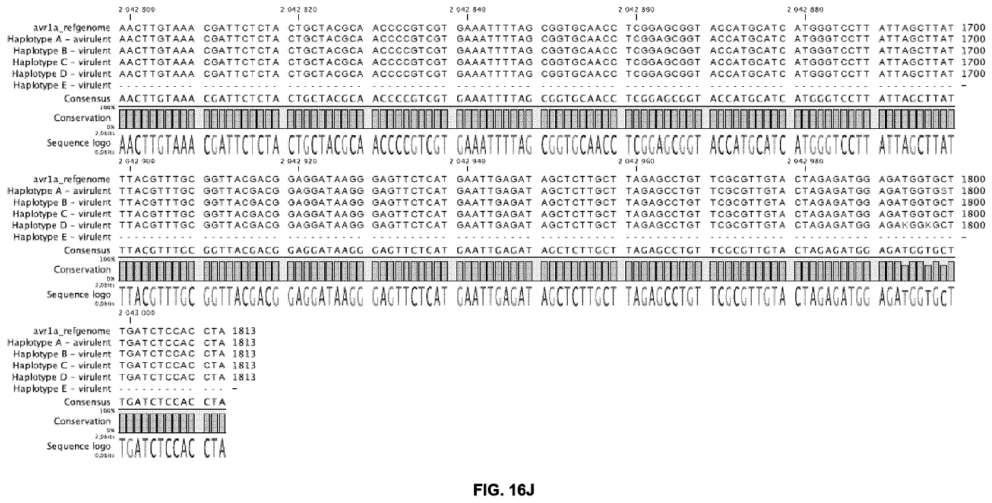

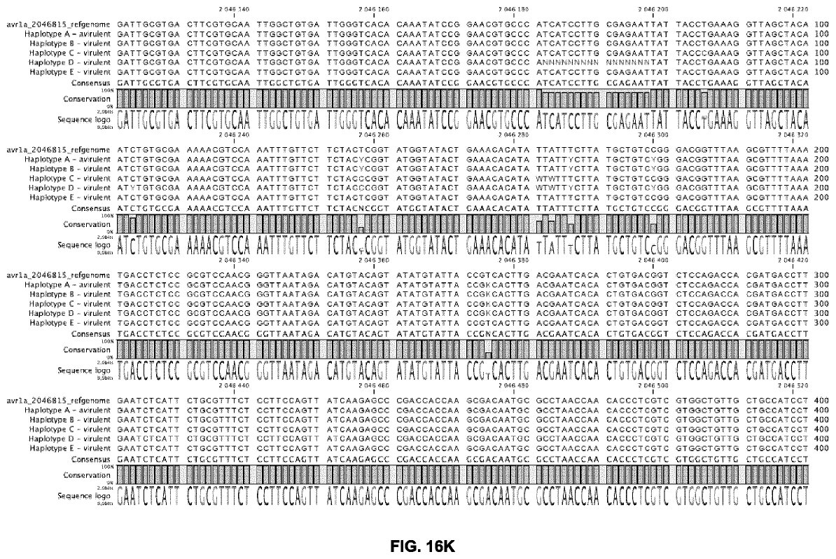

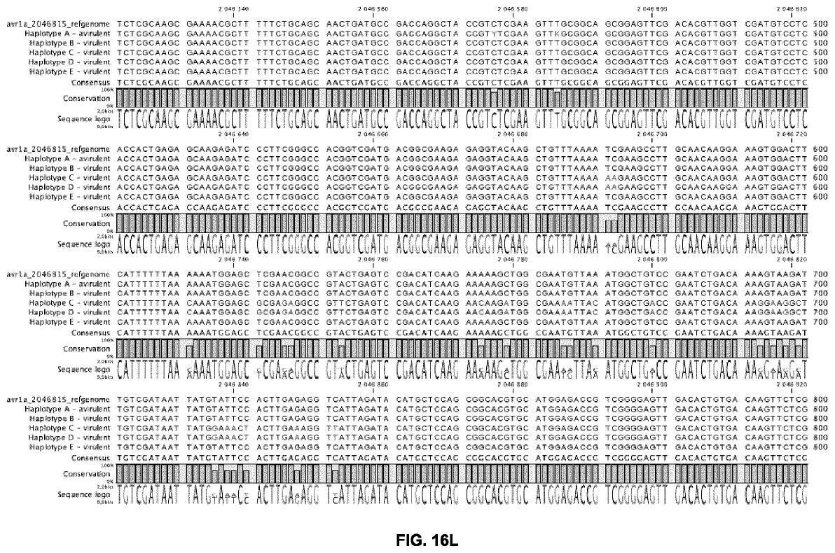

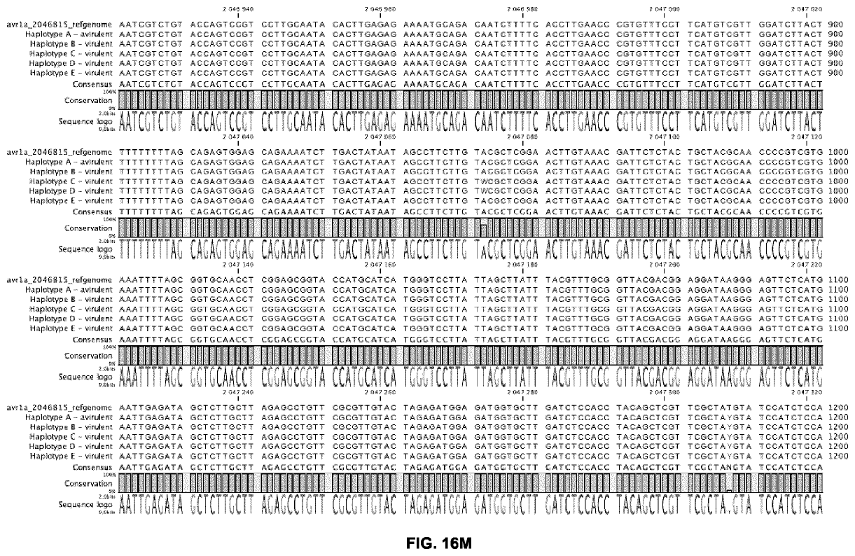

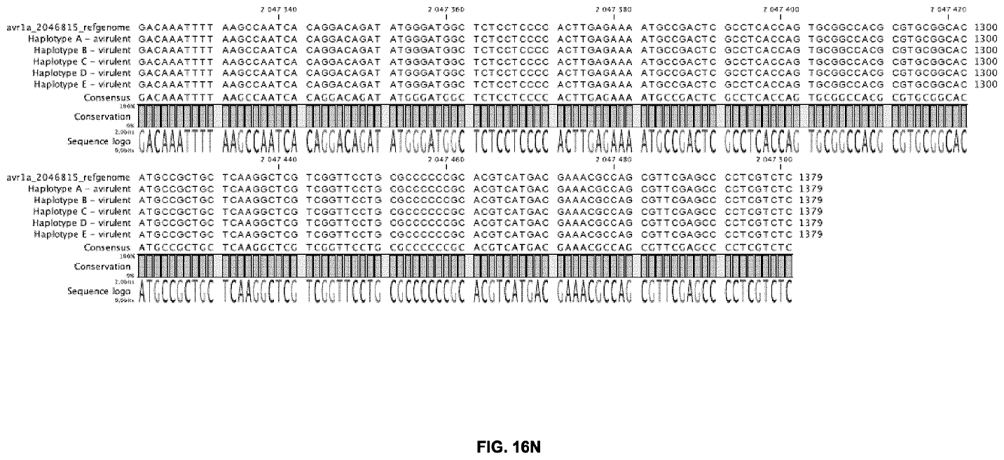

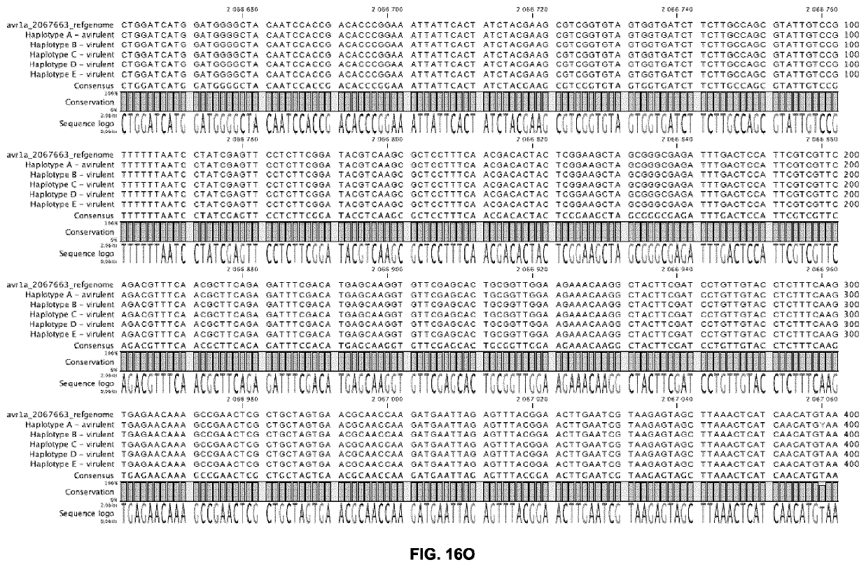

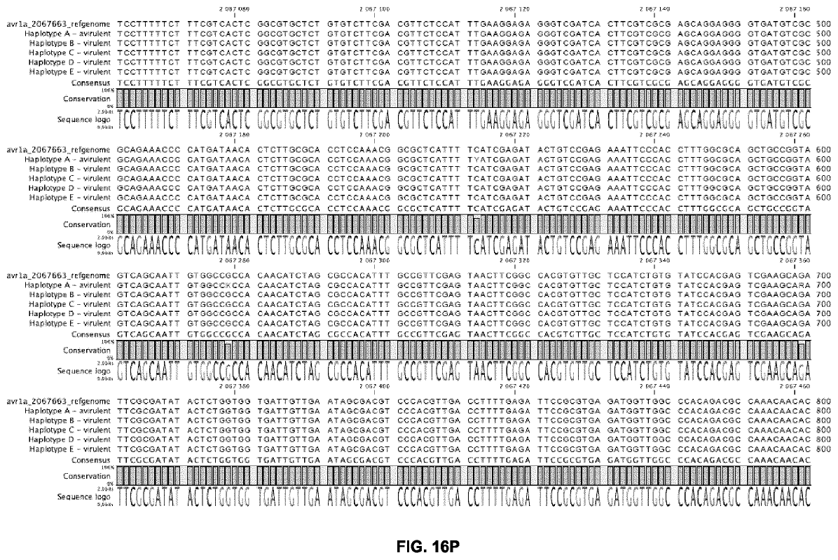

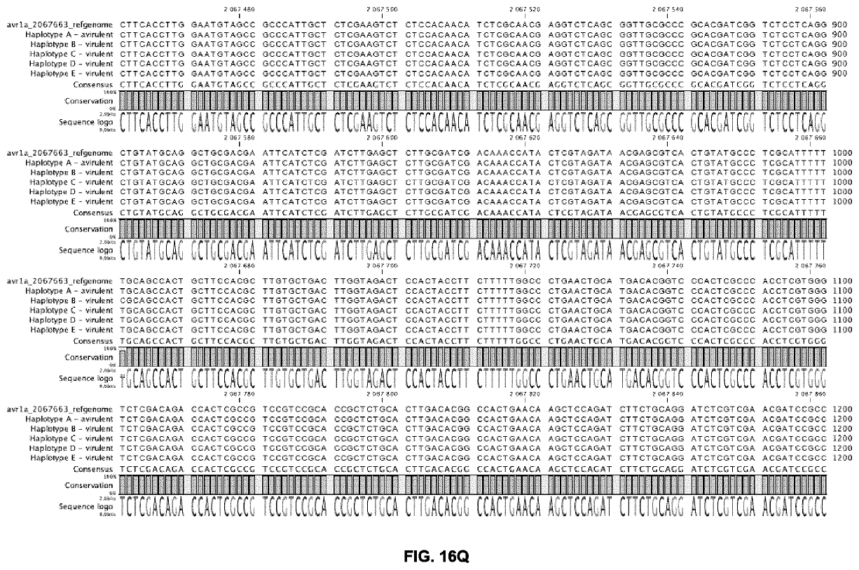

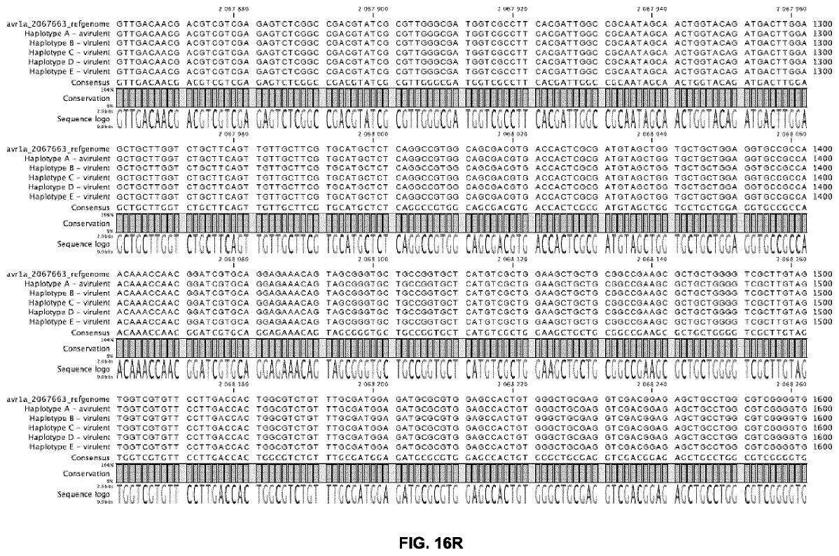

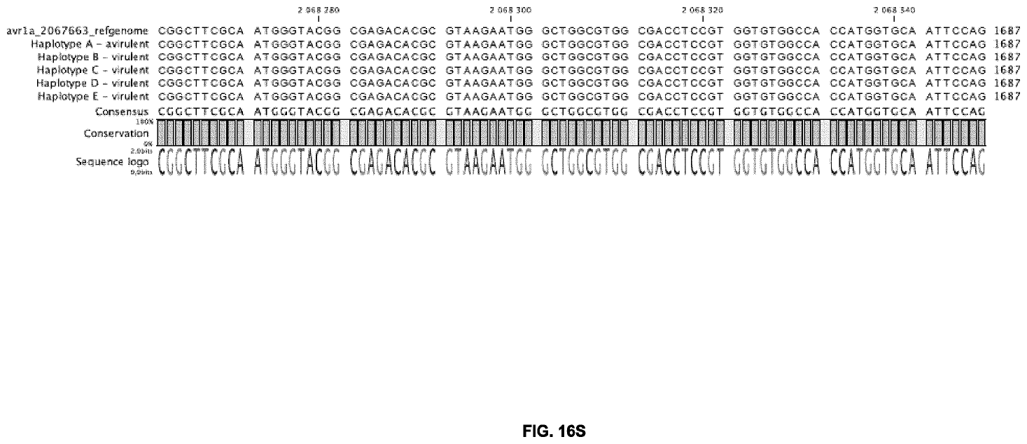

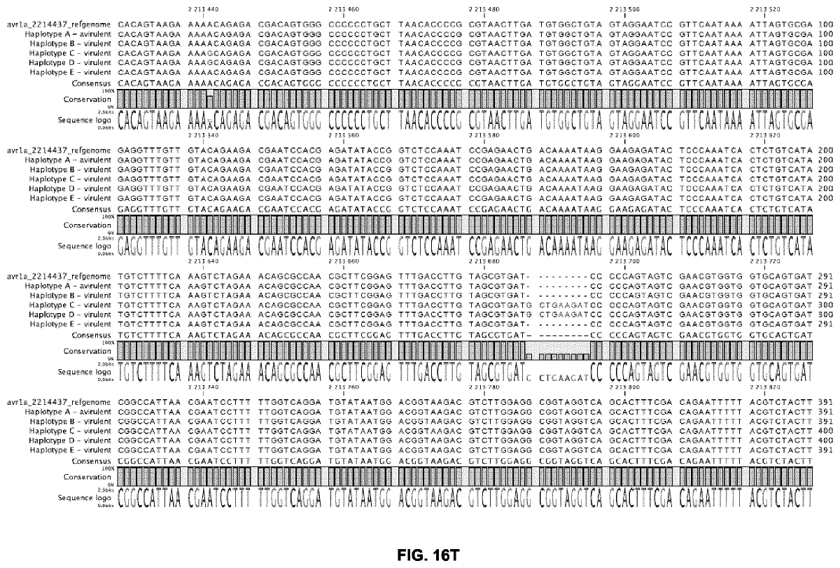

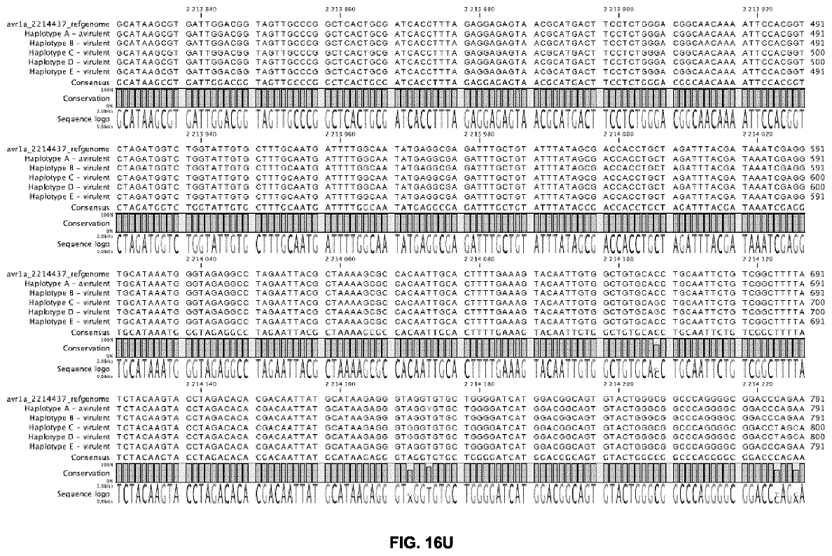

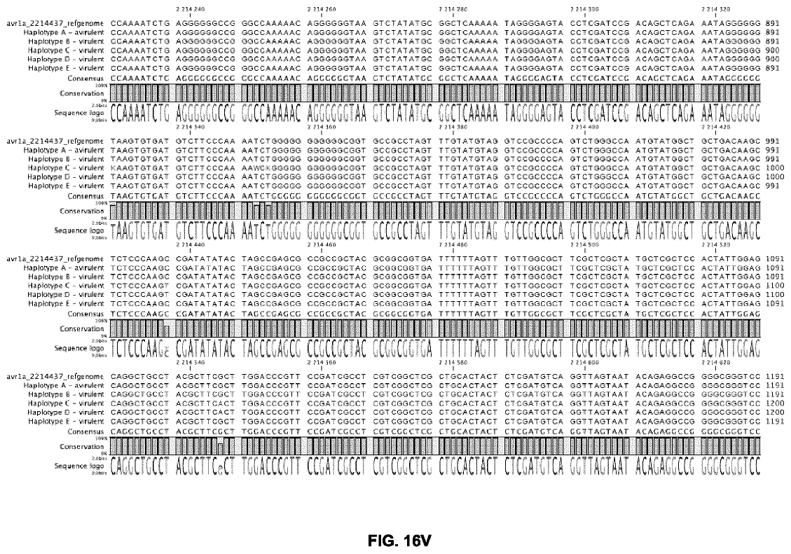

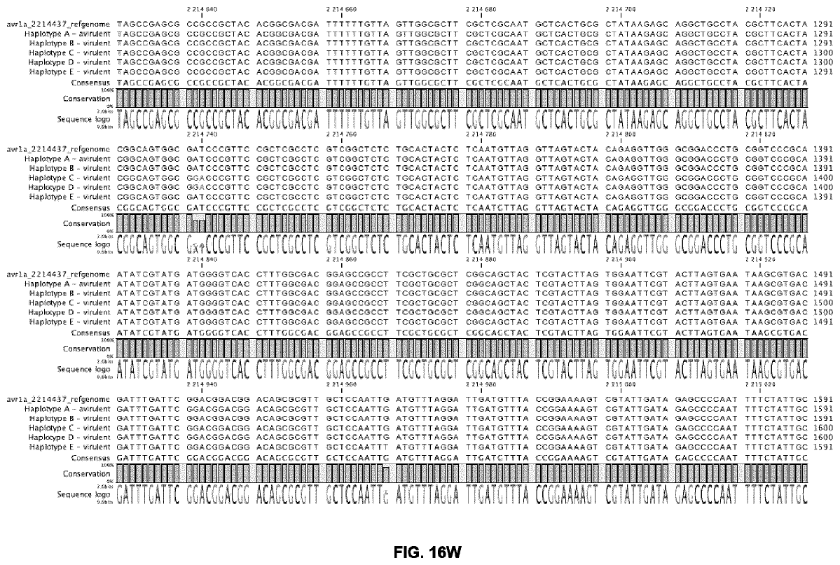

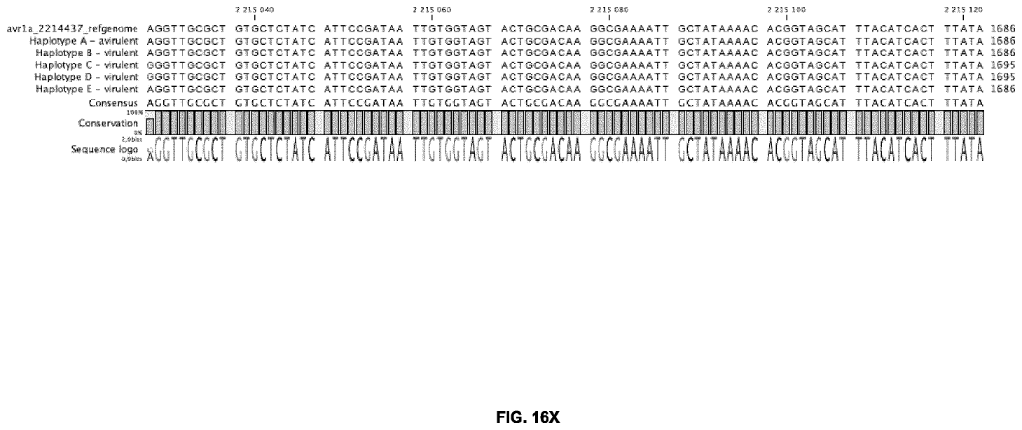

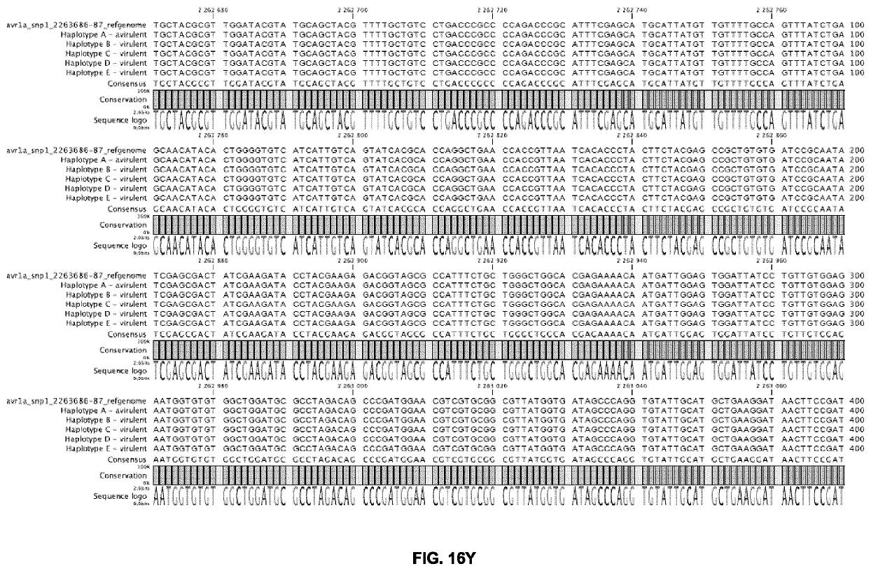

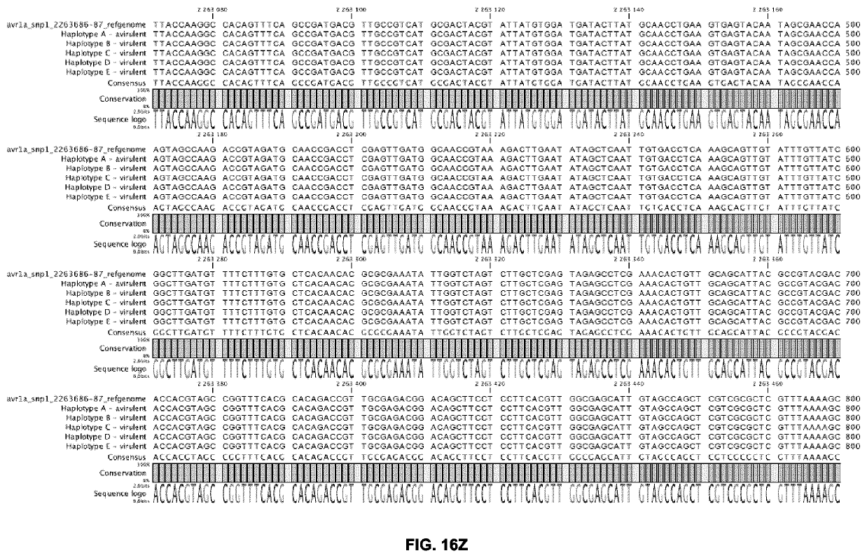

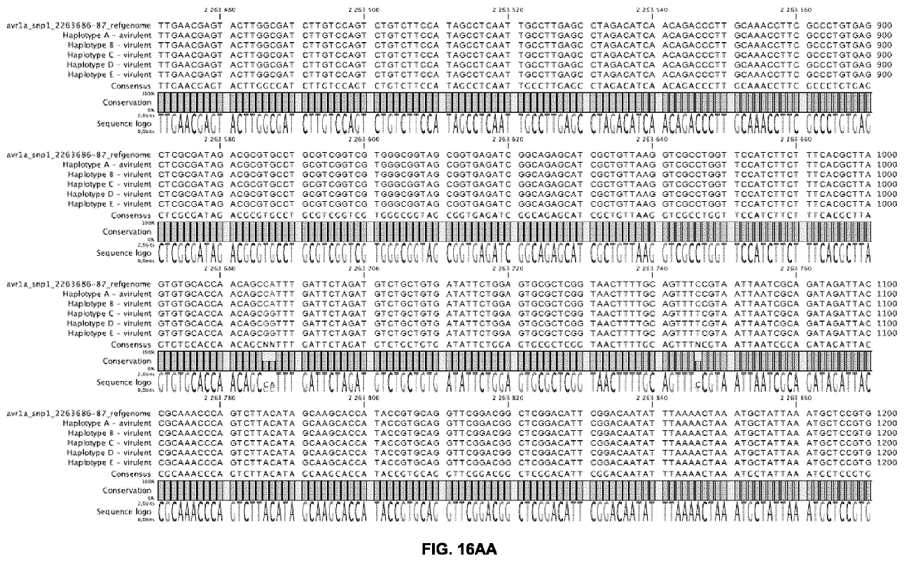

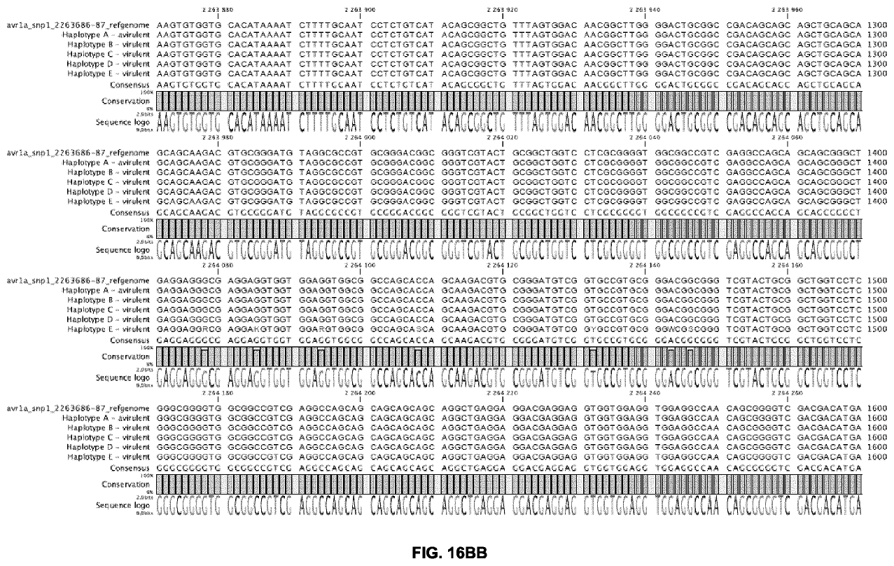

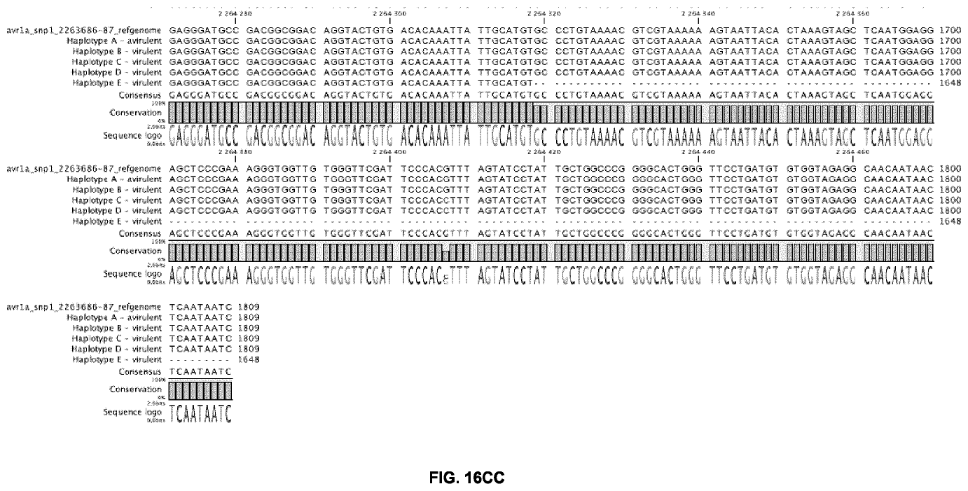

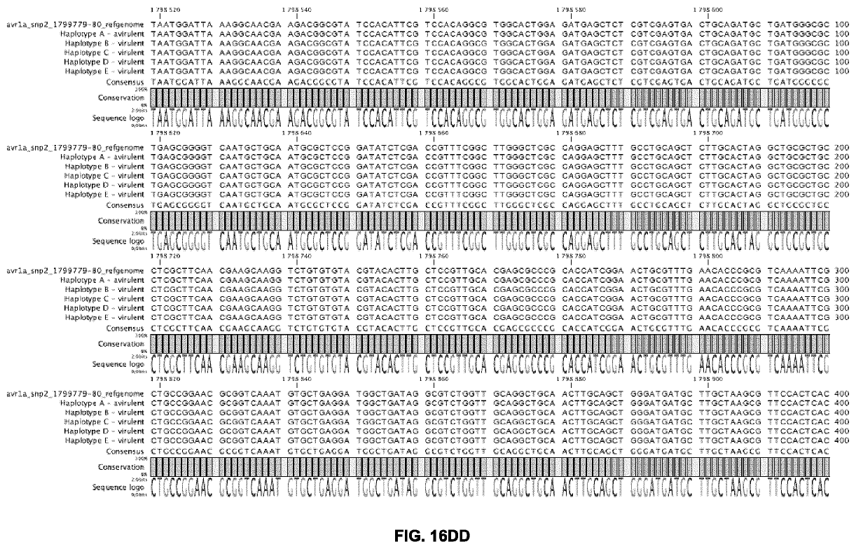

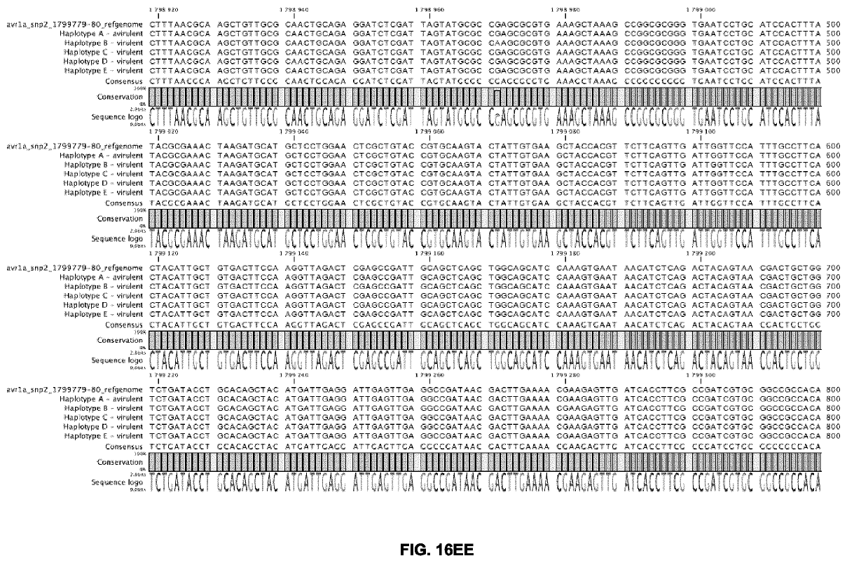

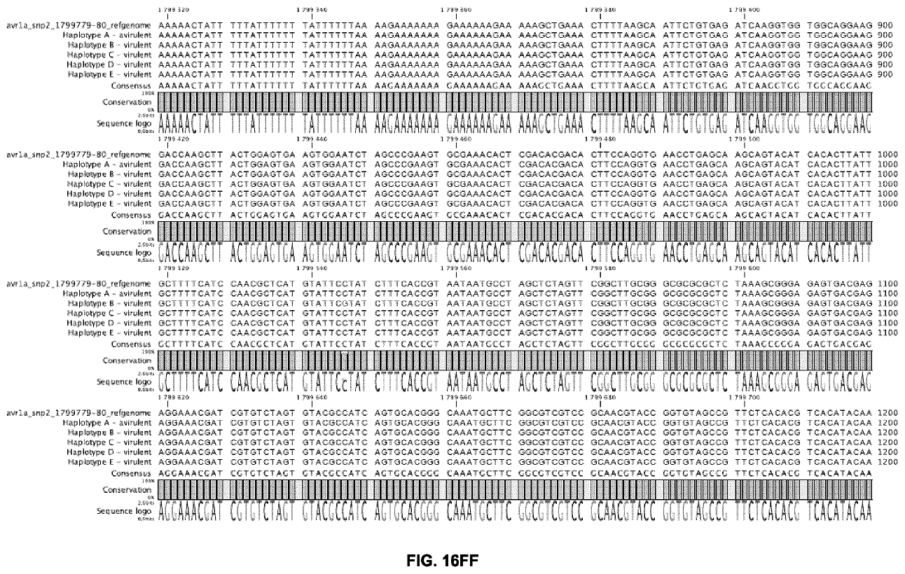

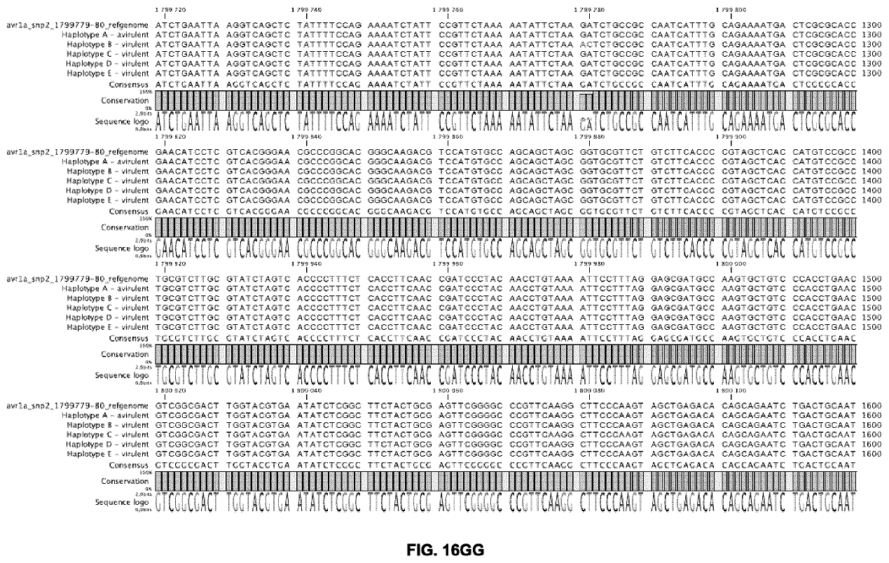

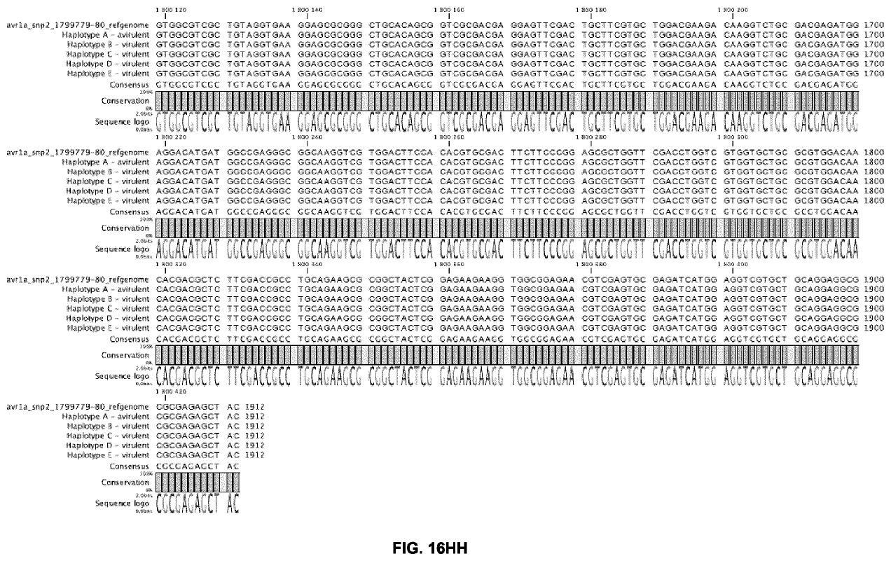

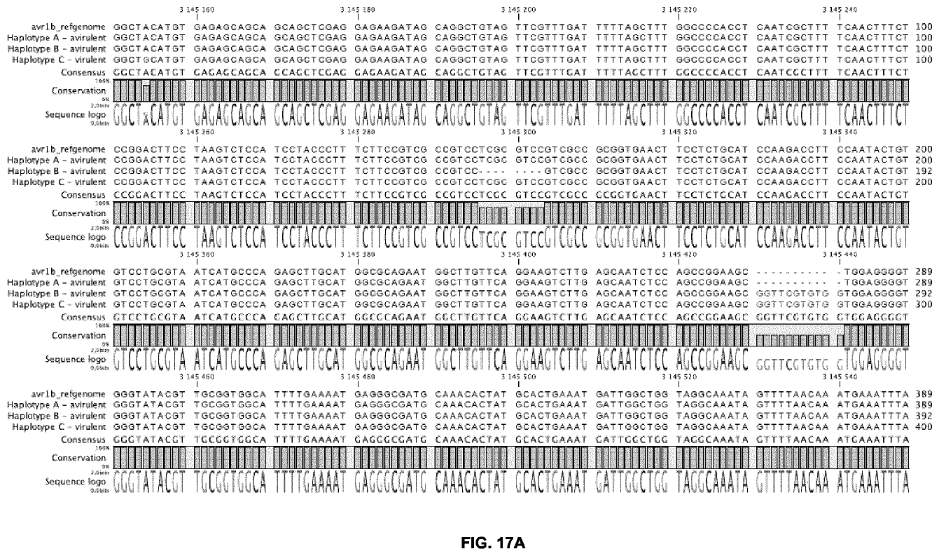

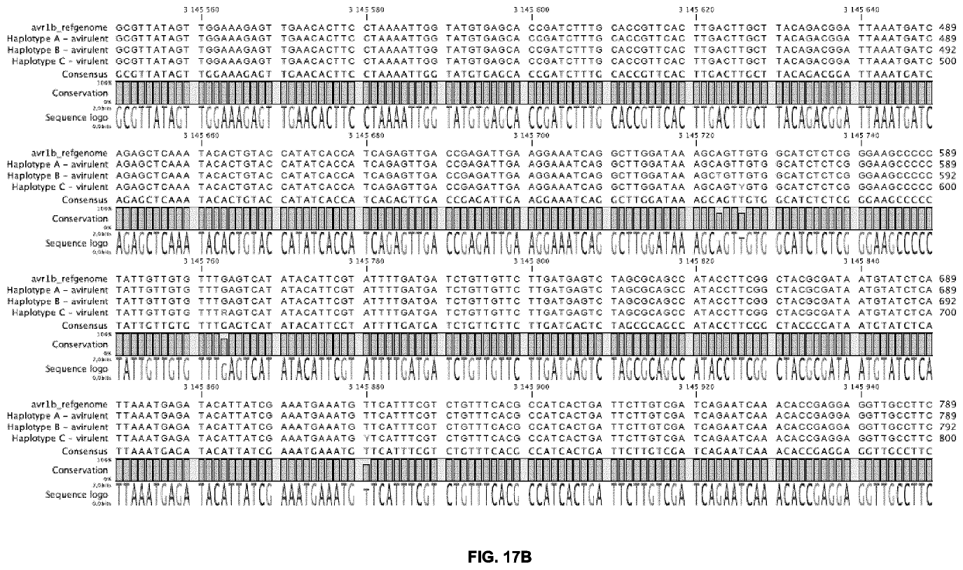

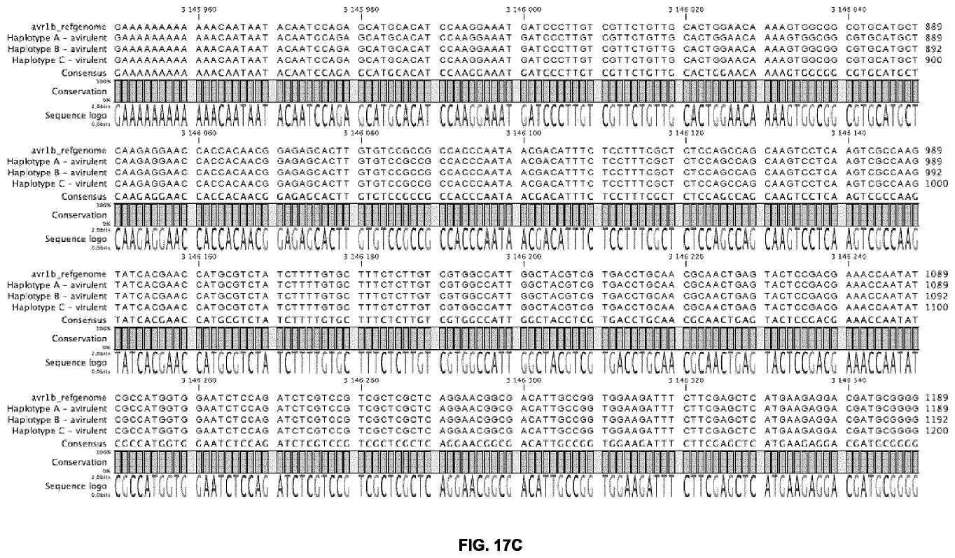

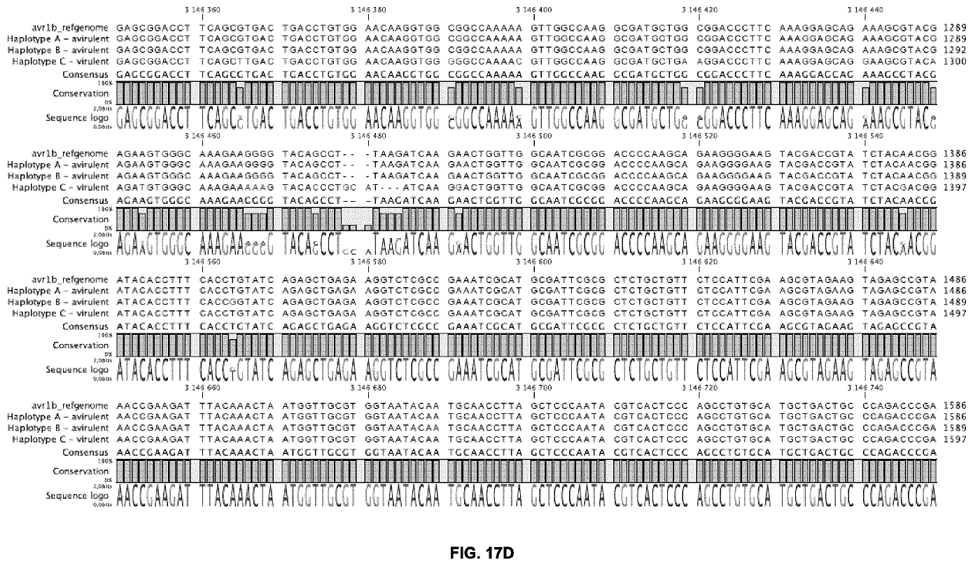

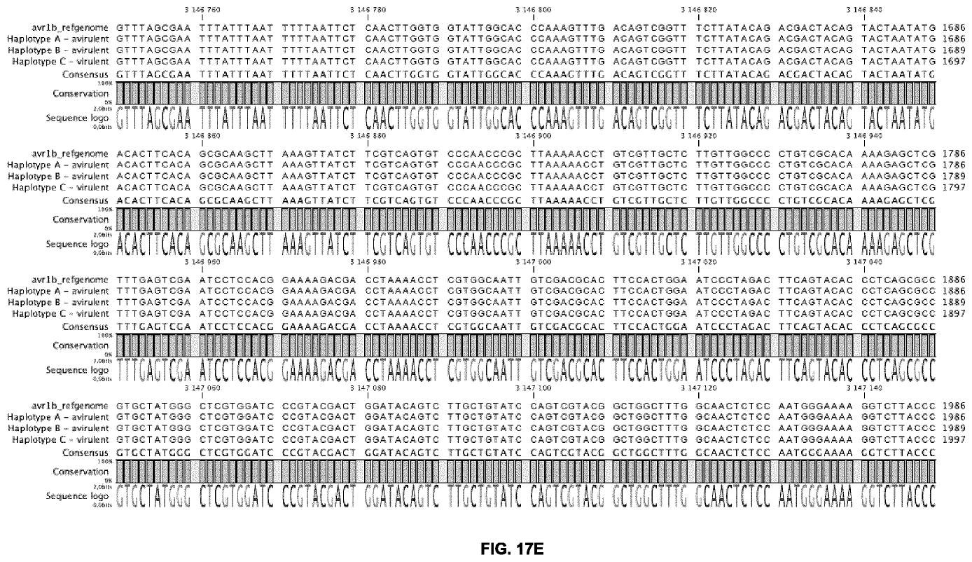

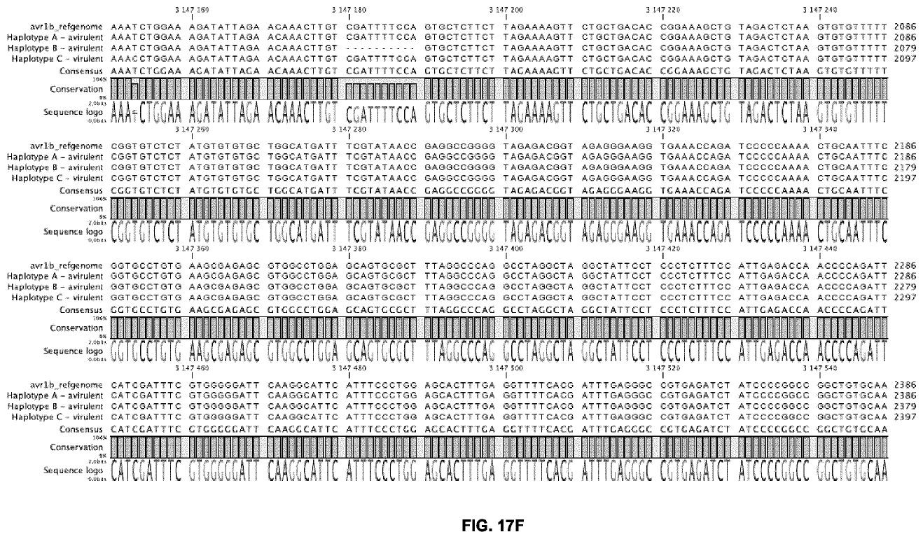

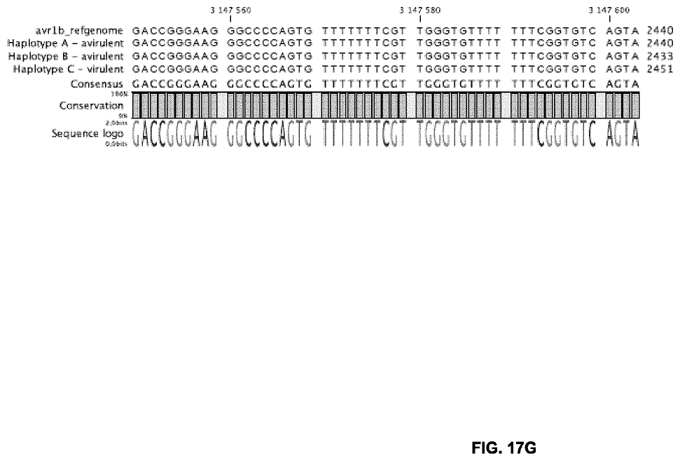

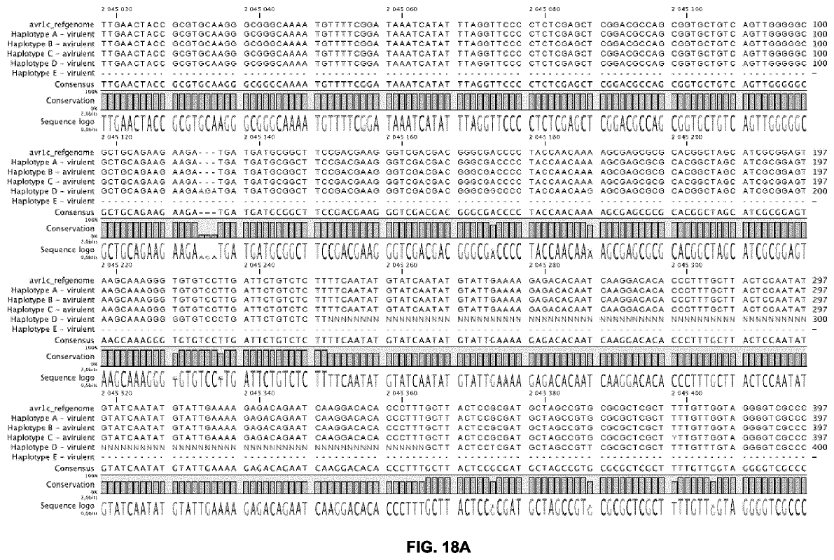

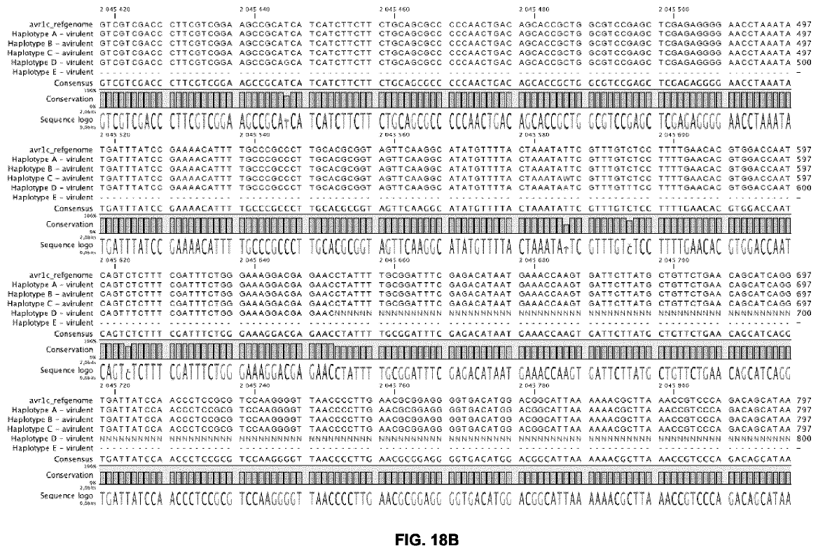

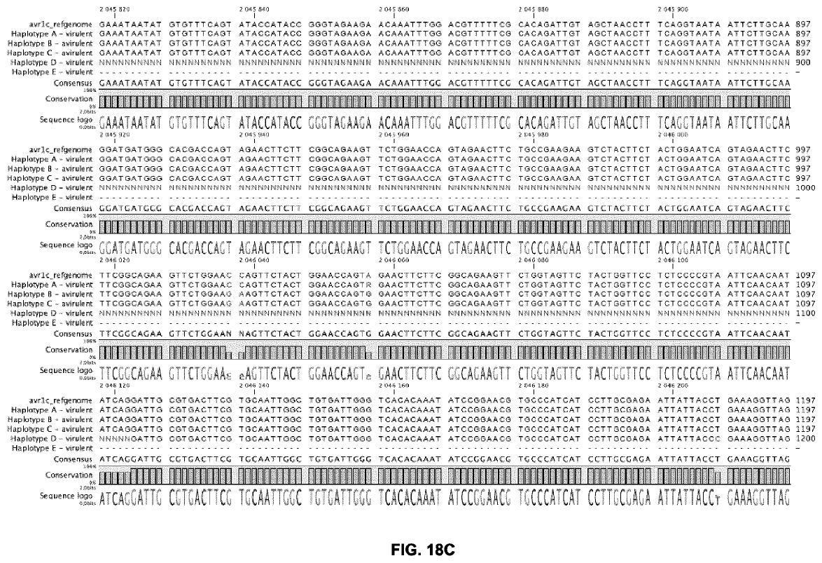

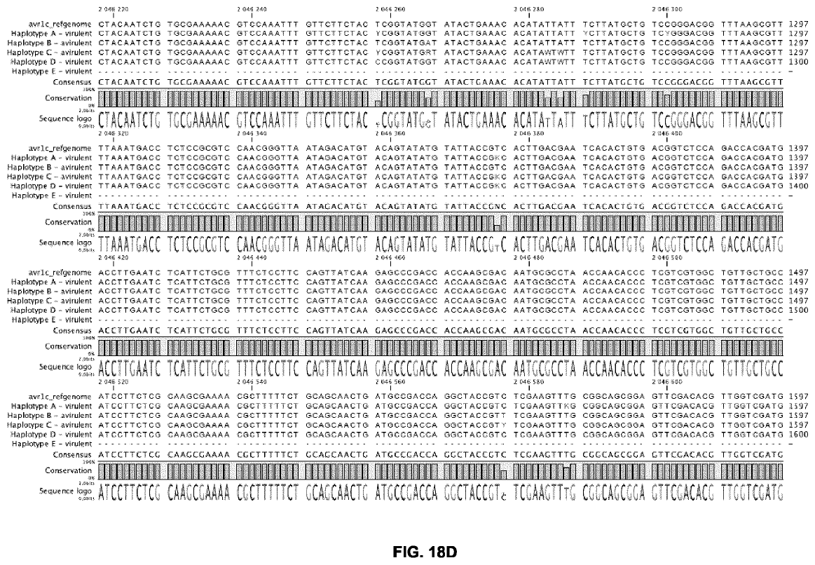

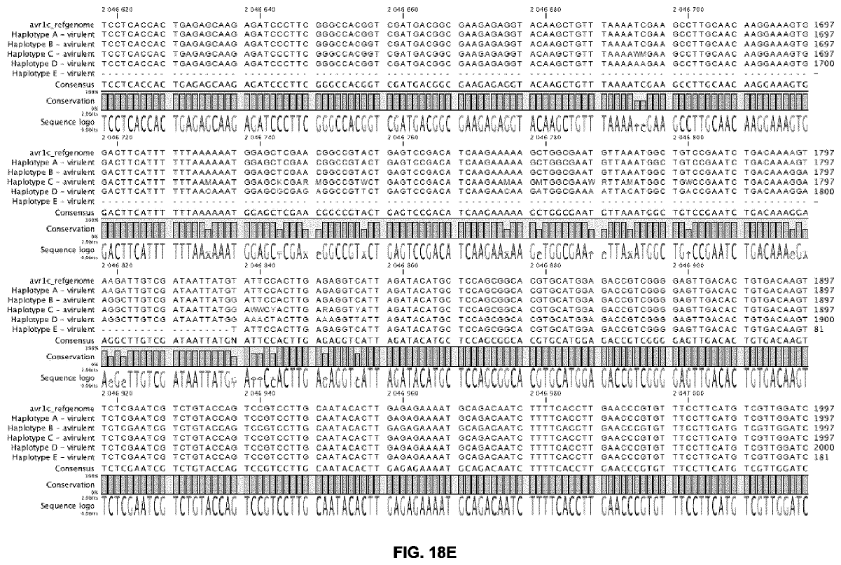

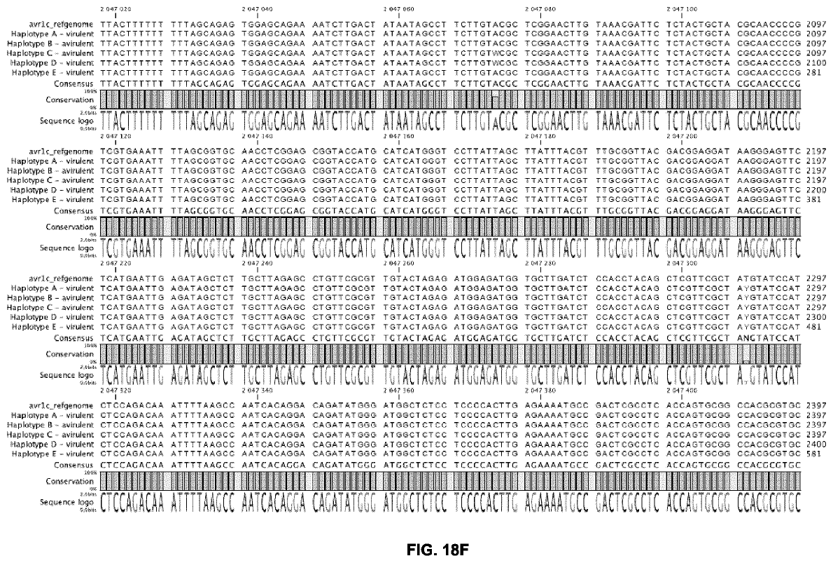

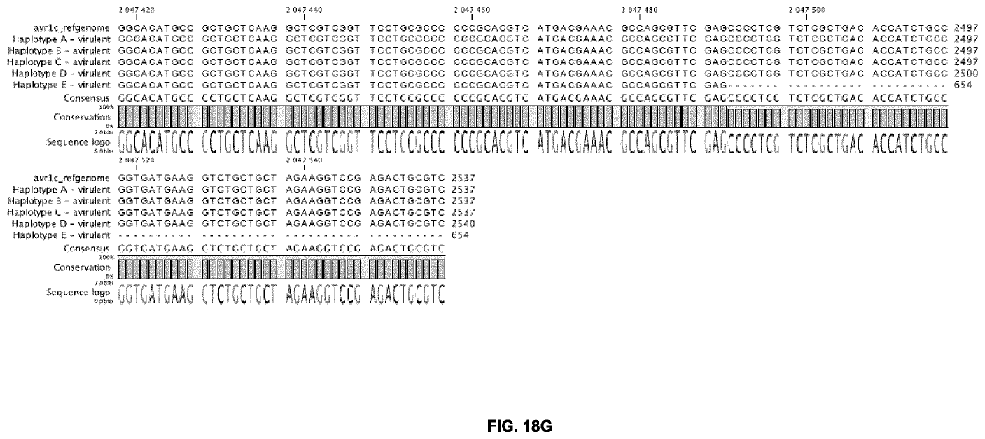

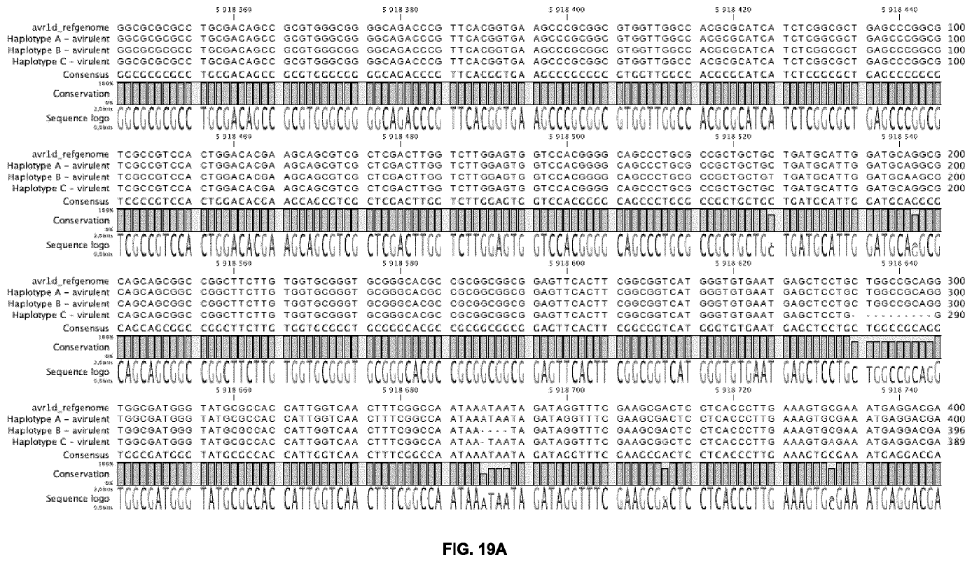

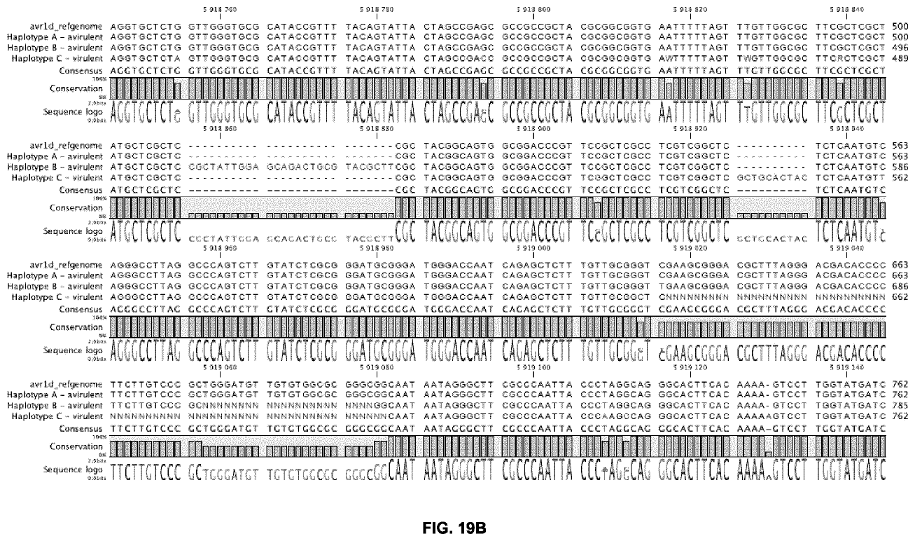

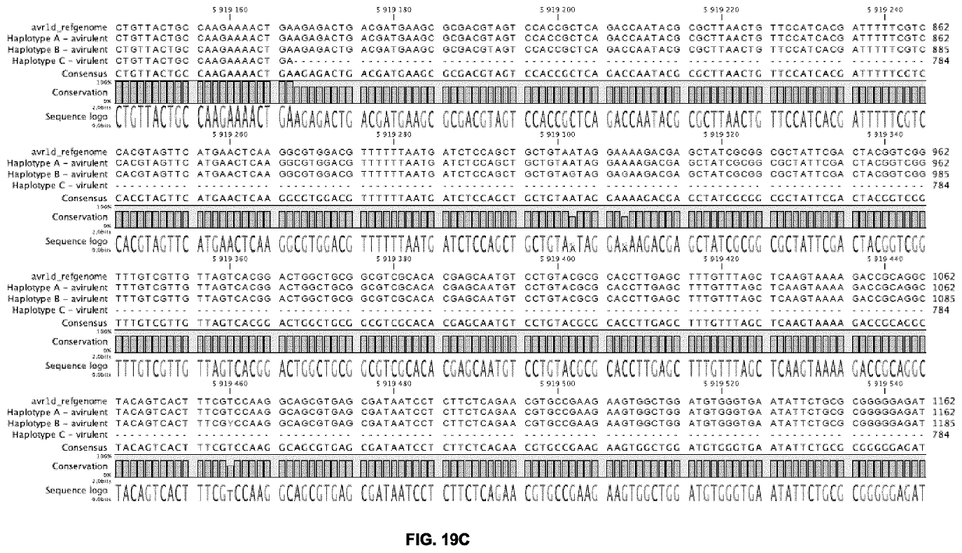

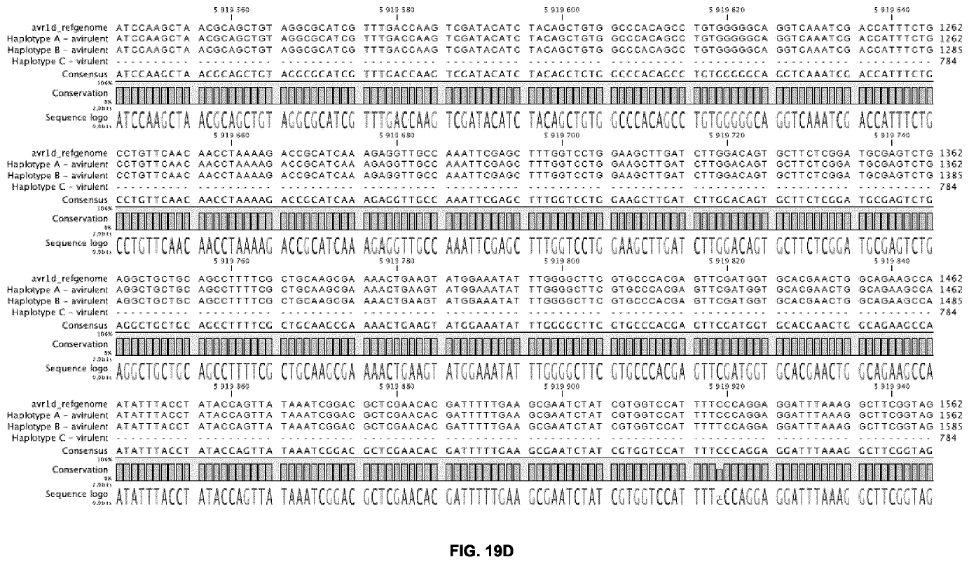

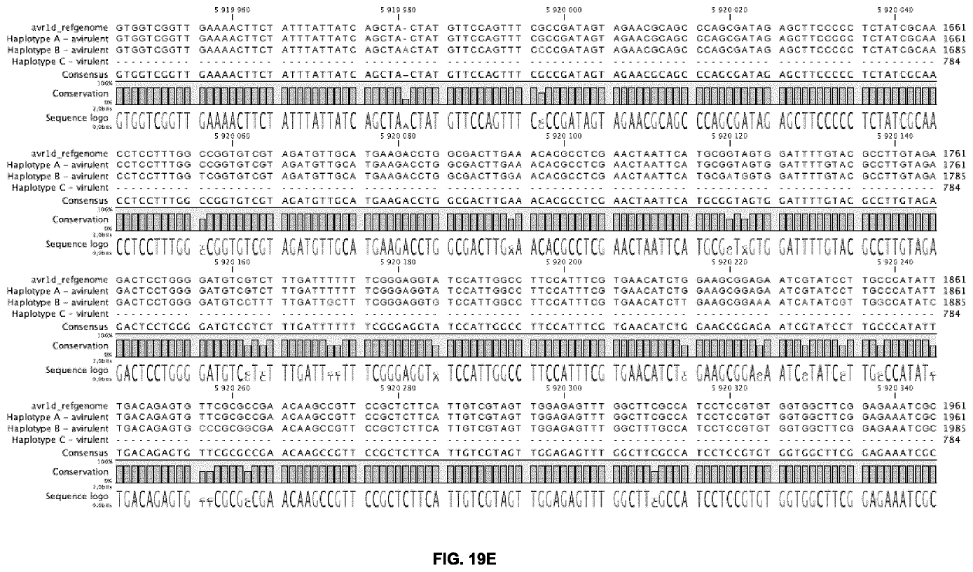

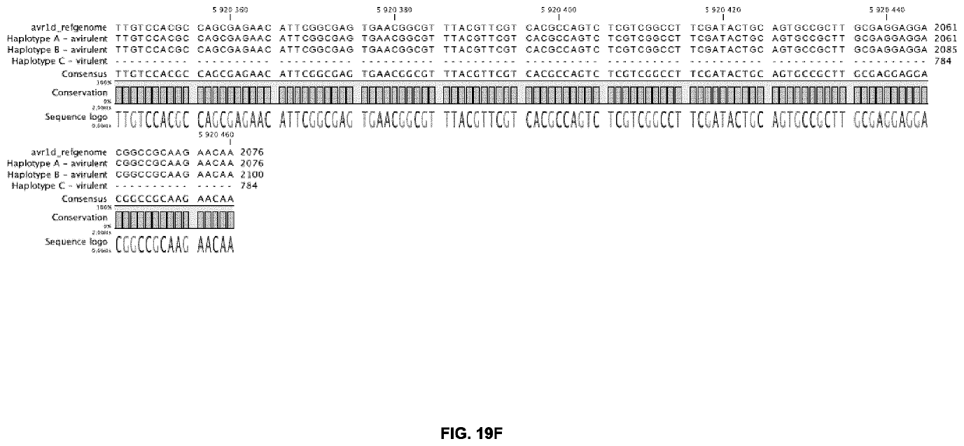

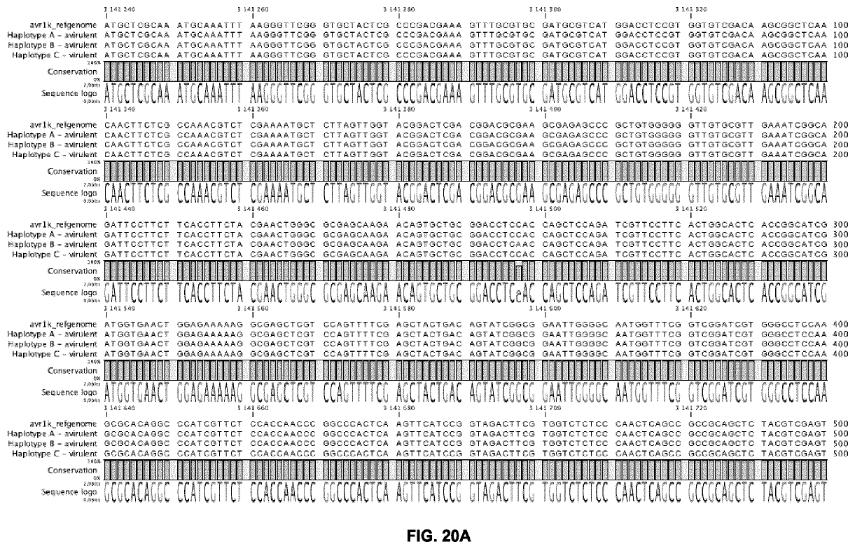

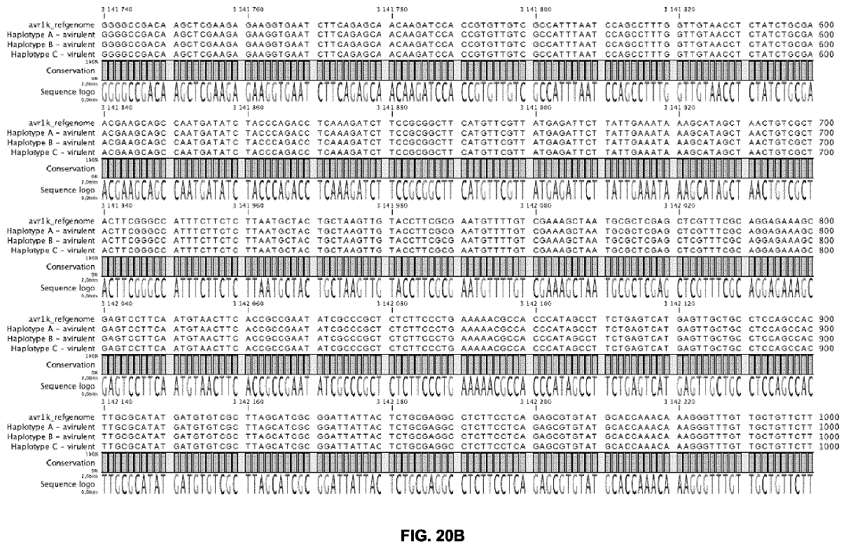

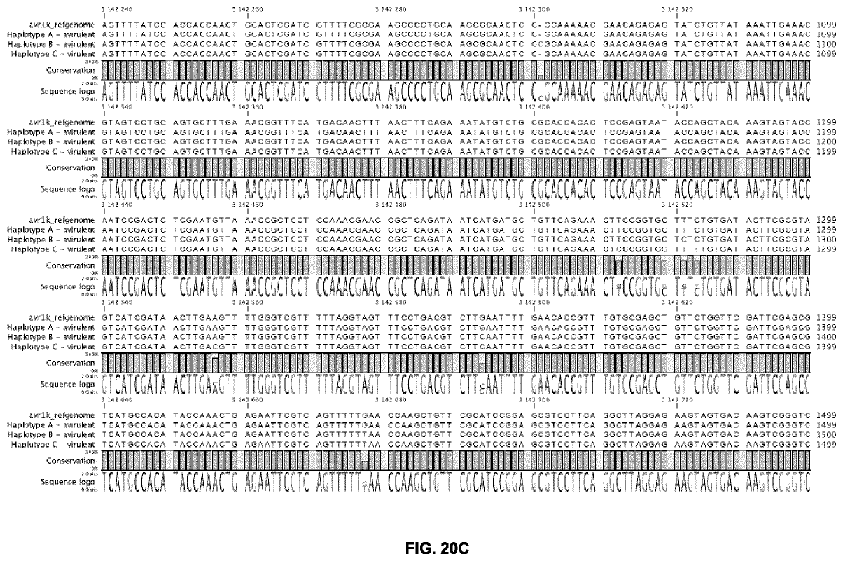

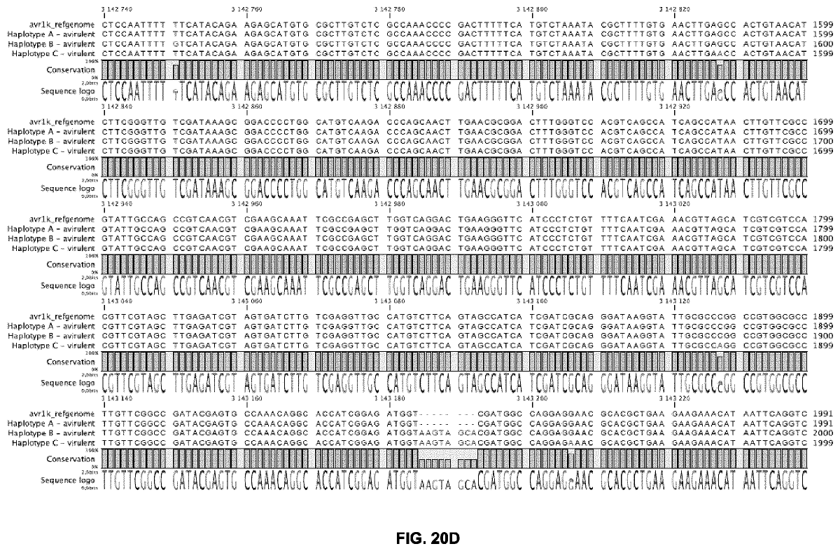

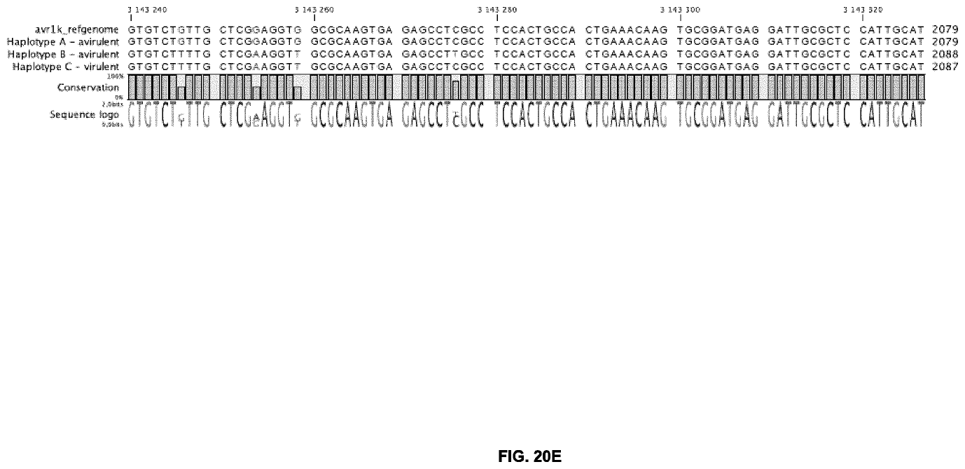

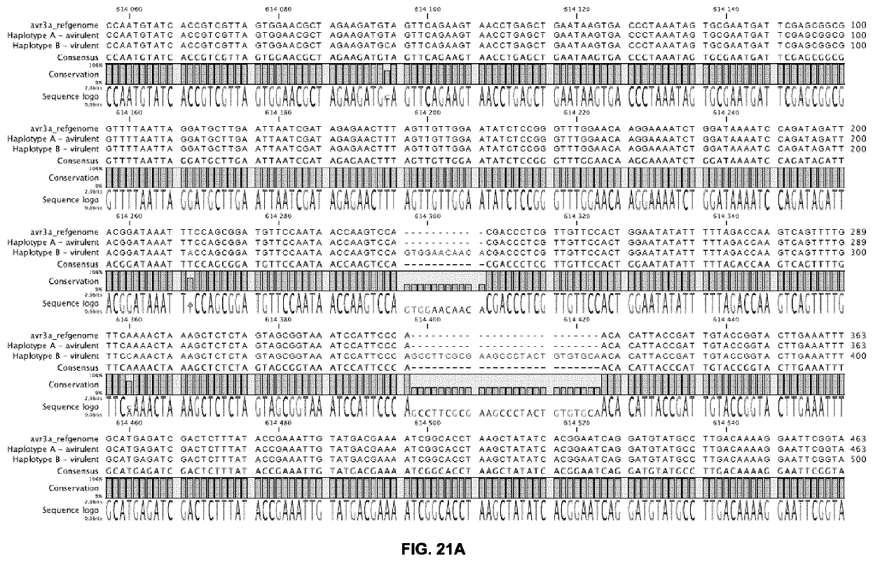

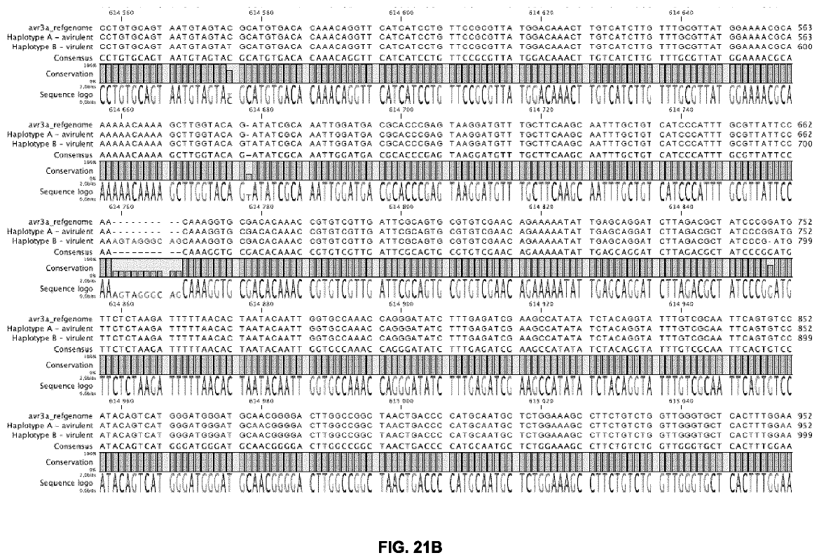

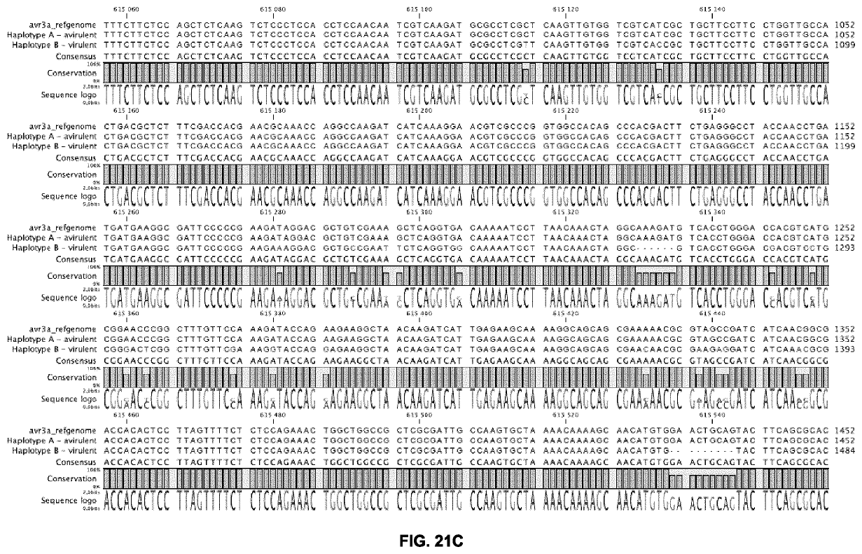

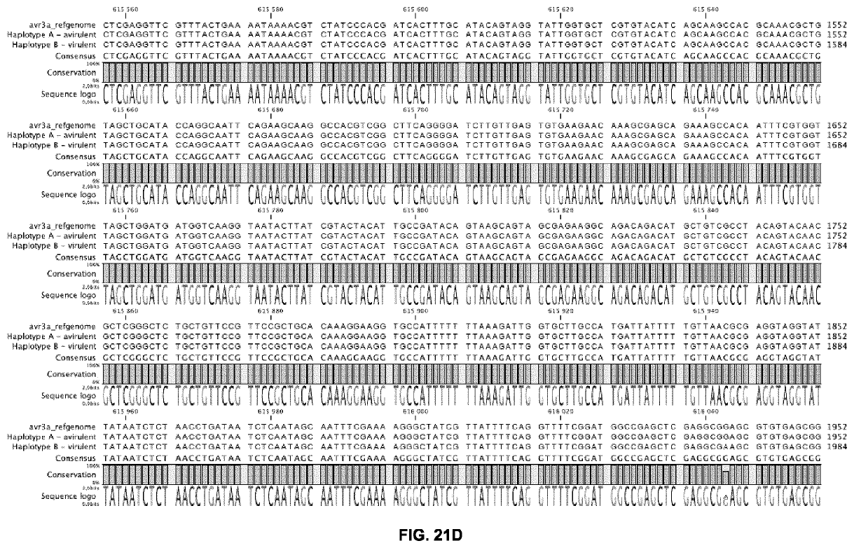

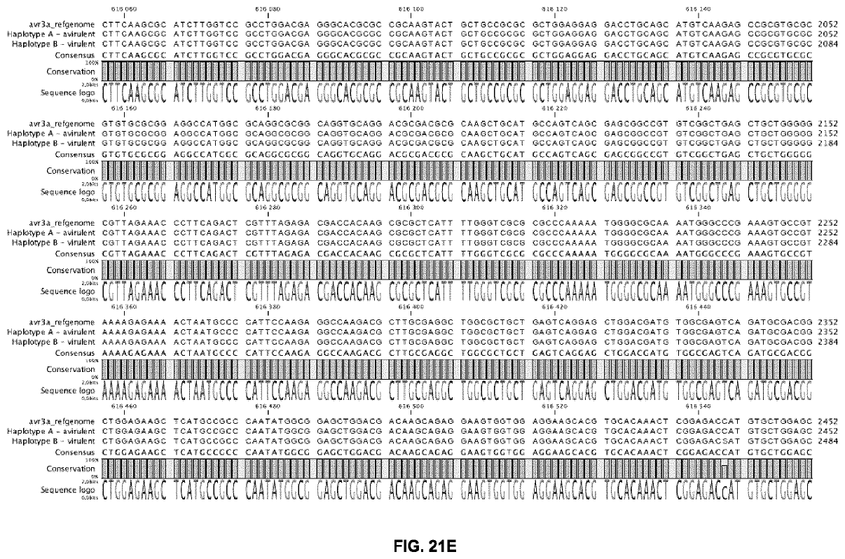

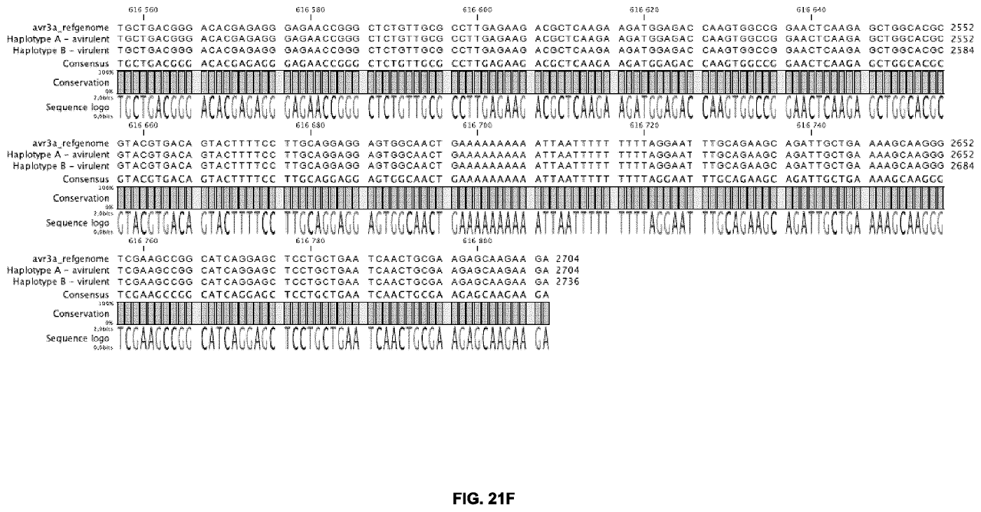

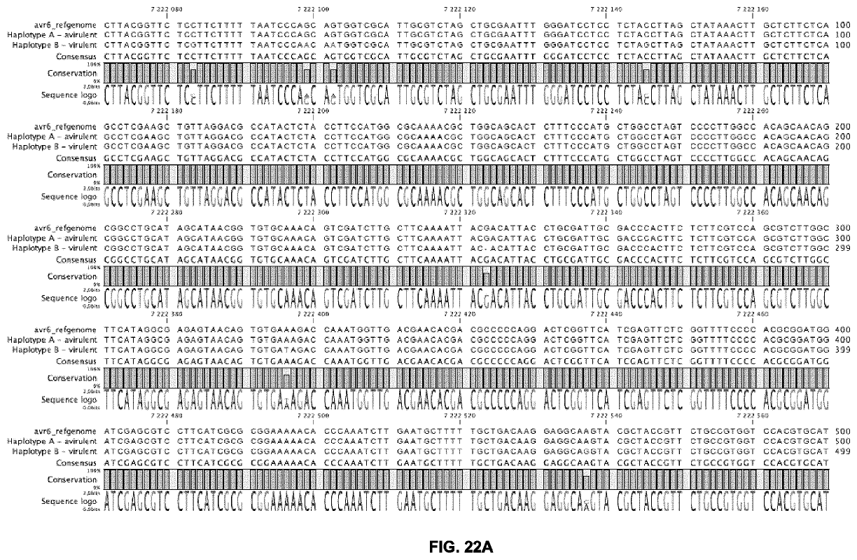

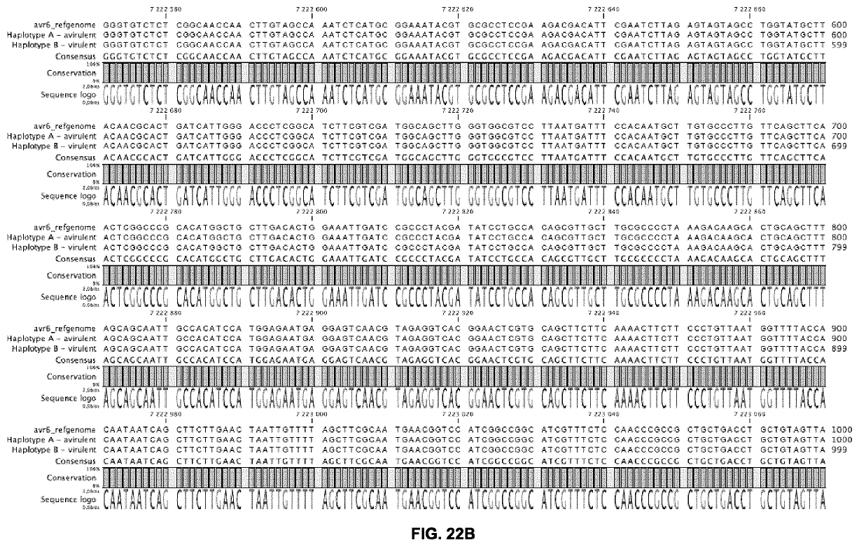

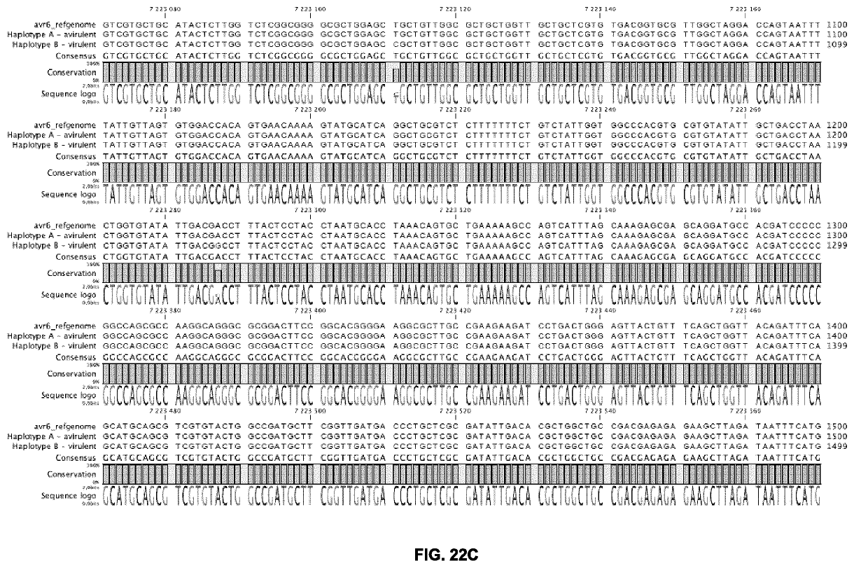

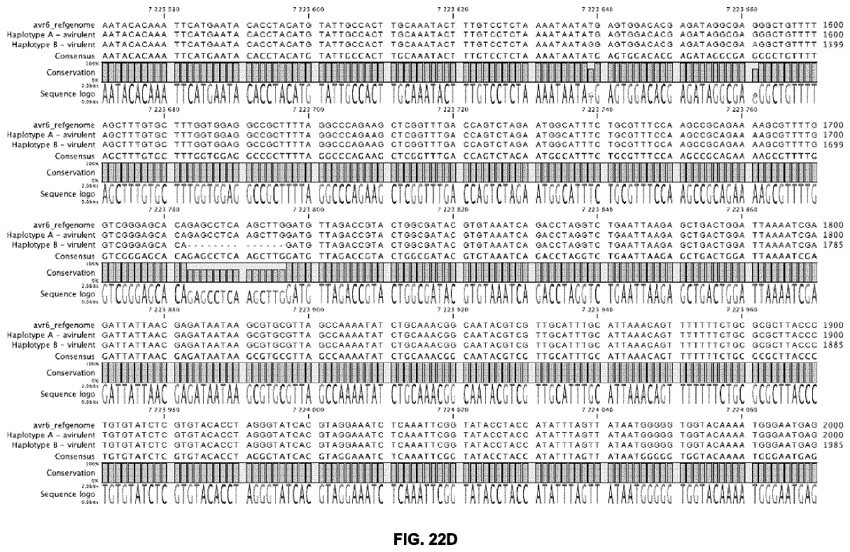

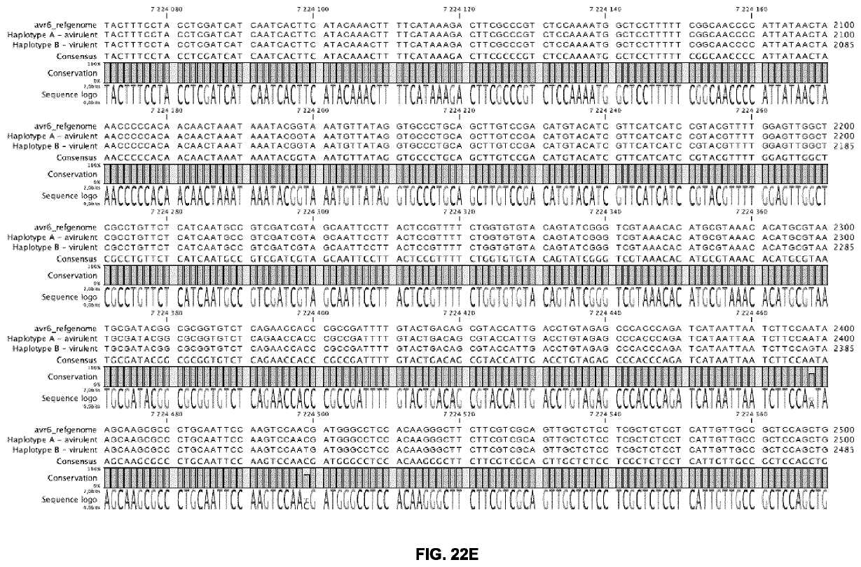

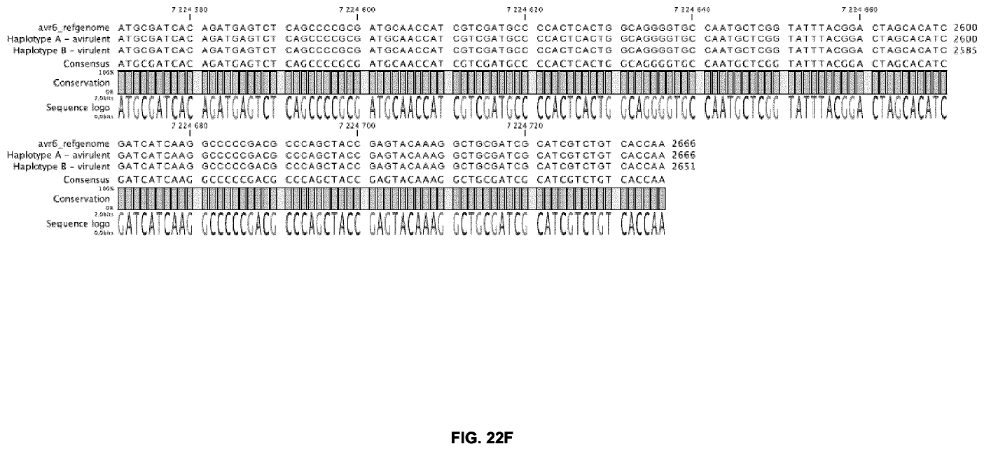

TABLE 6
Sequences described herein

| SEQ ID | |
|---|---|
| NO(s): | Description |
| 1 | avr1a_2033340_refgenome; consensus (FIG. 16A-E) |
| 2-6 | avr1a haplotypes A-E, respectively (FIG. 16A-E) |
| 7 | avr1a_refgenome (FIG. 16F-J) |
| 8-11 | avr1a haplotypes A-D, respectively (FIG. 16F-J) |
| 12 | consensus (FIG. 16F-J) |
| 13 | avr1a_2046815_refgenome (FIG. 16K-N) |
| 14-18 | avr1a haplotypes A-E, respectively (FIG. 16K-N) |
| 19 | consensus (FIG. 16K-N) |
| 20 | vr1a_2067663_refgenome; consensus (FIG. 16O-S) |
| 21-25 | avr1a haplotypes A-E, respectively (FIG. 16O-S) |
| 26 | avr1a_2214437_refgenome; consensus (FIG. 16T-X) |
| 27-31 | avr1a haplotypes A-E, respectively (FIG. 16T-X) |
| 32 | avr1a_snp1_2263686-87_refgenome (FIG. 16Y-CC) |
| 33-37 | avr1a haplotypes A-E, respectively (FIG. 16Y-CC) |
| 38 | consensus (FIG. 16Y-CC) |
| 39 | avr1a_snp2_2067663_refgenome; consensus (FIG. 16DD-HH) |
| 40-44 | avr1a haplotypes A-E, respectively (FIG. 16DD-HH) |
| 45 | avr1b_refgenome (FIG. 17A-G) |
| 46-48 | avr1b haplotypes A-C, respectively (FIG. 17A-G) |
| 49 | consensus (FIG. 17A-G) |
| 50 | avr1c_refgenome (FIG. 18A-G) |
| 51-55 | avr1c haplotypes A-E, respectively (FIG. 18A-G) |
| 56 | consensus (FIG. 18A-G) |
| 57 | avr1d_refgenome; consensus (FIG. 19A-F) |
| 58-60 | avr1d haplotypes A-C, respectively (FIG. 19A-F) |
| 61 | avr1k_refgenome (FIG. 20A-E) |
| 62-64 | avr1k haplotypes A-C, respectively (FIG. 20A-E) |
| 65 | avr3a_refgenome; consensus (FIG. 21A-F) |
| 66-67 | avr3a haplotypes A-B, respectively |
| 68 | avr6_refgenome; consensus (FIG. 22A-F) |
| 69-70 | avr6 haplotypes A-B, respectively (FIG. 22A-F) |
| 71-88 | primers (Table 2) |
| 89 | Avr6 indel (Table 4) |
| 90 | Avr6 indel (Table 4); deleted sequence |
| 91 | P. sojae genomic sequence: PHYSOscaffold_4 dna:supercontig |
| | supercontig:P_{—}sojae_V3_0:PHYSOscaffold_4:1:7609242:1 |
| 92 | P. sojae genomic sequence: PHYSOscaffold_5 dna:supercontig |
| | supercontig:P_{—}sojae_V3_0:PHYSOscaffold_5:1:6830280:1 |
| 93 | P. sojae genomic sequence: PHYSOscaffold_6 dna:supercontig |
| | supercontig:P_{—}sojae_V3_0:PHYSOscaffold_6:1:4785000:1 |
| 94 | P. sojae genomic sequence: PHYSOscaffold_7 dna:supercontig |
| | supercontig:P_{—}sojae_V3_0:PHYSOscaffold_7:1:4006126:1 |
| 95 | P. sojae genomic sequence: PHYSOscaffold_9 dna:supercontig |
| | supercontig:P_{—}sojae_V3_0:PHYSOscaffold_9:1:3285870:1 |

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Bolger, A. M., Lohse, M., and Usadel, B. 2014. Trimmomatic: a flexible trimmer for Illumina sequence data. Bioinformatics. 30:2114-2120.
Cingolani, P., Platts, A., Wang, L. L., Coon, M., Fly, T. N., 2012. A program for annotating and predicting the effects of single nucleotide polymorphisms, SnpEff: SNPs in the genome of Drosophila melanogaster strain w1118; iso-2; . . . Taylor & Francis.
DePristo, M. A., Banks, E., Poplin, R., Garimella, K. V., Maguire, J. R., Hartl, C., et al. 2011. A framework for variation discovery and genotyping using next-generation DNA sequencing data. Nature Genetics 2011 43:5. 43:491-498.
Dong, S., Yu, D., Cui, L., Qutob, D., Tedman-Jones, J., Kale, S. D., et al. 2011. Sequence Variants of the Phytophthora sojae RXLR Effector Avr3a/5 Are Differentially Recognized by Rps 3a and Rps 5 in Soybean ed. Ching-Hong Yang. PLoS ONE. 6:e20172.
Dorrance, A. E., Jia, H., and Abney, T. S. 2004. Evaluation of Soybean Differentials for Their Interaction with Phytophthora sojae. PHP.
Dou, D., Kale, S. D., Liu, T., Tang, Q., Wang, X., Arredondo, F. D., et al. 2010. Different Domains of Phytophthora sojae Effector Avr4/6 Are Recognized by Soybean Resistance Genes Rps4 and Rps6. http://dx.doi.org.acces.bibl.ulaval.ca/10.1094/MPMI-23-4-0425.23:425-435.
Gijzen, M., Forster, H., Coffey, M. D., and Tyler, B. 1996. Cosegregation of Avr4 and Avr6 in Phytophthora sojae. Canadian Journal of Botany. 74:800-802.
Goss, E. M., Press, C. M., One, N. G. P., 2013. Evolution of RXLR-class effectors in the oomycete plant pathogen Phytophthora ramorum.journals.plos.org.
Haas, J. H., and Buzzell, R. I. 1976. New races 5 and 6 of Phytophthora megasperma var. sojae and differential reactions of soybean cultivars for races 1 to 6. Phytopathology.
Kadam, S., Vuong, T. D., Qiu, D., Meinhardt, C. G., Song, L., Deshmukh, R., et al. 2016. Genomic-assisted phylogenetic analysis and marker development for next generation soybean cyst nematode resistance breeding. Plant Science. 242:342-350.
Kamoun, S., Furzer, O., Jones, J. D. G., Judelson, H. S., Ali, G. S., Dalio, R. J. D., et al. 2015. The Top 10 oomycete pathogens in molecular plant pathology. Molecular Plant Pathology. 16:413-434.
Kilen, T. C., Hartwig, E. E., and Keeling, B. L. 1974. Inheritance of a Second Major Gene for Resistance to Phytophthora Rot in Soybeans 1. Crop Science. 14:260-262.
Knaus, B. J., and Grünwald, N. J. 2017. vcfr: a package to manipulate and visualize variant call format data in R. Molecular Ecology Resources. 17:44-53.
Lebreton, A., Labbe, M. C., de Ronne, M. M., Xue, D. A., Marchand, M. G., and Belanger, D. R. R. 2018. Development of a simple hydroponic assay to study vertical and horizontal resistance of soybean and pathotypes of Phytophthora sojae. https://doi-org.acces.bibl.ulaval.ca/10.1094/PDIS-04-17-0586-RE. :PDIS-04-17-0586-RE.
Li, H., and Durbin, R. 2009. Fast and accurate short read alignment with Burrows-Wheeler transform. Bioinformatics. 25:1754-1760.
MacGregor, T., Bhattacharyya, M., Tyler, B., Bhat, R., Schmitthenner, A. F., and Gijzen, M. 2002. Genetic and Physical Mapping of Avr1a in Phytophthora sojae. Genetics. 160:949-959.
May, K. J., Whisson, S. C., Zwart, R. S., Searle, I. R., Irwin, J. A. G., MacLean, D. J., et al. 2002. Inheritance and mapping of 11 avirulence genes in Phytophthora sojae. Fungal Genetics and Biology. 37:1-12.
Morrison, R. H., and Thorne, J. C. 1978. Inoculation of Detached Cotyledons For Screening Soybeans Against Two Races of Phytophthora Megasperma Var. Sojae 1. Crop Science. 18:1089-1091.
Na, R., Yu, D., Chapman, B. P., Zhang, Y., Kuflu, K., Austin, R., et al. 2014. Genome Re-Sequencing and Functional Analysis Places the Phytophthora sojae Avirulence Genes Avr1c and Avr1a in a Tandem Repeat at a Single Locus ed.
Niklaus J Grünwald. PLoS ONE. 9:e89738.
Pazdernik, D. L., Hartman, G. L., Huang, Y. H., and Hymowitz, T. 2007. A Greenhouse Technique for Assessing Phytophthora Root Rot Resistance in Glycine max and G. soja.
http://dx.doi.org.acces.bibl.ulaval.ca/10.1094/PDIS.1997.81.10.1112.81:1112-1114.
Pfaffl, M. W., Horgan, G. W., research, L. D. N. A., 2002. Relative expression software tool (REST©) for group-wise comparison and statistical analysis of relative expression results in real-time PCR. academic.oup.com.
Qutob, D., Tedman-Jones, J., Dong, S., Kuflu, K., Pham, H., Wang, Y., et al. 2009. Copy Number Variation and Transcriptional Polymorphisms of Phytophthora sojae RXLR Effector Genes Avr1a and Avr3a ed. Frederick M Ausubel. PLoS ONE. 4:e5066.
Raffaele, S., Farrer, R. A., Cano, L. M., Studholme, D. J., MacLean, D., Thines, M., et al. 2010. Genome Evolution Following Host Jumps in the Irish Potato Famine Pathogen Lineage. Science. 330:1540-1543.
Ruijter, J. M., Ramakers, C., acids, W. H. N., 2009. Amplification efficiency: linking baseline and bias in the analysis of quantitative PCR data. academic.oup.com.
Sahoo, D. K., Abeysekara, N. S., Cianzio, S. R., one, A. R. P., 2017. A Novel Phytophthora sojae Resistance Rps12 Gene Mapped to a Genomic Region That Contains Several Rps Genes. journals.plos.org.
Schmitthenner, A. F., Hobe, M., and Bhat, R. G. 1994. Phytophthora sojae races in Ohio over a 10-year interval. Plant Disease. 78:269-276.
Song, T., Kale, S. D., Arredondo, F. D., Shen, D., Su, L., Liu, L., et al. 2013. Two RxLR Avirulence Genes in Phytophthora sojae Determine Soybean Rps1k-Mediated Disease Resistance.
http://dx.doi.org.acces.bibl.ulaval.ca/10.1094/MPMI-12-12-0289-R. 26:711-720.
Tardivel, A., Sonah, H., Belzile, F., and O'Donoughue, L. S. 2014. Rapid Identification of Alleles at the Soybean Maturity Gene E3 using genotyping by Sequencing and a Haplotype-Based Approach. The Plant Genome. 7:0.
Tyler, B. M., and Gijzen, M. 2014. The Phytophthora sojae Genome Sequence: Foundation for a Revolution. In Genomics of Plant-Associated Fungi and Oomycetes: Dicot Pathogens, Berlin, Heidelberg: Springer Berlin Heidelberg, p. 133-157.
Tyler, B. M., Forster, H., Plant-Microbe, M. C. M., 1995. Inheritance of avirulence factors and restriction fragment length polymorphism markers in outcrosses of the oomycete Phytophthora sojae. Molecular Plant-Microbe Interactions.
Tyler, B. M., Tripathy, S., Zhang, X., Dehal, P., Jiang, R. H. Y., Aerts, A., et al. 2006. Phytophthora Genome Sequences Uncover Evolutionary Origins and Mechanisms of Pathogenesis. Science. 313:1261-1266.
Verta, J. P., Landry, C. R., and MacKay, J. 2016. Dissection of expression-quantitative trait locus and allele specificity using a haploid/diploid plant system—insights into compensatory evolution of transcriptional regulation within populations. New Phytologist. 211:159-171.
Wagner, R. E., Wilkinson, H. W. P., 1992. An aeroponics system for investigating disease development on soybean taproots infected with Phytophthora sojae. apsnet.org.
Ward, E., Lazarovits, G., Unwin, C. H., Phytopathology, R. B., 1979. Hypocotyl reactions and glyceollin in soybeans inoculated with zoospores of Phytophthora megasperma var. sojae. apsnet.org.
Whisson, S. C. 1995. Phytophthora sojaeAvirulence Genes, RAPD, and RFLP Markers Used to Construct a Detailed Genetic Linkage Map. Molecular Plant-Microbe Interactions. 8:988.
Xue, A. G., Marchand, G., Chen, Y., Zhang, S., Cober, E. R., and Tenuta, A. 2015. Races of Phytophthora sojae in Ontario, Canada, 2010-2012. Canadian Journal of Plant Pathology. 37:376-383.
Zeng, P., Zhou, X., and Huang, S. 2017. Prediction of gene expression with cis-SNPs using mixed models and regularization methods. BMC Genomics 2017 18:1. 18:368

1. A method for assessing whether a Phytophthora pathogen is virulent or avirulent, comprising:
(a) determining, in a sample comprising Phytophthora nucleic acid, the presence or absence of one or more Avr gene variations in the Phytophthora nucleic acid; and
(b) determining whether the Phytophthora pathogen is virulent or avirulent on the basis of the presence or absence of the one or more variations.

2. The method of claim 1, wherein the one or more variations are comprised in one or more of Avr1a, Avr1b, Avr1c, Avr1d, Avr1k, Avr3a and Avr6 or a flanking region thereof.

3. The method of claim 1 or 2, wherein the one or more variations are each independently a substitution, deletion or insertion of one or more nucleotides.

4. (canceled)

5. The method of claim 1, wherein the variations are one or more indels and/or SNPs corresponding to one or more indels and/or SNPs set forth in FIGS. 1 (Avr1a), 3 (Avr1b), 5 (Avr1c), 7 (Avr1c), 9 (Avr1k), 11 (Avr3a) and/or 12 (Avr6), 16-23 and/or Table 4 and/or 5.

6. (canceled)

7. The method of claim 1, wherein the presence or absence of the one or more variations is determined using an amplification method.

8-9. (canceled)

10. The method of claim 7, wherein the amplification is carried out as one or more multiplex amplifications for determination of the presence or absence of two or more variations in each amplification reaction.

11. The method of claim 10, wherein the one or more multiplex amplifications comprises or consists of two multiplex amplifications.

12. The method of claim 11, wherein the two multiplex amplifications comprise (a) a first multiplex amplification to determine the presence or absence of one or more indels and/or SNPs corresponding to one or more indels and/or SNPs in Avr1a shown in FIGS. 1, 16 and/or 23, and/or Table 4 and/or 5 one or more indels and/or SNPs corresponding to one or more indels and/or SNPs in Avr1b, Avr1d, Avr1k, Avr3a and Avr6 shown in FIGS. 3, 7, 9, 11, 13 and/or 17 and 19-23, and/or Table 4 and/or 5; and (b) a second amplification to determine the presence or absence of one or more indels and/or SNPs corresponding to one or more indels and/or SNPs in Avr1c shown in FIGS. 5, 18, and/or 23, and/or Table 4 and/or 5.

13-16. (canceled)

17. The method of claim 1, wherein the Phytophthora pathogen is Phytophthora sojae.

18. A method for assessing risk of Phytophthora pathogen infection of a soybean plant:
(a) assessing, in a sample obtained from the plant, the soil, the water, the seeds, the air, or any culture containing one or several isolates of Phytophthora pathogen, whether the sample comprises a virulent or avirulent Phytophthora pathogen using the method of claim 1; and
(b) assessing the risk of Phytophthora pathogen infection of the soybean plant on the basis of the assessment made in (a), wherein the presence of a virulent Phytophthora pathogen in the sample is indicative of an elevated risk of Phytophthora pathogen infection of the soybean plant.

19. (canceled)

20. A method for selecting a soybean cultivar for planting in an agricultural area, comprising:
(a) assessing, in a sample obtained from the plant, the soil, the water, the seeds, the air, or any culture containing one or several isolates of Phytophthora pathogen, whether the sample comprises a virulent or avirulent Phytophthora pathogen using the method of claim 1;
(b) if the sample comprises a virulent Phytophthora pathogen, selecting a soybean cultivar comprising one or more resistances (Rps) genes that confer resistance to the one or more Avr genes identified in the sample that confer virulence, for planting in the agricultural area.

21. A collection, kit or package comprising one or more oligonucleotides for determining the presence or absence of one or more Avr gene variations in the nucleic acid of a Phytophthora pathogen.

22. The collection, kit or package of claim 21, wherein the one or more variations are comprised in one or more of Avr1a, Avr1b, Avr1c, Avr1d, Avr1k, Avr3a and Avr6 or a flanking region thereof.

23. The collection, kit or package of claim 21 or 22, wherein the one or more variations are each independently a substitution, deletion or insertion of one or more nucleotides.

24. (canceled)

25. The collection, kit or package of claim 21, wherein the variations are one or more indels and/or SNPs corresponding to one or more indels and/or SNPs set forth in FIGS. 1 (Avr1a), 3 (Avr1b), 5 (Avr1c), 7 (Avr1d), 9 (Avr1k), 11 (Avr3a) and/or 12 (Avr6), 16-23 and/or Table 4 and/or 5.

26. (canceled)

27. The collection, kit or package of claim 21, wherein the presence or absence of the one or more variations is determined using an amplification method.

28-29. (canceled)

30. The collection, kit or package of claim 27, wherein the one or more oligonucleotides are for use in one or more multiplex amplifications for determination of the presence or absence of two or more variations in each amplification reaction.

31. The collection, kit or package of claim 30, wherein the one or more multiplex amplifications comprises or consists of two multiplex amplifications.

32. The collection, kit or package of claim 31, wherein the two multiplex amplifications comprise (a) a first multiplex amplification to determine the presence or absence of one or more indels and/or SNPs corresponding to one or more indels and/or SNPs in Avr1a shown in FIGS. 1, 16 and/or 23, and/or Table 4 and/or 5 one or more indels and/or SNPs corresponding to one or more indels and/or SNPs in Avr1b, Avr1d, Avr1k, Avr3a and Avr6 shown in FIGS. 3, 7, 9, 11, 13 and/or 17 and 19-23, and/or Table 4 and/or 5; and (b) a second amplification to determine the presence or absence of one or more indels and/or SNPs corresponding to one or more indels and/or SNPs in Avr1c shown in FIGS. 5, 18, and/or 23, and/or Table 4 and/or 5.

33-36. (canceled)

37. The collection, kit or package of claim 21, wherein the Phytophthora pathogen is Phytophthora sojae.

38. (canceled)
